(19) United States
(12) Patent Application Publication
(10) Pub. No.: US 2022/0372147 A1
(43) Pub. Date: Nov. 24, 2022

(54) BINDING MODULES COMPRISING MODIFIED EHD2 DOMAINS

(71) Applicant: UNIVERSITÄT STUTTGART, Stuttgart (DE)

(72) Inventors: Roland KONTERMANN, Nürtingen (DE); Oliver SEIFERT, Stuttgart (DE)

(21) Appl. No.: 17/762,463

(22) PCT Filed: Sep. 25, 2020
(86) PCT No.: PCT/EP2020/077008

§ 371 Date: Mar. 22, 2022

(30) Foreign Application Priority Data
Sep. 25, 2019 (EP) 19199639.6

(51) Int. Cl. C07K 16/28; C07K 16/46

(52) U.S. Cl. CPC C07K 16/2809; C07K 16/468; C07K 2317/35; C07K 2317/52

(57) ABSTRACT

The present invention relates to binding molecules comprising two polypeptide chains, wherein the peptide chains comprise modified EHD2 domains allowing heterodimerization only, thereby preventing homodimers, nucleic acids encoding such binding molecules and uses of such binding molecules or nucleic acids encoding such binding molecules in therapy.

The present invention relates to binding molecules comprising two polypeptide chains, wherein the peptide chains comprise modified EHD2 domains allowing heterodimerization only, thereby preventing homodimers. The present invention further pertains to nucleic acids encoding such binding molecules and uses of such binding molecules or nucleic acids encoding such binding molecules in therapy.

### BACKGROUND OF THE INVENTION

Antibodies with at least two different specificities—so-called bispecific antibodies—have attracted increasing interest for a broad spectrum of applications that include diagnosis, imaging, prophylaxis and therapy. In addition to retargeting of effector molecules, cells and genetic vehicles, dual targeting and pretargeting strategies, mimicry of the natural function of proteins, half-life extension, and delivery through biological barriers such as the blood-brain barrier have been utilized (Labrijn et al., 2019, Nat. Rev. Drug. Discov. 18: 585-608). Bispecific antibodies have been evaluated as potential treatments for a variety of indications, including cancer, chronic inflammatory diseases, autoimmunity, neurodegeneration, bleeding disorders, and infections.

Bispecific antibodies can be classified according to format and composition (Brinkmann & Kontermann, 2017, MAbs 9: 182-212). A main discrimination is the presence or absence of an Fc region. Bispecific antibodies with no Fc will lack Fc-mediated effector functions, such as antibody-dependent cell-mediated cytotoxicity (ADCC), antibody-dependent cellular phagocytosis (ADCP), complement fixation, and FcRn-mediated recycling, which is responsible for the long half-life of most γ immunoglobulins. Bispecific antibodies that include an Fc region can be further divided into those that exhibit a structure resembling that of an IgG molecule and those that contain additional binding sites, i.e., those with an appended or modified Ig-like structure.

The generation of bivalent, bispecific IgGs or Ig-like molecules is, however, complicated by the fact that the antigen-binding sites are built by the variable domains of the light and heavy chain (VL, VH). Promiscuous pairing of heavy and light chains of two antibodies expressed in one cell can theoretically result in 16 different combinations (10 different molecules), with only one being bispecific and the remaining pairings resulting in non-functional or monospecific molecules. To direct and force correct assembly of correct binding sites, i.e., heavy and light chains of the correct specificity, is one of the challenges of generating bispecific antibodies. Various strategies have been developed and established over the past two decades to address these problems.

Fusion of two antibody-producing cell lines, e.g., generating a hybrid-hybridoma (quadroma), allows the combination of the heavy and light chains of two different antibodies. The resulting bispecific antibodies thus comprise the heavy and light chain of the first antibody and the heavy and light chain of the second antibody. Heavy and light chain constant regions can be of the same isotype, but can also be of different isotypes. They can even be from different species, a strategy utilized to generate triomabs. In this format, a mouse hybridoma is fused with a rat hybridoma, resulting in production of a bispecific, asymmetric hybrid IgG molecule. Preferential pairing of light chains with its corresponding heavy chain was described. Importantly, the heteromeric Fc part allows fractionated purification by protein A chromatography because of reduced binding, and elution from the column occurs already at a pH of around 5.8. Furthermore, cell lines producing two different heavy and light chains can be generated by genetic means. This allows use of heavy and light chains of defined composition, e.g., certain human isotypes, and implementation of mutated sequences, still random pairing of the heavy and light chains represents one of the major obstacles of these approaches.

Genetic engineering to force heterodimerization of heavy chains solves one of the problems of bispecific IgG formation, e.g. knobs-into-holes mutations and electrostatic steering mutations introduced into the C_{H}3 domains (Krah et al., 2017, N. Biotechnol. 39: 167-173). However, heterodimeric heavy chains can still assemble with two different light chains, resulting in four possible combinations, one bispecific molecule, one non-functional combination, and two monospecific molecules. Approaches have, therefore, been developed to allow the correct pairing of cognate heavy and light chains in combination with Fc-modified heavy chains. One approach is the use of a common light chain used in both heavy chains of an IgG molecule to form a functional binding site (Merchant et al., 1998, Nat. Biotechnol. 16: 677-681). However, this requires the identification and isolation of antigen-binding sites utilizing the same VL domain.

Alternatively, mutations and modifications have been introduced into the light chain and Fd fragment (V_{H}-C_{H}1 fragment) of the heavy chain of one of the binding sites to force correct pairing of heavy and light chains (Krah et al., 2017, N. Biotechnol. 39: 167-173). These strategies include modifications of the C_{H}1 and C_{L} domains of one heavy and light chain, and in some cases also of the V_{H} and V_{L} domains. Examples are the CrossMab technology where the C_{H}1 and C_{L} domains are exchanged between one heavy and light chain (Klein et al., 2012, MAbs 4: 653-663), orthogonal Fab mutations introduced into the C_{H}1 and C_{L} domains (Lewis et al., 2014, Nat. Biotechnol. 32: 191-198), or in addition into the V_{H} and V_{L} domains (Golay et al., 2016, J. Immunol. 196: 199-211; Froning et al., 2017, Protein Sci. 26: 2021-2038; Bönisch et al., 2017, Protein Eng. Des. Sel., 2017, 30: 685-696), and the Duetmab technology where the natural disulfide bond between C_{H}1 and C_{L} is removed and substituted by a new, artificially introduced disulfide bond (Mazor et al., 2015, MAbs 7: 461-469).

Another approach is the substitution of the C_{H}1 and C_{L} domain of one heavy and light chain by structurally related domains from other proteins, such as the C-alpha and C-beta domains from the T-cell receptor (TCR) (Wu et al., 2015, MAbs 7: 470-482). Alternatively, the C_{H}1 and C_{L} domains were substituted by a mutated heavy chain domain 2 from IgE, which naturally forms homodimers. The mutations introduced into this so-called EFab module allowed to form a knob-into-hole-like structure reducing homodimerization and allowing formation of heterodimeric Fab-like molecules (Cooke et al., 2018, MAbs 10: 1248-1259). However, a general problem of these knob-into-hole mutations is the likelihood of formation of homodimeric interactions of the hole mutation-containing domains. In the EFab format, these hole mutations are present in the light chain, thus bearing the risk of formation of light chain homodimers (Kuglstatter et al., 2017, Protein Eng. Des. Sel. 30: 649-656). Furthermore, a reduced thermal stability and an increased susceptibility for proteolytic cleavage was observed for EFab compared to wild-type Fab fragments and the wild-type EHD2 Fab molecule, eventually due to disordered regions in the interface between these mutant domains (Cooke et al., 2018, MAbs 10: 1248-1259).

Further mutations to generate heterodimerizing EHD2 domains have been proposed recently (WO 2017/011342 A1 ). Here, several mutations were described that can be introduced into either the EHD2 (EH2) domain or, alternatively, the structurally and functionally related MHD2 (MH2) domain (Seifert et al., 2014, Mol. Cancer Ther. 13: 101-111), to allow heterodimerization through electrostatic or hydrophobic interactions. All examples reduced to practice, however, used solely the MH2 derivatives. Nevertheless, in these derivatives a substantial number of residues had to be modified from the wild-type sequence.

There remains a need in the art for dimerizing domains that exclude homodimerization of polypeptides in binding molecules to a satisfying degree and, thus provides a solution of the light and heavy chain pairing problem, i.e. the mispairing of light and heavy chains in a multi specific, preferably bispecific antibody.

### SUMMARY OF THE INVENTION

The present inventors discovered that a first polypeptide chain comprising a first modified EHD2 domain co-expressed with a second polypeptide chain comprising a second modified EHD2 domain substantially prevents formation of homodimers and at the same time allows formation of heterodimers to a satisfying degree, rendering the modified domains suitable for inter alia large scale production of binding molecules.

Thus, according to a first aspect, the present invention provides a binding molecule comprising a first polypeptide chain comprising a first binding domain (BD1) and a first modified EHD2 domain (EHD2-1), and a second polypeptide chain comprising a second binding domain (BD2) and a second modified EHD2 domain (EHD2-2), wherein the amino acid sequences of EHD2-1 and EHD2-2 are different from each other and each is selected from an amino acid sequence with at least 70% amino acid identity to SEQ ID NO:1 and which does not have a Cys at position 14, or an amino acid sequence with at least 70% amino acid identity to SEQ ID NO:1 and which does not have a Cys at position 102, wherein BD1 and BD2 together form an antigen binding site, and wherein EHD2-1 and EHD2-2 are covalently bound to each other.

According to one embodiment, one or both of the modified EHD2 domains further comprises a single amino acid substitution at position N39.

According to a further embodiment, BD1 and BD2 are different from each other and each is selected from a variable heavy chain (V_{H}) and a variable light chain (V_{L}) or from a variable region of a TCR α-chain and a variable region of a TCR β-chain.

According to yet another embodiment, the binding molecule further comprises a first Fc chain.

According to a further embodiment, the binding molecule further comprises a third binding domain (BD3) and a fourth binding domain (BD4), wherein BD3 and BD4 together form an antigen binding site.

According to a further embodiment, BD3 and BD4 are different from each other and each is selected from a V_{H} and a V_{L} or from a variable region of a TCR α-chain and a variable region of a TCR β-chain.

According to a further embodiment, the binding molecule further comprises a third polypeptide chain. According to a preferred embodiment, the binding molecule further comprises a third and a fourth polypeptide chain.

According to yet another embodiment, the binding molecule further comprises a second Fc chain.

According to one embodiment, the first and the second Fc chains are different from each other and form a heterodimeric Fc.

According to a further embodiment, the binding molecule is monospecific or bispecific.

According to yet another embodiment, the third polypeptide chain comprising BD3 further comprises one of

(i) a C_{H}1 domain,

(ii) a C_{L} domain,

(iii) a first modified EHD2 domain (EHD2-1), and

(iv) a second modified EHD2 domain (EHD2-2),

and wherein the fourth polypeptide chain comprising BD4 further comprises

in case of (i) a C_{L} domain,

in case of (ii) a C_{H}1 domain,

in case of (iii) a second modified EHD2 domain (EHD2-2), and

in case of (iv) a first modified EHD2 domain (EHD2-1),

wherein the amino acid sequences of EHD2-1 and EHD2-2 are different from each other and each is selected from an amino acid sequence with at least 70% amino acid identity to SEQ ID NO:1 and which does not have a Cys at position 14, or an amino acid sequence with at least 70% amino acid identity to SEQ ID NO:1 and which does not have a Cys at position 102.

According to one embodiment, one or both of the modified EHD2 domain of the third or the fourth polypeptide chain further comprises a single amino acid substitution at position N39.

According to a further embodiment, the binding molecule further comprises a fifth binding domain (BD5) and a sixth binding domain (BD6), wherein BD5 and BD6 together form an antigen binding site.

According to yet another embodiment, the binding molecule further comprises

(i) a C_{H}1 domain,

(ii) a C_{L} domain,

(iii) a first modified EHD2 domain (EHD2-1), or

(iv) a second modified EHD2 domain (EHD2-2)

connected to BD5, and

in case of (i) a C_{L} domain,

in case of (ii) a C_{H}1 domain,

in case of (iii) a second modified EHD2 domain (EHD2-2), and

in case of (iv) a first modified EHD2 domain (EHD2-1) connected to BD6, wherein the amino acid sequences of EHD2-1 and EHD2-2 are different from each other and each is selected from an amino acid sequence with at least 70% amino acid identity to SEQ ID NO:1 and which does not have a Cys at position 14, or an amino acid sequence with at least 70% amino acid identity to SEQ ID NO:1 and which does not have a Cys at position 102.

According to one embodiment, one or both of the modified EHD2 domains connected to BD5 or BD6 further comprises a single amino acid substitution at position N39.

According to another embodiment, BD5 and BD6 are different from each other and each is selected from a VH and a VL or from a variable region of a TCR α-chain and a variable region of a TCR β-chain.

According to yet another embodiment, the C_{H}1 domain, the C_{L} domain, the EHD2-1 or the EHD2-2 connected to BD5 or BD6 is connected with one of BD1, BD2, BD3 or BD4 via a linker.

According to one embodiment, the binding molecule is monospecific, bispecific, or trispecific.

According to a further embodiment, the binding molecule further comprises a seventh binding domain (BD7) and an eighth binding domain (BD8), wherein BD7 and BD8 together form an antigen binding site.

According to one embodiment, the binding molecule further comprises

(i) a C_{H}1 domain,

(ii) a C_{L} domain,

(iii) a first modified EHD2 domain (EHD2-1), or

(iv) a second modified EHD2 domain (EHD2-2)

connected to BD7, and

in case of (i) a C_{L} domain,

in case of (ii) a C_{H}1 domain,

in case of (iii) a second modified EHD2 domain (EHD2-2), and

in case of (iv) a first modified EHD2 domain (EHD2-1) connected to BD8, wherein the amino acid sequences of EHD2-1 and EHD2-2 are different from each other and each is selected from an amino acid sequence with at least 70% amino acid identity to SEQ ID NO:1 and which does not have a Cys at position 14, or an amino acid sequence with at least 70% amino acid identity to SEQ ID NO:1 and which does not have a Cys at position 102.

According to yet another embodiment, one or both of the modified EHD2 domains connected to BD7 or BD8 further comprises a single amino acid substitution at position N39.

According to one embodiment, BD7 and BD8 are different from each other and each is selected from a V_{H} and a V_{L} or from a variable region of a TCR α-chain and a variable region of a TCR β-chain.

According to a further embodiment, the C_{H}1 domain, the C_{L} domain, the EHD2-1 or the EHD2-2 connected to BD7 or BD8 is connected with one of BD1, BD2, BD3 or BD4 not connected with BD5 or BD6 via a linker.

According to yet another embodiment, the binding molecule is monospecific, bispecific, trispecific or tetraspecific.

According to a general embodiment, none, one or more of the modified EHD2 domains carries one N-glycan.

According to a further general embodiment, the Cys at position 14 of SEQ ID NO:1 is substituted by an amino acid selected from the group consisting of Ser, Gly, Ala, Thr, Gln, Asn, and Tyr (C145, C14G, C14A, C14T, C14Q, C14N, C14Y), preferably by Ser (C145).

According to a further general embodiment, the Cys at position 102 is substituted by an amino acid selected from the group consisting of Ser, Gly, Ala, Thr, Gln, Asn, and Tyr (C102S, C102G, C102A, C102T, C102Q, C102N, C102Y), preferably by Ser (C102S).

According to yet another general embodiment, the single amino acid substitution at position N39 is N39Q.

According to a further aspect, the present invention provides a nucleic acid or set of nucleic acids encoding the binding molecule of the present invention.

According to a further aspect, the present invention provides a vector comprising the nucleic acid or set of nucleic acids of the present invention.

According to a further aspect, the present invention provides a host cell comprising the vector of the present invention.

According to a further aspect, the present invention provides a pharmaceutical composition comprising the binding molecule, the nucleic acid or set of nucleic acids, the vector, or the host cell of the present invention, and a pharmaceutically acceptable carrier.

Further aspects and embodiments will become apparent from the following detailed description of the invention.

### LIST OF FIGURES

In the following, the content of the figures comprised in this specification is described. In this context please also refer to the detailed description of the invention above and/or below.

FIG. 1: Sequence (A) and structure (B) of the human wild-type EHD2 domain with the interchain disulfide bond (C14-C102) and intrachain disulfide bonds (C28-C86) as well as the

N-glycosylation site (N39) marked.

FIG. 2: Schematic scheme showing the structure of the binding molecule according to one embodiment of the invention.

FIG. 3: Examples of antibody fragments and Ig-like molecules using the hetEHD2 domains to generate molecules of varying valency and specificity.

FIG. 4: The modified EHD2 domains (EHD2-1, EHD2-2) can either carry N-glycans in both domains, only one of the domains, or can completely lack N-glycans.

FIG. 5: Biochemical characterization and binding of bispecific, bivalent eIgG molecules, specific for HER3 and MET. A Schematic illustration of both heavy chains and both light chains of the two bispecific, bivalent eIgG antibodies. B Schematic structure of the domains in both eIgG antibodies (glycan is presented as pentagon; hetEHD2=EHD2-1 (=EHD2(C14S, N39Q)) and EHD2-2 (=EHD2(C102S))). Both eIgG molecules show different glycosylation of the different hetEHD2 domains. C SDS-PAGE analysis (12% PAA; Coomassie stained) of the bispecific, bivalent eIgG antibodies under reducing (R) and non-reducing (NR) conditions (M: marker). D Size exclusion chromatography of the eIgG antibodies. E Binding of the bispecific, bivalent eIgG antibodies was analyzed by ELISA using fusion proteins of the extracellular domain of HER3 (His-tagged) or MET (Fc fusion protein) as antigen. Bound protein was detected with an HRP-conjugated anti-human Fc antibody using HER3-His as antigen and with an HRP-conjugated anti-human Fab antibody using MET-Fc as antigen. Optical density was measured at 450 nm. Mean±SD.

FIG. 6: Biochemical characterization and binding of bispecific, bivalent eIgG molecules, specific for HER3 and CD3. A Schematic illustration of both heavy chains and both light chains of the bispecific, bivalent eIgG antibody. B Schematic structure of the domains in the eIgG antibody (glycan is presented as pentagon; hetEHD2=EHD2-1 (=EHD2(C14S, N39Q)) and EHD2-2 (=EHD2(C102S))). C SDS-PAGE analysis (10% PAA; Coomassie stained) of the bispecific, bivalent eIgG antibody under reducing (R) and non-reducing (NR) conditions (M: marker). D Size exclusion chromatography of the eIgG antibody. E Binding of the bispecific, bivalent eIgG antibody was analyzed by ELISA using fusion proteins of the extracellular domain of HER3 (His-tagged) or CD3 (Fc fusion protein) as antigen. Bound protein was detected with an HRP-conjugated anti-human Fc antibody using HER3-His as antigen and with an HRP-conjugated anti-human Fab antibody using CD3-Fc as antigen. Optical density was measured at 450 nm. Mean±SD. F Flow cytometry of bispecific eIgG antibody using HER3-expressing LIM1215 cells and CD3-expressing Jurkat cells. Bound antibody was detected with PE-labeled anti-human Fc antibody. Mean±SD.

FIG. 7: eFab molecules with different residues in the position C14 of chain A and C102 in chain B of the hetEHD2 domain. (A) Composition of the eFab molecules indicating the different substitutions at the hetEHD2 domains. (B) SDS-PAGE analysis of the different eFab molecules under reducing and non-reducing conditions. (C) Binding of the different Fv3-43-hetEHD2 fusion proteins to immobilized antigen HER3-Fc in ELISA experiments. Bound Fv3-43-hetEHD2 fusion proteins were detected with anti-His detection antibodies. Mean±SD, n=1.

FIG. 8: Size-exclusion chromatograms using different Fv3-43-EHD2 molecules. Different residues were introduced at the positions C14 of chain A and C102 in chain B of an anti-HER3 eFab (see FIG. 7) and purified proteins were analyzed by size-exclusion chromatography by HPLC using a Tosoh TSKgel SuperSW mAb HR column.

FIG. 9: Analysis of Fc3-43-hetEHD2 molecules with different N-glycosylation sites. (A) Schematic composition of the eFab molecules with different N-glycosylation sites. (B) SDS-PAGE under non-reduced conditions using a 12% PAA gel. (C) ELISA experiment of the different Fv3-43-hetEHD2 molecules (100 nM) with different N-glycosylation sites. HER3-Fc was used as immobilized antigen and bound molecules were detected with an anti-His detection antibody. Mean±SD, n=1.

FIG. 10: Schematic illustration and biochemical characterization of bispecific, bi- or trivalent eIg molecules. (A) Molecular composition and schematic assembly of the Ig-like eIg and eIg-Fab molecules. N-glycans in one of the hetEHD2 domains are shown as black hexagons. (B) SDS PAGE analysis (12% PAA, 3 μg/lane, Coomassie blue staining) of eIg molecules under reducing (R) and non-reducing (NR) condition. M, protein marker. (C) Size-exclusion chromatography by HPLC using a Tosoh TSKgel SuperSW mAb HR column.

FIG. 11: Binding properties of eIg molecules. Binding to HER3-expressing LIM1215 (A), BT474 (B), and CD3-expressing Jurkat cells (C) was analyzed by flow cytometry. Bound protein was detected using a PE-labeled anti-human Fc mAb. Mean±SD, n=3.

FIG. 12: Effect of eIg and eIg-Fab molecules on cytotoxic potential of PBMCs. Target cells ((A) LIM1215, (B) BT474 cells) were incubated with a serial dilution of eIg and eIg-Fab molecules followed by addition of PBMCs in an effector:target cell ratio (E:T) of 10:1. After incubation for 3 days, crystal violet staining was used to determine cell viability. Mean±SD, n=3.

FIG. 13: Composition and biochemical analysis of the eIg antibody targeting HER3 and FAP. Composition (A) and schematic illustration (B) of the eIg molecule. HC, heavy chain; LC, light chain. (C) SDS PAGE analysis (12% PAA, 3 μg/lane, Coomassie blue staining) of eIg molecule under reducing (R) and non-reducing (NR) condition. M, protein marker. (D) Size-exclusion chromatography by HPLC using a Tosoh TSKgel SuperSW mAb HR column. (E) ELISA of the eIg molecule using HER3-His and FAP-Flag molecule as immobilized antigens. Bound eIg antibodies were detected with an anti-human Fc detection antibody. Mean±SD, n=1.

FIG. 14: Composition and biochemical analysis of the eIg antibody targeting HER3. Composition (A) and schematic illustration (B) of the eIg molecule. HC, heavy chain; LC, light chain. (C) SDS PAGE analysis (12% PAA, 3 μg/lane, Coomassie blue staining) of eIg molecule under reducing (R) and non-reducing (NR) condition. M, protein marker. (D) ELISA of eIg molecule using HER3-His molecule as immobilized antigen. Bound eIg antibodies were detected with an anti-human Fc detection antibody. Mean±SD, n=1.

FIG. 15: Composition and biochemical analysis of the eIg-Fab antibody targeting EGFR and HER3. Composition (A) and schematic illustration (B) of the eIg-Fab molecule. HC, heavy chain; LC, light chain. (C) SDS PAGE analysis (12% PAA, 3 μg/lane, Coomassie blue staining) of eIg molecule under reducing (R) and non-reducing (NR) condition. M, protein marker. (D) Size-exclusion chromatography by HPLC using a Tosoh TSKgel SuperSW mAb HR column. (E) ELISA of eIg-Fab molecule using EGFR-His or HER3-His molecule as immobilized antigen. Bound eIg antibodies were detected with an anti-human Fc detection antibody. Bifunctional binding was analyzed with immobilized EGFR-Fc antigen, a titration of eIg-Fab molecule and HER3-His as second antigen. Bound HER3-His was detected with an anti-His antibody. The parental antibodies (IgG hu225 and IgG 3-43) were included as control. Mean±SD, n=3.

FIG. 16: Composition and biochemical analysis of the Fab-eFab antibody targeting EGFR and HER3. Composition (A) and schematic illustration (B) of the Fab-eFab molecule. HC, heavy chain; LC, light chain. (C) SDS PAGE analysis (12% PAA, 3 μg/lane, Coomassie blue staining) of Fab-eFab molecule under reducing (R) and non-reducing (NR) condition. M, protein marker. (D) Size-exclusion chromatography by HPLC using a Tosoh TSKgel SuperSW mAb HR column. (E) ELISA of Fab-eFab molecule using EGFR-His or HER3-His molecule as immobilized antigen. Bound Fab-eFab antibodies or the parental antibodies were detected with an anti-human Fab antibody. Bifunctional binding was analyzed with immobilized HER3-His antigen, a titration of Fab-eFab molecule and EGFR-moFc as second antigen. Bound EGFR-moFc was detected with an anti-murine Fc antibody. The parental antibodies (IgG hu225 and IgG 3-43) were included as control. Mean±SD, n=1.

FIG. 17: Composition and biochemical analysis of the eIg antibody targeting FAP and murine CD3. Composition (A) and schematic illustration (B) of the eIg molecules. HC, heavy chain; LC, light chain. (C) SDS PAGE analysis (12% PAA, 3 μg/lane, Coomassie blue staining) of both eIg molecules under reducing (R) and non-reducing (NR) condition. M, protein marker. (D) Size-exclusion chromatography by HPLC using a Tosoh TSKgel SuperSW mAb HR column. (E) Flow cytometry analysis of both eIg molecules using FAP-expressing HT1080-FAP or murine CD3-expressing murine spleenocytes. Bound eIg antibodies were detected with a PE-conjugated anti-human Fc antibody. Mean±SD, n=1.

FIG. 18: Lack of binding of eIg molecules to human FccRT. Various eIg derivatives and IgG control antibodies were analyzed for binding to immobilized FccRT and target antigens by ELISA. 50 nM of the antibody molecules was used and detection was performed with an HRP-conjugated anti-human Fab antibody recognizing the Fab arms in IgE, the eIg derivatives and the IgG antibodies.

FIG. 19: Composition and biochemical analysis of the eIg antibody targeting RBD of the spike protein. Composition (A) and schematic illustration (B) of the eIg-Fab molecule. HC, heavy chain; LC, light chain. (C) SDS PAGE analysis (12% PAA, 3 μg/lane, Coomassie blue staining) of the eIg-Fab molecule under reducing (R) and non-reducing (NR) condition. M, protein marker. (D) Size-exclusion chromatography by HPLC using a Tosoh TSKgel SuperSW mAb HR column. (E) ELISA of eIg-Fab molecule using RBD-His molecule as immobilized antigen. Bound eIg-Fab antibodies were detected with an anti-human Fc detection antibody. Mean±SD, n=1.

List of Sequences

SEQ ID NO: 1
(wt human EHD2 core amino acid sequence; positions C14, N39 and Cl02 are underlined)
DFTPPTVKIL QSSCDGGGHF PPTIQLLCLV SGYTPGTINI TWLEDGQVMD
VDLSTASTTQ EGELASTQSE LTLSQKHWLS DRTYTCQVTY QGHTFEDSTK
KCADSN
SEQ ID NO: 2
(modified EHD2 amino acid sequence with a substitution of the Cys at position 14; X = any
amino acid but Cys, preferably Ser, Gly, Ala, Thr, Gln, Asn, or Tyr, most preferably Ser)
DFTPPTVKIL QSS [symbol] DGGGHF PPTIQLLCLV SGYTPGTINI TWLEDGQVMD
VDLSTASTTQ EGELASTQSE LTLSQKHWLS DRTYTCQVTY QGHTFEDSTK
KCADSN
SEQ ID NO: 3
(modified EHD2 amino acid sequence with a substitution of the Cys at position 102; X = any
amino acid but Cys, preferably Ser, Gly, Ala, Thr, Gln, Asn, or Tyr, most preferably Ser)
DFTPPTVKIL QSSCDGGGHF PPTIQLLCLV SGYTPGTINI TWLEDGQVMD
VDLSTASTTQ EGELASTQSE LTLSQKHWLS DRTYTCQVTY QGHTFEDSTK
K [symbol] ADSN
SEQ ID NO: 4
(modified EHD2 amino acid sequence comprising Ser at position 14)
DFTPPTVKIL QSS [symbol] DGGGHF PPTIQLLCLV SGYTPGTINI TWLEDGQVMD
VDLSTASTTQ EGELASTQSE LTLSQKHWLS DRTYTCQVTY QGHTFEDSTK
KCADSN
SEQ ID NO: 5
(modified EHD2 amino acid sequence comprising Ser at position 14 and Gln at position 39)
DFTPPTVKIL QSS [symbol] DGGGHF PPTIQLLCLV SGYTPGTI [symbol] I TWLEDGQVMD
VDLSTASTTQ EGELASTQSE LTLSQKHWLS DRTYTCQVTY QGHTFEDSTK
KCADSN
SEQ ID NO: 6
(modified EHD2 amino acid sequence comprising Ser at position 102)
DFTPPTVKIL QSSCDGGGHF PPTIQLLCLV SGYTPGTINI TWLEDGQVMD
VDLSTASTTQ EGELASTQSE LTLSQKHWLS DRTYTCQVTY QGHTFEDSTK K [symbol] ADSN
SEQ ID NO: 7
(modified EHD2 amino acid sequence comprising Ser at position 102 and Gln at position 39)
DFTPPTVKIL QSSCDGGGHF PPTIQLLCLV SGYTPGTI [symbol] I TWLEDGQVMD
VDLSTASTTQ EGELASTQSE LTLSQKHWLS DRTYTCQVTY QGHTFEDSTK K [symbol] ADSN
SEQ ID NO: 8
(IgK leader sequence)
METDTLLLWVLLLWVPGSTG
SEQ ID NO: 9
(V_{L}5D5-EHD2-1 (N39Q))
DIQMTQSPSSLSASVGDRVTITCKSSQSLLYTSSQKNYLAWYQQKPGKAPKLLIYWA
STRESGVPSRFSGSGSGTDFTLTISSLQPEDFATYYCQQYYAYPWTFGQGTKVEIKRT
DFTPPTVKILQSSSDGGGHFPPTIQLLCLVSGYTPGTIQITWLEDGQVMDVDLSTASTT
QEGELASTQSELTLSQKHWLSDRTYTCQVTYQGHTFEDSTKKCADSN
SEQ ID NO: 10
(V_{H}5D5-EHD2-2-Fcₕₒₗₑ)
EVQLVESGGGLVQPGGSLRLSCAASGYTFTSYWLHWVRQAPGKGLEWVGMIDPSNS
DTRFNPNFKDRFTISADTSKNTAYLQMNSLRAEDTAVYYCATYRSYVTPLDYWGQG
TLVTVSSDFTPPTVKILQSSCDGGGHFPPTIQLLCLVSGYTPGTINITWLEDGQVMDVD
LSTASTTQEGELASTQSELTLSQKHWLSDRTYTCQVTYQGHTFEDSTKKSADSNGTD
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKT
ISKAKGQPREPQVCTLPPSRDELTKNQVSLSCAVKGFYPSDIAVEWESNGQPENNYKT
TPPVLDSDGSFFLVSKLTVDKSRWQQGNVFSCSVMHEALHNHYTQKSLSLSPGK
SEQ ID NO: 11
(V_{L}5D5-EHD2-2)
DIQMTQSPSSLSASVGDRVTITCKSSQSLLYTSSQKNYLAWYQQKPGKAPKLLIYWA
STRESGVPSRFSGSGSGTDFTLTISSLQPEDFATYYCQQYYAYPWTFGQGTKVEIKRT
DFTPPTVKILQSSCDGGGHFPPTIQLLCLVSGYTPGTINITWLEDGQVMDVDLSTASTT
QEGELASTQSELTLSQKHWLSDRTYTCQVTYQGHTFEDSTKKSADSN
SEQ ID NO: 12
(V_{H}5D5-EHD2-1(N39Q)-Fcₕₒₗₑ)
EVQLVESGGGLVQPGGSLRLSCAASGYTFTSYWLHWVRQAPGKGLEWVGMIDPSNS
DTRFNPNFKDRFTISADTSKNTAYLQMNSLRAEDTAVYYCATYRSYVTPLDYWGQG
TLVTVSSDFTPPTVKILQSSSDGGGHFPPTIQLLCLVSGYTPGTIQITWLEDGQVMDVD
LSTASTTQEGELASTQSELTLSQKHWLSDRTYTCQVTYQGHTFEDSTKKCADSNGTD
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKT
ISKAKGQPREPQVCTLPPSRDELTKNQVSLSCAVKGFYPSDIAVEWESNGQPENNYKT
TPPVLDSDGSFFLVSKLTVDKSRWQQGNVFSCSVMHEALHNHYTQKSLSLSPGK
SEQ ID NO: 13
(V_{L}3-43-C_{L}λ)
QAGLTQPPAVSVAPGQTASITCGRDNIGSRSVHWYQQKPGQAPVLVVYDDSDRPAGI
PERFSGSNYENTATLTISRVEAGDEADYYCQVWGITSDHVVFGGGTKLTVLGQPKAA
PSVTLFPPSSEELQANKATLVCLISDFYPGAVTVAWKADSSPVKAGVETTTPSKQSNN
KYAASSYLSLTPEQWKSHRSYSCQVTHEGSTVEKTVAPTECS
SEQ ID NO: 14
(V_{H}3-43-C_{H}1-Fc_{knob})
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCGTD
KTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKT
ISKAKGQPREPQVYTLPPCRDELTKNQVSLWCLVKGFYPSDIAVEWESNGQPENNYK
TTPPVLDSDGSFFLYSKLTVDKSRWQQGNVFSCSVMHEALHNHYTQKSLSLSPGK
SEQ ID NO: 15
(V_{L}huU3-EHD2-1(N39Q))
DIQMTQSPSSLSASVGDRVTITCRASQDIRNYLNWYQQKPGKAPKLLIYYTSRLHSGV
PSRFSGSGSGTDFTFTISSLQPEDIATYYCQQGNTLPWTFGQGTKLEIKRTDFTPPTVKI
LQSSSDGGGHFPPTIQLLCLVSGYTPGTIQITWLEDGQVMDVDLSTASTTQEGELAST
QSELTLSQKHWLSDRTYTCQVTYQGHTFEDSTKKCADSN
SEQ ID NO: 16
(V_{H}huU3-EHD2-2-Fcₕₒₗₑ)
QVQLVQSGAEVKKPGSSVKVSCKASGGTFSGYTMNWVRQAPGQGLEWMGLINPYK
GVSTYNGKFKDRVTITADKSTSTAYMELSSLRSEDTAVYYCARSGYYGDSDWYFDV
WGQGTLVTVSSDFTPPTVKILQSSCDGGGHFPPTIQLLCLVSGYTPGTINITWLEDGQV
MDVDLSTASTTQEGELASTQSELTLSQKHWLSDRTYTCQVTYQGHTFEDSTKKSADS
NGTDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALP
APIEKTISKAKGQPREPQVCTLPPSRDELTKNQVSLSCAVKGFYPSDIAVEWESNGQP
ENNYKTTPPVLDSDGSFFLVSKLTVDKSRWQQGNVFSCSVMHEALHNHYTQKSLSL
SPGK
SEQ ID NO: 17
(V_{H}5D5-EHD2-2-Linker-V_{H}3-43-C_{H}1)
EVQLVESGGGLVQPGGSLRLSCAASGYTFTSYWLHWVRQAPGKGLEWVGMIDPSNS
DTRFNPNFKDRFTISADTSKNTAYLQMNSLRAEDTAVYYCATYRSYVTPLDYWGQG
TLVTVSSDFTPPTVKILQSSCDGGGHFPPTIQLLCLVSGYTPGTINITWLEDGQVMDVD
LSTASTTQEGELASTQSELTLSQKHWLSDRTYTCQVTYQGHTFEDSTKKSADSNGTG
GSGGGGSGGQVQLQQSGPGLVKPSQTLSLTCAISGDSVSSNRAAWNWIRQSPSRGLE
WLGRTYYRSKWYNDYAQSLKSRITINPDTPKNQFSLQLNSVTPEDTAVYYCARDGQ
LGLDALDIWGQGTMVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVD
KKVEPKSC
SEQ ID NO: 18
(V_{H}3-43-C_{H}1-Linker-V_{H}5D5-EHD2-2)
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCGTG
GSGGGGSGGEVQLVESGGGLVQPGGSLRLSCAASGYTFTSYWLHWVRQAPGKGLE
WVGMIDPSNSDTRFNPNFKDRFTISADTSKNTAYLQMNSLRAEDTAVYYCATYRSY
VTPLDYWGQGTLVTVSSDFTPPTVKILQSSCDGGGHFPPTIQLLCLVSGYTPGTINITW
LEDGQVMDVDLSTASTTQEGELASTQSELTLSQKHWLSDRTYTCQVTYQGHTFEDS
TKKSADSN
SEQ ID NO: 19
(V_{H}3-43-C_{H}1-Linker-V_{H}5D5-EHD2-2-Fcₕₒₗₑ)
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCGTG
GSGGGGSGGEVQLVESGGGLVQPGGSLRLSCAASGYTFTSYWLHWVRQAPGKGLE
WVGMIDPSNSDTRFNPNFKDRFTISADTSKNTAYLQMNSLRAEDTAVYYCATYRSY
VTPLDYWGQGTLVTVSSDFTPPTVKILQSSCDGGGHFPPTIQLLCLVSGYTPGTINITW
LEDGQVMDVDLSTASTTQEGELASTQSELTLSQKHWLSDRTYTCQVTYQGHTFEDS
TKKSADSNGTDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCK
VSNKALPAPIEKTISKAKGQPREPQVCTLPPSRDELTKNQVSLSCAVKGFYPSDIAVE
WESNGQPENNYKTTPPVLDSDGSFFLVSKLTVDKSRWQQGNVFSCSVMHEALHNHY
TQKSLSLSPGK
SEQ ID NO: 20
(V_{H}3-43-C_{H}1-Linker-V_{H}3-43-CH₁-Fc_{knob})
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCGTG
GSGGGGSGGQVQLQQSGPGLVKPSQTLSLTCAISGDSVSSNRAAWNWIRQSPSRGLE
WLGRTYYRSKWYNDYAQSLKSRITINPDTPKNQFSLQLNSVTPEDTAVYYCARDGQ
LGLDALDIWGQGTMVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVD
KKVEPKSCGTDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCK
VSNKALPAPIEKTISKAKGQPREPQVYTLPPCRDELTKNQVSLWCLVKGFYPSDIAVE
WESNGQPENNYKTTPPVLDSDGSFFLYSKLTVDKSRWQQGNVFSCSVMHEALHNHY
TQKSLSLSPGK
SEQ ID NO: 21
(V_{H}5D5-EHD2-2-Linker-V_{H}3-43-C_{H}1-Fcₕₒₗₑ)
EVQLVESGGGLVQPGGSLRLSCAASGYTFTSYWLHWVRQAPGKGLEWVGMIDPSNS
DTRFNPNFKDRFTISADTSKNTAYLQMNSLRAEDTAVYYCATYRSYVTPLDYWGQG
TLVTVSSDFTPPTVKILQSSCDGGGHFPPTIQLLCLVSGYTPGTINITWLEDGQVMDVD
LSTASTTQEGELASTQSELTLSQKHWLSDRTYTCQVTYQGHTFEDSTKKSADSNGTG
GSGGGGSGGQVQLQQSGPGLVKPSQTLSLTCAISGDSVSSNRAAWNWIRQSPSRGLE
WLGRTYYRSKWYNDYAQSLKSRITINPDTPKNQFSLQLNSVTPEDTAVYYCARDGQ
LGLDALDIWGQGTMVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVD
KKVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVS
NKALPAPIEKTISKAKGQPREPQVCTLPPSRDELTKNQVSLSCAVKGFYPSDIAVEWE
SNGQPENNYKTTPPVLDSDGSFFLVSKLTVDKSRWQQGNVFSCSVMHEALHNHYTQ
KSLSLSPGK
SEQ ID NO: 22
(V_{H}3-43-EHD2-2-Fc)
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSDFTPPTVKILQSSCDGGGHFPPTIQLLCLVSGYTPGTINITWLEDGQVM
DVDLSTASTTQEGELASTQSELTLSQKHWLSDRTYTCQVTYQGHTFEDSTKKSADSN
GTDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSP
GK
SEQ ID NO: 23
(V_{L}3-43-EHD2-1(N39Q))
QAGLTQPPAVSVAPGQTASITCGRDNIGSRSVHWYQQKPGQAPVLVVYDDSDRPAGI
PERFSGSNYENTATLTISRVEAGDEADYYCQVWGITSDHVVFGGGTKLTVLGTDFTP
PTVKILQSSSDGGGHFPPTIQLLCLVSGYTPGTIQITWLEDGQVMDVDLSTASTTQEGE
LASTQSELTLSQKHWLSDRTYTCQVTYQGHTFEDSTKKCADSN
SEQ ID NO: 24
(V_{H}hu225-C_{H}1-Linker-V_{H}3-43-EHD2-2-Fc)
EVQLVESGGGLVQPGGSLRLSCAASGFSLTNYGVHWVRQAPGKGLEWLGVIWSGG
NTDYNTPFTSRFTISRDNSKNTLYLQMNSLRAEDTAVYYCARALTYYDYEFAYWGQ
GTTVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEPKSCGGSGG
GGSGGQVQLQQSGPGLVKPSQTLSLTCAISGDSVSSNRAAWNWIRQSPSRGLEWLGR
TYYRSKWYNDYAQSLKSRITINPDTPKNQFSLQLNSVTPEDTAVYYCARDGQLGLDA
LDIWGQGTMVTVSSDFTPPTVKILQSSCDGGGHFPPTIQLLCLVSGYTPGTINITWLED
GQVMDVDLSTASTTQEGELASTQSELTLSQKHWLSDRTYTCQVTYQGHTFEDSTKK
SADSNGTDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQK
SLSLSPGK
SEQ ID NO: 25
(V_{L}hu225-C_{L}k)
DIQLTQSPSFLSASVGDRVTITCRASQSIGTNIHWYQQKPGKAPKLLIKYASESISGVPS
RFSGSGSGTEFTLTISSLQPEDFATYYCQQNNNWPTTFGAGTKLEIKRTVAAPSVFIFP
PSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 26
(V_{H}3-43-EHD2-2-Linker-V_{H}hu225-C_{H}1-Fc)
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSDFTPPTVKILQSSCDGGGHFPPTIQLLCLVSGYTPGTINITWLEDGQVM
DVDLSTASTTQEGELASTQSELTLSQKHWLSDRTYTCQVTYQGHTFEDSTKKSADSN
GTGGSGGGGSGGEVQLVESGGGLVQPGGSLRLSCAASGFSLTNYGVHWVRQAPGK
GLEWLGVIWSGGNTDYNTPFTSRFTISRDNSKNTLYLQMNSLRAEDTAVYYCARALT
YYDYEFAYWGQGTTVTVSSASTKGPSVFPLAPSSKSTSGGTAALGCLVKDYFPEPVT
VSWNSGALTSGVHTFPAVLQSSGLYSLSSVVTVPSSSLGTQTYICNVNHKPSNTKVD
KRVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQ
KSLSLSPGK
SEQ ID NO: 27
(V_{H}3-43-EHD2-2(C102A)-His)
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSDFTPPTVKILQSSCDGGGHFPPTIQLLCLVSGYTPGTINITWLEDGQVM
DVDLSTASTTQEGELASTQSELTLSQKHWLSDRTYTCQVTYQGHTFEDSTKK [symbol] ADSN
AAAHHHHHH
SEQ ID NO: 28
(V_{H}3-43-EHD2-2(C102W)-His)
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSDFTPPTVKILQSSCDGGGHFPPTIQLLCLVSGYTPGTINITWLEDGQVM
DVDLSTASTTQEGELASTQSELTLSQKHWLSDRTYTCQVTYQGHTFEDSTKK [symbol] ADS
NAAAHHHHHH
SEQ ID NO: 29
(V_{H}3-43-EHD2-2(C102N)-His)
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSDFTPPTVKILQSSCDGGGHFPPTIQLLCLVSGYTPGTINITWLEDGQVM
DVDLSTASTTQEGELASTQSELTLSQKHWLSDRTYTCQVTYQGHTFEDSTKK [symbol] ADSN
AAAHHHHHH
SEQ ID NO: 30
(V_{H}3-43-EHD2-2(C102T)-His)
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSDFTPPTVKILQSSCDGGGHFPPTIQLLCLVSGYTPGTINITWLEDGQVM
DVDLSTASTTQEGELASTQSELTLSQKHWLSDRTYTCQVTYQGHTFEDSTKK [symbol] ADSN
AAAHHHHHH
SEQ ID NO: 31
(V_{H}3-43-EHD2-2(C102S)-His)
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSDFTPPTVKILQSSCDGGGHFPPTIQLLCLVSGYTPGTINITWLEDGQVM
DVDLSTASTTQEGELASTQSELTLSQKHWLSDRTYTCQVTYQGHTFEDSTKK [symbol] ADSN
AAAHHHHHH
SEQ ID NO: 32
(V_{L}3-43-EHD2-1(C14A, N39Q))
QAGLTQPPAVSVAPGQTASITCGRDNIGSRSVHWYQQKPGQAPVLVVYDDSDRPAGI
PERFSGSNYENTATLTISRVEAGDEADYYCQVWGITSDHVVFGGGTKLTVLDFTPPT
VKILQSS [symbol] DGGGHFPPTIQLLCLVSGYTPGTI [symbol] ITWLEDGQVMDVDLSTASTTQEGEL
ASTQSELTLSQKHWLSDRTYTCQVTYQGHTFEDSTKKCADSN
SEQ ID NO: 33
(V_{L}3-43-EHD2-1(C14T, N39Q))
QAGLTQPPAVSVAPGQTASITCGRDNIGSRSVHWYQQKPGQAPVLVVYDDSDRPAGI
PERFSGSNYENTATLTISRVEAGDEADYYCQVWGITSDHVVFGGGTKLTVLDFTPPT
VKILQSS [symbol] DGGGHFPPTIQLLCLVSGYTPGTI [symbol] ITWLEDGQVMDVDLSTASTTQEGEL
ASTQSELTLSQKHWLSDRTYTCQVTYQGHTFEDSTKKCADSN
SEQ ID NO: 34
(V_{L}3-43-EHD2-1(C14N, N39Q))
QAGLTQPPAVSVAPGQTASITCGRDNIGSRSVHWYQQKPGQAPVLVVYDDSDRPAGI
PERFSGSNYENTATLTISRVEAGDEADYYCQVWGITSDHVVFGGGTKLTVLDFTPPT
VKILQSS [symbol] DGGGHFPPTIQLLCLVSGYTPGTI [symbol] ITWLEDGQVMDVDLSTASTTQEGEL
ASTQSELTLSQKHWLSDRTYTCQVTYQGHTFEDSTKKCADSN
SEQ ID NO: 35
(V_{L}3-43-EHD2-1(C14W, N39Q))
QAGLTQPPAVSVAPGQTASITCGRDNIGSRSVHWYQQKPGQAPVLVVYDDSDRPAGI
PERFSGSNYENTATLTISRVEAGDEADYYCQVWGITSDHVVFGGGTKLTVLDFTPPT
VKILQSS [symbol] DGGGHFPPTIQLLCLVSGYTPGTI [symbol] ITWLEDGQVMDVDLSTASTTQEGEL
ASTQSELTLSQKHWLSDRTYTCQVTYQGHTFEDSTKKCADSN
SEQ ID NO: 36
(V_{L}3-43-EHD2-1(C14S, N39Q))
QAGLTQPPAVSVAPGQTASITCGRDNIGSRSVHWYQQKPGQAPVLVVYDDSDRPAGI
PERFSGSNYENTATLTISRVEAGDEADYYCQVWGITSDHVVFGGGTKLTVLDFTPPT
VKILQSS [symbol] DGGGHFPPTIQLLCLVSGYTPGTI [symbol] ITWLEDGQVMDVDLSTASTTQEGEL
ASTQSELTLSQKHWLSDRTYTCQVTYQGHTFEDSTKKCADSN
SEQ ID NO: 37
(V_{L}3-43-EHD2-1(C14S))
QAGLTQPPAVSVAPGQTASITCGRDNIGSRSVHWYQQKPGQAPVLVVYDDSDRPAGI
PERFSGSNYENTATLTISRVEAGDEADYYCQVWGITSDHVVFGGGTKLTVLDFTPPT
VKILQSS [symbol] DGGGHFPPTIQLLCLVSGYTPGTINITWLEDGQVMDVDLSTASTTQEGEL
ASTQSELTLSQKHWLSDRTYTCQVTYQGHTFEDSTKKCADSN
SEQ ID NO: 38
(V_{H}3-43-EHD2-2(N39Q, C102S)-His)
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSDFTPPTVKILQSSCDGGGHFPPTIQLLCLVSGYTPGTI [symbol] ITWLEDGQVM
DVDLSTASTTQEGELASTQSELTLSQKHWLSDRTYTCQVTYQGHTFEDSTKK [symbol] ADSN
AAAHHHHHH
SEQ ID NO: 39
(V_{H}huU3-EHD2-2-Linker-V_{H}3-43-C_{H}1-Fc_{knob})
QVQLVQSGAEVKKPGSSVKVSCKASGGTFSGYTMNWVRQAPGQGLEWMGLINPYK
GVSTYNGKFKDRVTITADKSTSTAYMELSSLRSEDTAVYYCARSGYYGDSDWYFDV
WGQGTLVTVSSDFTPPTVKILQSSCDGGGHFPPTIQLLCLVSGYTPGTINITWLEDGQV
MDVDLSTASTTQEGELASTQSELTLSQKHWLSDRTYTCQVTYQGHTFEDSTKKSADS
NGGSGGGGSGGQVQLQQSGPGLVKPSQTLSLTCAISGDSVSSNRAAWNWIRQSPSRG
LEWLGRTYYRSKWYNDYAQSLKSRITINPDTPKNQFSLQLNSVTPEDTAVYYCARDG
QLGLDALDIWGQGTMVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVD
KKVEPKSCGTDKTHTCPPCPAPPVAGPSVFLFPPKPKDTLMISRTPEVTCVVVDVSHE
DPEVKFNWYVDGVEVHNAKTKPREEQYNSTYRVVSVLTVLHQDWLNGKEYKCKV
SNKGLPSSIEKTISKAKGQPREPQVYTLPPCRDELTKNQVSLWCLVKGFYPSDIAVEW
ESNGQPENNYKTTPPVLDSDGSFFLYSKLTVDKSRWQQGNVFSCSVMHEALHNHYT
QKSLSLSPGK
SEQ ID NO: 40
(V_{H}3-43-C_{H}1-Fcₕₒₗₑ)
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCGTD
KTHTCPPCPAPPVAGPSVFLFPPKPKDTLMISRTPEVTCVVVDVSHEDPEVKFNWYVD
GVEVHNAKTKPREEQYNSTYRVVSVLTVLHQDWLNGKEYKCKVSNKGLPSSIEKTIS
KAKGQPREPQVCTLPPSRDELTKNQVSLSCAVKGFYPSDIAVEWESNGQPENNYKTT
PPVLDSDGSFFLVSKLTVDKSRWQQGNVFSCSVMHEALHNHYTQKSLSLSPGK
SEQ ID NO: 41
(V_{H}3-43-C_{H}1-Linker-V_{H}huU3-EHD2-2- Fcₕₒₗₑ)
QVQLQQSGPGLVKPSQTLSLTCAISGDSVSSNRAAWNWIRQSPSRGLEWLGRTYYRS
KWYNDYAQSLKSRITINPDTPKNQFSLQLNSVTPEDTAVYYCARDGQLGLDALDIWG
QGTMVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCGGS
GGGGSGGQVQLVQSGAEVKKPGSSVKVSCKASGGTFSGYTMNWVRQAPGQGLEW
MGLINPYKGVSTYNGKFKDRVTITADKSTSTAYMELSSLRSEDTAVYYCARSGYYG
DSDWYFDVWGQGTLVTVSSDFTPPTVKILQSSCDGGGHFPPTIQLLCLVSGYTPGTINI
TWLEDGQVMDVDLSTASTTQEGELASTQSELTLSQKHWLSDRTYTCQVTYQGHTFE
DSTKKSADSNGTDKTHTCPPCPAPPVAGPSVFLFPPKPKDTLMISRTPEVTCVVVDVS
HEDPEVKFNWYVDGVEVHNAKTKPREEQYNSTYRVVSVLTVLHQDWLNGKEYKC
KVSNKGLPSSIEKTISKAKGQPREPQVCTLPPSRDELTKNQVSLSCAVKGFYPSDIAVE
WESNGQPENNYKTTPPVLDSDGSFFLVSKLTVDKSRWQQGNVFSCSVMHEALHNHY
TQKSLSLSPGK
SEQ ID NO: 42
(V_{H}hu36-EHD2-2-Fcₕₒₗₑ)
QVQLVQSGAEVKKPGASVKVSCKASGYTFTENIIHWVRQAPGQGLEWMGWFHPGS
GSIKYNEKFKDRVTMTADTSTSTVYMELSSLRSEDTAVYYCARHGGTGRGAMDYW
GQGTLVTVSSDFTPPTVKILQSSCDGGGHFPPTIQLLCLVSGYTPGTINITWLEDGQVM
DVDLSTASTTQEGELASTQSELTLSQKHWLSDRTYTCQVTYQGHTFEDSTKKSADSN
GTDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPA
PIEKTISKAKGQPREPQVCTLPPSRDELTKNQVSLSCAVKGFYPSDIAVEWESNGQPE
NNYKTTPPVLDSDGSFFLVSKLTVDKSRWQQGNVFSCSVMHEALHNHYTQKSLSLSP
GK
SEQ ID NO: 43
(VLhu36-EHD2-1(N39Q))
DIQMTQSPSSLSASVGDRVTITCRASKSVSTSAYSYMHWYQQKPGKAPKLLIYLASN
LESGVPSRFSGSGSGTDFTLTISSLQPEDFATYYCQHSRELPYTFGQGTKLEIKRDFTPP
TVKILQSSSDGGGHFPPTIQLLCLVSGYTPGTIQITWLEDGQVMDVDLSTASTTQEGEL
ASTQSELTLSQKHWLSDRTYTCQVTYQGHTFEDSTKKCADSN
SEQ ID NO: 44
(V_{H}hu225-C_{H}1-linker-V_{H}3-43-EHD2-2-His)
EVQLVESGGGLVQPGGSLRLSCAASGFSLTNYGVHWVRQAPGKGLEWLGVIWSGG
NTDYNTPFTSRFTISRDNSKNTLYLQMNSLRAEDTAVYYCARALTYYDYEFAYWGQ
GTTVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEPKSCGGSGG
GGSGGQVQLQQSGPGLVKPSQTLSLTCAISGDSVSSNRAAWNWIRQSPSRGLEWLGR
TYYRSKWYNDYAQSLKSRITINPDTPKNQFSLQLNSVTPEDTAVYYCARDGQLGLDA
LDIWGQGTMVTVSSDFTPPTVKILQSSCDGGGHFPPTIQLLCLVSGYTPGTINITWLED
GQVMDVDLSTASTTQEGELASTQSELTLSQKHWLSDRTYTCQVTYQGHTFEDSTKK
SADSNAAAHHHHHH
SEQ ID NO: 45
(V_{L}hu36-C_{L}K)
DIQMTQSPSSLSASVGDRVTITCRASKSVSTSAYSYMHWYQQKPGKAPKLLIYLASN
LESGVPSRFSGSGSGTDFTLTISSLQPEDFATYYCQHSRELPYTFGQGTKLEIKRRTVA
APSVFIFPPSDEQLKSGTASVVCLLNNFYPREAKVQWKVDNALQSGNSQESVTEQDS
KDSTYSLSSTLTLSKADYEKHKVYACEVTHQGLSSPVTKSFNRGEC
SEQ ID NO: 46
(V_{H}hu36-C_{H}1-Fcₕₒₗₑ)
QVQLVQSGAEVKKPGASVKVSCKASGYTFTENIIHWVRQAPGQGLEWMGWFHPGS
GSIKYNEKFKDRVTMTADTSTSTVYMELSSLRSEDTAVYYCARHGGTGRGAMDYW
GQGTLVTVSSASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCGTD
KTHTCPPCPAPPVAGPSVFLFPPKPKDTLMISRTPEVTCVVVDVSHEDPEVKFNWYVD
GVEVHNAKTKPREEQYNSTYRVVSVLTVLHQDWLNGKEYKCKVSNKGLPSSIEKTIS
KAKGQPREPQVCTLPPSRDELTKNQVSLSCAVKGFYPSDIAVEWESNGQPENNYKTT
PPVLDSDGSFFLVSKLTVDKSRWQQGNVFSCSVMHEALHNHYTQKSLSLSPGK
SEQ ID NO: 47
(V_{H}2C11-EHD2-1(N39Q))
EVQLVESGGGLVQPGKSLKLSCEASGFTFSGYGMHWVRQAPGRGLESVAYITSSSINI
KYADAVKGRFTVSRDNAKNLLFLQMNILKSEDTAMYYCARFDWDKNYWGQGTMV
TVSSDFTPPTVKILQSSSDGGGHFPPTIQLLCLVSGYTPGTIQITWLEDGQVMDVDLST
ASTTQEGELASTQSELTLSQKHWLSDRTYTCQVTYQGHTFEDSTKKCADSN
SEQ ID NO: 48
(V_{L}2C11-EHD2-2-Fc_{knob})
DIQMTQSPSSLPASLGDRVTINCQASQDISNYLNWYQQKPGKAPKLLIYYTNKLADG
VPSRFSGSGSGRDSSFTISSLESEDIGSYYCQQYYNYPWTFGPGTKLEIKDFTPPTVKIL
QSSCDGGGHFPPTIQLLCLVSGYTPGTINITWLEDGQVMDVDLSTASTTQEGELASTQ
SELTLSQKHWLSDRTYTCQVTYQGHTFEDSTKKSADSNGTDKTHTCPPCPAPPVAGP
SVFLFPPKPKDTLMISRTPEVTCVVVDVSHEDPEVKFNWYVDGVEVHNAKTKPREEQ
YNSTYRVVSVLTVLHQDWLNGKEYKCKVSNKGLPSSIEKTISKAKGQPREPQVYTLP
PCRDELTKNQVSLWCLVKGFYPSDIAVEWESNGQPENNYKTTPPVLDSDGSFFLYSK
LTVDKSRWQQGNVFSCSVMHEALHNHYTQKSLSLSPGK
SEQ ID NO: 49
(V_{L}2C11-EHD2-1(N39Q))
DIQMTQSPSSLPASLGDRVTINCQASQDISNYLNWYQQKPGKAPKLLIYYTNKLADG
VPSRFSGSGSGRDSSFTISSLESEDIGSYYCQQYYNYPWTFGPGTKLEIKDFTPPTVKIL
QSSSDGGGHFPPTIQLLCLVSGYTPGTIQITWLEDGQVMDVDLSTASTTQEGELASTQ
SELTLSQKHWLSDRTYTCQVTYQGHTFEDSTKKCADSN
SEQ ID NO: 50
(V_{H}2C11-EHD2-2-Fc_{knob})
EVQLVESGGGLVQPGKSLKLSCEASGFTFSGYGMHWVRQAPGRGLESVAYITSSSINI
KYADAVKGRFTVSRDNAKNLLFLQMNILKSEDTAMYYCARFDWDKNYWGQGTMV
TVSSDFTPPTVKILQSSCDGGGHFPPTIQLLCLVSGYTPGTINITWLEDGQVMDVDLST
ASTTQEGELASTQSELTLSQKHWLSDRTYTCQVTYQGHTFEDSTKKSADSNGTDKTH
TCPPCPAPPVAGPSVFLFPPKPKDTLMISRTPEVTCVVVDVSHEDPEVKFNWYVDGVE
VHNAKTKPREEQYNSTYRVVSVLTVLHQDWLNGKEYKCKVSNKGLPSSIEKTISKA
KGQPREPQVYTLPPCRDELTKNQVSLWCLVKGFYPSDIAVEWESNGQPENNYKTTPP
VLDSDGSFFLYSKLTVDKSRWQQGNVFSCSVMHEALHNHYTQKSLSLSPGK
SEQ ID NO: 51
(V_{L}S309-C_{L}K)
EIVLTQSPGTLSLSPGERATLSCRASQTVSSTSLAWYQQKPGQAPRLLIYGASSRATGI
PDRFSGSGSGTDFTLTISRLEPEDFAVYYCQQHDTSLTFGGGTKVEIKRTVAAPSVFIF
PPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 52
(V_{L}P2B-2F6-EHD2-1(N39Q))
QSALTQPPSASGSPGQSVTISCTGTSSDVGGYNYVSWYQQHPGKAPKLMIYEVSKRP
SGVPDRFSGSKSGNTASLTVSGLQAEDEADYYCSSYAGSNNLVCGGGTKLTVLDFTP
PTVKILQSSSDGGGHFPPTIQLLCLVSGYTPGTIQITWLEDGQVMDVDLSTASTTQEGE
LASTQSELTLSQKHWLSDRTYTCQVTYQGHTFEDSTKKCADSN
SEQ ID NO: 53
(V_{H}S309-C_{H}1-V_{H}P2B-2F6-EHD2-2-Fc)
QVQLVQSGAEVKKPGASVKVSCKASGYPFTSYGISWVRQAPGQGLEWMGWISTYN
GNTNYAQKFQGRVTMTTDTSTTTGYMELRRLRSDDTAVYYCARDYTRGAWFGESLI
GGFDNWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDKKVE
PKSCGGSGGGGSGGQVQLQESGPGLVKPSETLSLTCTVSGYSISSGYYWGWIRQPPG
KGLEWIGSIYHSGSTYYNPSLKTRVTISVDTSKNQFSLKLSSVTAADTAVYYCARAVV
GIVVVPAAGRRAFDIWGQGTMVTVSSDFTPPTVKILQSSCDGGGHFPPTIQLLCLVSG
YTPGTINITWLEDGQVMDVDLSTASTTQEGELASTQSELTLSQKHWLSDRTYTCQVT
YQGHTFEDSTKKSADSNDKTHTCPPCPAPPVAGPSVFLFPPKPKDTLMISRTPEVTCV
VVDVSHEDPEVKFNWYVDGVEVHNAKTKPREEQYNSTYRVVSVLTVLHQDWLNG
KEYKCKVSNKGLPSSIEKTISKAKGQPREPQVYTLPPSRDELTKNQVSLTCLVKGFYP
SDIAVEWESNGQPENNYKTTPPVLDSDGSFFLYSKLTVDKSRWQQGNVFSCSVMHE
ALHNHYTQKSLSLSPGK

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. In particular, any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The term “binding molecule”, as used herein, refers to any molecule or part of a molecule that can specifically bind to a target molecule or target epitope. The binding properties of the binding molecule of the present invention can be derived from (a) antibodies or antigen-binding fragments thereof; (b) oligonucleotides; (c) antibody-like proteins; d) a T-cell receptor or (e) peptidomimetics.

The term “binding” according to the invention preferably relates to a specific binding. “Specific binding” means that a binding protein (e.g. an antibody) binds stronger to a target such as an epitope for which it is specific compared to the binding to another target. A binding protein binds stronger to a first target compared to a second target if it binds to the first target with a dissociation constant (K_{d}) which is lower than the dissociation constant for the second target. Preferably the dissociation constant (K_{d}) for the target to which the binding protein binds specifically is more than 10-fold, preferably more than 20-fold, more preferably more than 50-fold, even more preferably more than 100-fold, 200-fold, 500-fold or 1000-fold lower than the dissociation constant (K_{d}) for the target to which the binding protein does not bind specifically.

As used herein, the term “K_{d}” (measured in “mol/L”, sometimes abbreviated as “M”) is intended to refer to the dissociation equilibrium constant of the particular interaction between a binding protein (e.g. an antibody or fragment thereof) and a target molecule (e.g. an antigen or epitope thereof). Methods for determining binding affinities of compounds, i.e. for determining the dissociation constant K_{D}, are known to a person of ordinary skill in the art and can be selected for instance from the following methods known in the art: Surface Plasmon Resonance (SPR) based technology, Bio-layer interferometry (BLI), quartz crystal microbalance (QCM), enzyme-linked immunosorbent assay (ELISA), flow cytometry, isothermal titration calorimetry (ITC), analytical ultracentrifugation, radioimmunoassay (MA or IRMA) and enhanced chemiluminescence (ECL). In the context of the present application, the “K_{d}” value is determined by surface plasmon resonance spectroscopy (Biacore™) or by quartz crystal microbalance (QCM) at room temperature (25° C.).

The term “binding domain” as used herein refers to a sequence of amino acids having the ability to specifically bind to an antigen and can be derived, for example, from antibodies or antigen binding fragments thereof and T-cell receptors and antigen-binding fragments thereof. Examples encompassed within the term “binding domain” include but are not limited to Fab fragments, monovalent fragments consisting of the V_{L}, V_{H} domains; Fv fragments consisting of the V_{L} and V_{H} domains of a single arm of an antibody, dAb fragments (Ward et al., (1989) Nature 341: 544-546), which consist of a V_{H} domain or a V_{L} domain, a VHH, a Nanobody, or a variable domain of an IgNAR; isolated complementarity determining regions (CDR), and combinations of two or more isolated CDRs which may optionally be joined by a synthetic peptide linker. The term “binding domain” also refers to the variable domain or region of a TCR α- and a TCR β-chain or of a TCR γ-chain and a TCR δ-chain.

As used herein, the term “TCR” or “T cell receptor” denotes a molecule found on the surface of T cells. The TCR is composed of two separate peptide chains, which are in most cases in humans produced from the independent T cell receptor alpha and beta (TCRα and TCRβ) genes. These chains are called α- and β-chains. Each of the TCR α- and the TCR β-chain has a variable and a constant domain. Each variable domain carries three CDRs for binding to an antigen. TCR γ- and TCR δ-chains are less abundant in human but also carry a variable and a constant domain, with each variable domain carrying three CDRs for binding to an antigen.

The term “antigen” as used herein relates to an agent comprising an epitope which is recognized by an antigen binding domain. The term “antigen” includes in particular proteins and peptides. An antigen is preferably a product which corresponds to or is derived from a naturally occurring antigen. Such naturally occurring antigens may include or may be derived from allergens, viruses, bacteria, fungi, parasites and other infectious agents and pathogens or an antigen may also be a tumor antigen. According to the present invention, an antigen may correspond to a naturally occurring product, for example, a viral protein, or a part thereof, or a tumor protein.

The term “dimerization domain” as used herein refers to a domain capable of forming a dimer of two polypeptide or protein chains, wherein at least one dimerization domain is present on the first chain and at least a second dimerization domain is present on the second chain. A dimerization domain can be selected from the group consisting of an Fc region, a heterodimerizing Fc region, C_{H}1 C_{L}, the second heavy chain constant domain (C_{H}2) of IgE and IgM (EHD2, MHD2), modified EHD2 according to the present invention, the last heavy chain constant domain (C_{H}3 or C_{H}4) of IgG, IgD, IgA, IgM, or IgE and heterodimerizing derivatives thereof, and the constant domains C-α and C-β of a T-cell receptor. Depending on the respective dimerization domain, the C-terminus and N-terminus of the dimerization domain may vary. If the dimerization domain is derived from a naturally occurring protein, e.g. an immunoglobulin, the dimerization domain can be, preferably, directly linked to the variable domain in the sense of the present invention, i.e. linked without a peptide linker, if there are no non-naturally occurring amino acids at its C- or N-terminus.

The V_{H} and V_{L} domains as used in the context of the present invention are preferably antibody or immunoglobulin derived. Such “antibody” or “immunoglobulin” may be a natural or conventional antibody having a “Y”-shaped form and consisting of four polypeptide chains; two identical heavy chains and two identical light chains connected by disulfide bonds. Each chain is composed of structural domains. Two heavy chains are linked to each other by disulfide bonds and each heavy chain is linked to a light chain by a disulfide bond. There are two types of light chains, lambda (λ) and kappa (k), and five main heavy chain classes (or isotypes) which determine the functional activity of an antibody molecule: IgM, IgD, IgG, IgA and IgE. Each chain contains distinct sequence domains. The light chain includes two domains or regions, a variable domain (V_{L}) and a constant domain (C_{L}). The heavy chain includes four domains, a variable domain (V_{H}) and three constant domains (CH1, CH2 and CH3, collectively referred to as CH), or in case of IgE and IgM five domains, a variable domain (V_{H}) and four constant domains (CH1, CH2, CH3, CH4). The variable regions of both light chains (V_{L}) and heavy chains (V_{H}) determine binding recognition and specificity to the antigen. The constant region domains of the light (C_{L}) and heavy (C_{H}) chains confer important biological properties such as antibody chain association, secretion, trans-placental mobility, complement binding, and binding to Fc receptors (FcR). The “arms” of the “Y”-shaped antibody contain the sites that can bind to specific molecules, enabling recognition of specific antigens. This region of the antibody is called the Fab (fragment, antigen-binding) region. It is composed of one constant and one variable domain from each heavy and light chain of the antibody. The base of the Y plays a role in modulating immune cell activity. This region is called the Fc (fragment, crystallizable) region, and is composed of two heavy chains that contribute two or three constant domains depending on the class of the antibody. The Fv fragment is the N-terminal part of the Fab region of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences that together define the binding affinity and specificity of the natural Fv region of a native immunoglobulin binding site. The light and heavy chains of an immunoglobulin each have three CDRs, designated CDR1-L, CDR2-L, CDR3-L and CDR1-H, CDR2-H, CDR3-H, respectively. A conventional antibody antigen-binding site, therefore, includes six CDRs, comprising the CDR set from each of a heavy and a light chain V region.

Antibodies and antigen-binding fragments thereof usable for the present invention may be from any animal origin including birds and mammals. Preferably, the antibodies or fragments are from human, chimpanzee, rodent (e.g. mouse, rat, guinea pig, or rabbit), chicken, turkey, pig, sheep, goat, camel, cow, horse, donkey, cat, or dog origin. It is particularly preferred that the antibodies are of human or murine origin. Antibodies of the invention also include chimeric molecules in which an antibody constant region derived from one species, preferably human, is combined with the antigen binding site derived from another species, e.g. mouse. Moreover, antibodies of the invention include humanized molecules in which the antigen binding sites of an antibody derived from a non-human species (e.g. from mouse) are combined with constant and framework regions of human origin.

As exemplified herein, antibodies can be obtained directly from hybridomas which express the antibody, or can be cloned and recombinantly expressed in a host cell (e.g., a CHO cell, or a lymphocytic cell). Further examples of host cells are microorganisms, such as E. coli, and fungi, such as yeast. Alternatively, they can be produced recombinantly in a transgenic non-human animal or plant.

As used herein, the term “EHD2” and “EHD2 domain” refers to the second constant domain of the heavy chain of IgE (Seifert et al., 2012, Protein Eng Des Sel.; 25:603-12; and Seifert et al., 2014, Mol Cancer Ther.;13:101-11). Likewise, the term “modified EHD2 domain” refers to a part of the EHD2 domain, which sequence has been modified compared the original EHD2 sequence from which it is derived. Modifications include amino acid deletions, substitutions, and insertions. The modification preferably includes one or more amino acid substitutions, preferably at the positions indicated with respect to SEQ ID NO:1. The substitutions according to the present invention are made for Cys at position 14 or Cys at position 102. Preferred substitutions include C14S and C102S, respectively, with respect to SEQ ID NO:1. As used herein, the term “hetEHD2” denotes a heterodimer of two modified EHD2 domains, i.e. a dimer containing EHD2-1 and EHD2-2 as defined herein. It is emphasized that although in some examples reference is made to a specific amino acid sequence, the terms “EHD2-1” and “EHD2-2” as used herein do not refer to a specific amino acid sequence and are used primarily for emphasizing a difference between the amino acid sequences of a first modified EHD2 domain and a second EHD2 domain. Using the amino acid numbering scheme defined in Lefranc et al., 2005 (Developmental and Comparative Immunology 29, 185-203) for constant immunoglobulin domains, C14 in SEQ ID NO: 1 corresponds to C11, C28 in SEQ ID NO: 1 corresponds to C23, C86 in SEQ ID NO: 1 corresponds to C104, C102 in SEQ ID NO: 1 corresponds to C124, and N39 in SEQ ID NO: 1 corresponds to N38.

The term “monoclonal antibody” as used herein refers to a preparation of antibody molecules of single molecular composition. A monoclonal antibody displays a single binding specificity and affinity for a particular epitope. In one embodiment, the monoclonal antibodies are produced by a hybridoma which includes a B cell obtained from a non-human animal, e.g. mouse, fused to an immortalized cell.

The term “recombinant antibody”, as used herein, includes all antibodies that are prepared, expressed, created or isolated by recombinant means, such as (a) antibodies isolated from an animal (e.g., a mouse) that is transgenic or transchromosomal with respect to the immunoglobulin genes or a hybridoma prepared therefrom, (b) antibodies isolated from a host cell transformed to express the antibody, e.g. from a transfectoma, (c) antibodies isolated from a recombinant, combinatorial antibody library, and (d) antibodies prepared, expressed, created or isolated by any other means that involve splicing of immunoglobulin gene sequences to other DNA sequences.

Thus, “antibodies and antigen-binding fragments thereof” suitable for use in the present invention include, but are not limited to, polyclonal, monoclonal, monovalent, bispecific, heteroconjugate, multi specific, recombinant, heterologous, heterohybrid, chimeric, humanized (in particular CDR-grafted), deimmunized, or human antibodies, Fab fragments, Fab′ fragments, F(ab′)₂ fragments, fragments produced by a Fab expression library, Fd, Fv, disulfide-linked Fvs (dsFv), single chain antibodies (e.g. scFv), diabodies or tetrabodies (Holliger P. et al. (1993) Proc. Natl. Acad. Sci. U.S.A. 90(14), 6444-6448), nanobodies (also known as single domain antibodies), anti-idiotypic (anti-Id) antibodies (including, e.g., anti-Id antibodies to antibodies of the invention), and epitope-binding fragments of any of the above.

As used herein, the term “bispecific” denotes a binding molecule, such as an antibody, that can bind to two different antigens or two different epitopes within one antigen. Likewise, the terms “trispecific” and “tetraspecific” denote binding molecules that can bind to three and four different antigens or epitopes within an antigen, respectively.

The term “naturally occurring”, as used herein, as applied to an object refers to the fact that an object can be found in nature. For example, a polypeptide or polynucleotide sequence that is present in an organism (including viruses) that can be isolated from a source in nature and which has not been intentionally modified by man in the laboratory is naturally occurring.

As used herein, the term “nucleic acid aptamer” refers to a nucleic acid molecule that has been engineered through repeated rounds of in vitro selection or SELEX (systematic evolution of ligands by exponential enrichment) to bind to a target molecule (for a review see: Brody E. N. and Gold L. (2000), Aptamers as therapeutic and diagnostic agents. J. Biotechnol. 74(1):5-13). The nucleic acid aptamer may be a DNA or RNA molecule. The aptamers may contain modifications, e.g. modified nucleotides such as 2′-fluorine-substituted pyrimidines.

As used herein, the term “antibody-like protein” refers to a protein that has been engineered (e.g. by mutagenesis of loops) to specifically bind to a target molecule. Typically, such an antibody-like protein comprises at least one variable peptide attached at both ends to a protein scaffold. This double structural constraint greatly increases the binding affinity of the antibody-like protein to levels comparable to that of an antibody. The length of the variable peptide loop typically consists of 10 to 20 amino acids. The scaffold protein may be any protein having good solubility properties. Preferably, the scaffold protein is a small globular protein. Antibody-like proteins include without limitation affibodies, anticalins, and designed ankyrin repeat proteins (for review see: Binz H. K. et al. (2005) Engineering novel binding proteins from nonimmunoglobulin domains. Nat. Biotechnol. 23(10):1257-1268). Antibody-like proteins can be derived from large libraries of mutants, e.g. be panned from large phage display libraries and can be isolated in analogy to regular antibodies. Also, antibody-like binding proteins can be obtained by combinatorial mutagenesis of surface-exposed residues in globular proteins. Antibody-like proteins are sometimes referred to as “peptide aptamers”.

The “percentage of sequences identity” is determined by comparing two optimally aligned sequences over a comparison window, wherein the portion of the sequence in the comparison window can comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” is used herein in the context of two or more nucleic acids or polypeptide sequences, to refer to two or more sequences or subsequences that are the same, i.e. comprise the same sequence of nucleotides or amino acids. Sequences are “identical” to each other if they have a specified percentage of nucleotides or amino acid residues that are the same. According to the present invention, at least 70% identical includes at least 75%, at least 80, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity over the specified sequence, when compared and aligned for maximum correspondence over a comparison window, or designated region as measured using one of the following sequence comparison algorithms or by manual alignment and visual inspection. These definitions also refer to the complement of a test sequence. Accordingly, the term “at least 70% sequence identity” is used throughout the specification with regard to polypeptide and polynucleotide sequence comparisons. This expression preferably refers to a sequence identity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

The term “sequence comparison” is used herein to refer to the process wherein one sequence acts as a reference sequence, to which test sequences are compared. When using a sequence comparison algorithm, test and reference sequences are entered into a computer, if necessary subsequence coordinates are designated, and sequence algorithm program parameters are designated. Default program parameters are commonly used, or alternative parameters can be designated. The sequence comparison algorithm then calculates the percent sequence identities for the test sequences relative to the reference sequence, based on the program parameters. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by SEQ ID, if not specifically indicated otherwise.

In a sequence alignment, the term “comparison window” refers to those stretches of contiguous positions of a sequence which are compared to a reference stretch of contiguous positions of a sequence having the same number of positions. Typically, the number of contiguous positions ranges from about 20 to 100 contiguous positions, from about 25 to 90 contiguous positions, from about 30 to 80 contiguous positions, from about 40 to about 70 contiguous positions, from about 50 to about 60 contiguous positions. According to the present invention, when comparing a sequence with a sequence of the present invention, such as SEQ ID NO:1, for percentage identity, preferably the whole length of the SEQ ID NO, such as the 106 amino acids of SEQ ID NO: 1, is to be compared with a reference sequence, if the reference sequence has the same length or is longer than the SEQ ID NO of the present invention. If the reference sequence is shorter than the SEQ ID NO of the present invention, the entire length of the reference sequence must be compared with the whole length of the SEQ ID NO of the present invention.

Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wisc.), or by manual alignment and visual inspection (see, e.g., Ausubel et al., Current Protocols in Molecular Biology (1995 supplement)). Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=-4, and a comparison of both strands. The BLAST algorithm also performs a statistical analysis of the similarity between two sequences (see, e.g., Karlin and Altschul, Proc. Natl. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, typically less than about 0.01, and more typically less than about 0.001.

The term “nucleic acid” and “nucleic acid molecule” are used synonymously herein and are understood as single or double-stranded oligo- or polymers of deoxyribonucleotide or ribonucleotide bases or both. Nucleotide monomers are composed of a nucleobase, a five-carbon sugar (such as but not limited to ribose or 2′-deoxyribose), and one to three phosphate groups. Typically, a nucleic acid is formed through phosphodiester bonds between the individual nucleotide monomers, In the context of the present invention, the term nucleic acid includes but is not limited to ribonucleic acid (RNA) and deoxyribonucleic acid (DNA) molecules but also includes synthetic forms of nucleic acids comprising other linkages (e.g., peptide nucleic acids as described in Nielsen et al. (Science 254:1497-1500, 1991). Typically, nucleic acids are single- or double-stranded molecules and are composed of naturally occuring nucleotides. The depiction of a single strand of a nucleic acid also defines (at least partially) the sequence of the complementary strand. The nucleic acid may be single or double stranded, or may contain portions of both double and single stranded sequences. Exemplified, double-stranded nucleic acid molecules can have 3′ or 5′ overhangs and as such are not required or assumed to be completely double-stranded over their entire length. The nucleic acid may be obtained by biological, biochemical or chemical synthesis methods or any of the methods known in the art, including but not limited to methods of amplification, and reverse transcription of RNA. The term nucleic acid comprises chromosomes or chromosomal segments, vectors (e.g., expression vectors), expression cassettes, naked DNA or RNA polymer, primers, probes, cDNA, genomic DNA, recombinant DNA, cRNA, mRNA, tRNA, microRNA (miRNA) or small interfering RNA (siRNA). A nucleic acid can be, e.g., single-stranded, double-stranded, or triple-stranded and is not limited to any particular length. Unless otherwise indicated, a particular nucleic acid sequence comprises or encodes complementary sequences, in addition to any sequence explicitly indicated.

The term “C-terminus” (also known as the carboxyl-terminus, carboxy-terminus, C-terminal tail, C-terminal end, or COOH-terminus) as referred to within the context of the present invention is the end of an amino acid chain (protein or polypeptide), terminated by a free carboxyl group (—COOH). When the protein is translated from messenger RNA, it is created from N-terminus to C-terminus. The term “N-terminus” (also known as the amino-terminus, NH₂-terminus, N-terminal end or amine-terminus) refers to the start of a protein or polypeptide terminated by an amino acid with a free amine group (—NH₂). The convention for writing peptide sequences is to put the N-terminus on the left and write the sequence from N- to C-terminus.

The term “linker” or “peptide linker” in the context of the present invention refers to an amino acid sequence, i.e. polypeptide, which sterically separates two parts within the engineered polypeptides of the present invention. Typically, such peptide linker consists of between 1 and 100, preferably 3 to 50 more preferably 5 to 20 amino acids. Thus, such peptide linkers have a minimum length of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids, and a maximum length of at least 100, 95, 90, 85, 80, 75, 70, 65, 60, 55, 50, 45, 40, 35, 34, 33, 32, 31, 30, 29, 28, 27, 26, 25, 24, 23, 22, 21, 20, 19, 18, 17, 16, or 15 amino acids or less. Peptide linkers may also provide flexibility among the two parts that are linked together. Such flexibility is generally increased, if the amino acids are small. Accordingly, flexible peptide linkers comprise an increased content of small amino acids, in particular of glycins and/or alanines, and/or hydrophilic amino acids such as serines, threonines, asparagines and glutamines. Preferably, more than 20%, 30%, 40%, 50%, 60% or more of the amino acids of the peptide linker are small amino acids. Preferred peptide linkers have the sequence GGGGS, [G₄S]₂, [G₄S]₃, [G₂SG₂], [G₂SG₂]₂, [G₂SG₂]_{3.} The skilled person can easily determine suitable lengths for a linker that disfavors or prevents intra-chain interaction within one polypeptide chain.

As used herein, the term “substantially the same” means a deviation of up to 10% and including 10%, or up to 15% and including 15% of the disclosed value or number. In particular, the term encompasses a deviation of 1%, 2%, 3%, 4%, 5%, 6%, 7%, 8%, 9%, 10%, 11%, 12%, 13%, 14% or 15% of the disclosed value or number.

The term, Fc chain” as used herein refers to a Fc part, which can form a homodimer or heterodimer, and binds to the respective effector molecules preferably with either increased or reduced affinity, thus altering the effector function, e.g. ADCC, CMC, or FcRn-mediated recycling. There are different IgG variants with altered interaction for human FcγRIIIa (CD16) described in literature (Presta et al., 2008, Curr Opin Immunol. 20: 460-470), e.g. IgG1-DE (S239D, I332E) resulting in 10-fold increased ADCC, or IgG1-DEL (S239D, I332E, A330L) resulting in 100-fold increased ADCC. Besides increasing the effector function, there also Fc parts with reduced effector function described in the literature. For the IgG1-P329G LALA variant (L234A, L235A, P329G) almost complete abolished interaction with the whole Fcγ receptor family was reported, resulting in effector silent molecules (Schlothauer et al., 2016, Protein Eng Des Sel. 29; 457-466). In addition, reduced binding to FcγRI, which was described for the IgG-Δab variant (E233P, L234V, L235A, Δ236G, A327G, A330S, P331S) also resulted in reduced effector function (Armour et al., 1999; Eur J Immunol. 29: 2612-2624) (also described in Strohl et al., 2009; Curr Opin Biotechnol; 20: 685-691). Besides altering binding to receptors of immune cells (e.g. human FcγRIIIa), also binding to FcRn can be altered by introducing substitutions in the Fc part. Due to increased (or reduced) binding to the FcRn molecule, half-life of the Fc-containing molecule is affected, e.g. IgG1-YTE (M252Y, S254T, T256E) resulting in 3-4 fold increased terminal half-life of the protein, or IgG1-QL (T250Q, M428L) resulting in 2.5-fold increased terminal half-life (Presto et al., 2008; Strohl et al., 2009).

The term “heterodimerizing Fc” or “heterodimeric Fc” relates to variants of a Fc part, which are able to form heterodimers. Besides the knob-into-hole technology there are other variants of the Fc part described in literature for the generation of heterodimeric Fc parts (Krah et al., 2017, N. Biotechnol. 39: 167-173; Ha et al., 2016, Front Immuno. 7: 394; Mimoto et al., 2016, Curr Pharm Biotechnol. 17: 1298-1314; Brinkmann & Kontermann, 2017, MAbs 9: 182-212). The “Knob-into-Hole” or also called “Knobs-into-Holes” technology refers to mutations Y349C, T366S, L368A and Y407V (Hole) and S354C and T366W (Knob) both in the CH3-CH3 interface to promote heteromultimer formation and has been described in patents U.S. Pat. Nos. 5,731,168 and 8,216,805, notably, both of which are herein incorporated by reference.

The dimerization domains C_{H}1 and C_{L} used in the context of the invention are based on immunoglobulins and can be derived from any class (e.g., IgG, IgE, IgM, IgD, IgA and IgY), or subclass of immunoglobulin molecule (e.g., IgG1, IgG2, IgG3, IgG4, IgA1 and IgA2).

As used herein, the term “trigger molecule on an immune effector cell” refers to molecules coupled to a receptor molecule of the immune system such as pattern recognition receptors (PRRs), Toll-like receptors (TLRs), killer activated and killer inhibitor receptors (KARs and KIRs), complement receptors, Fc receptors, B cell receptors and T cell receptors. Binding to these receptors causes a response in the immune system via the trigger molecules. Non limiting examples of trigger molecules on an immune effector cell are selected form the group consisting of but not limited to CD2, CD3, CD16, CD44, CD64, CD69, CD89, Mel14, Ly-6.2C, TCR-complex, Vy9V52 TCR, and NKG2D.

The term “pharmaceutical composition” as used herein refers to a substance and/or a combination of substances being used for the identification, prevention or treatment of a disease or tissue status. The pharmaceutical composition is formulated to be suitable for administration to a patient in order to prevent and/or treat a disease. Further a pharmaceutical composition refers to the combination of an active agent with a carrier, inert or active, making the composition suitable for therapeutic use. Pharmaceutical compositions can be formulated for oral, parenteral, topical, inhalative, rectal, sublingual, transdermal, subcutaneous or vaginal application routes according to their chemical and physical properties. Pharmaceutical compositions comprise solid, semisolid, liquid, transdermal therapeutic systems (TTS). Solid compositions are selected from the group consisting of tablets, coated tablets, powder, granulate, pellets, capsules, effervescent tablets or transdermal therapeutic systems. Also comprised are liquid compositions, selected from the group consisting of solutions, syrups, infusions, extracts, solutions for intravenous application, solutions for infusion or solutions of the carrier systems of the present invention. Semisolid compositions that can be used in the context of the invention comprise emulsion, suspension, creams, lotions, gels, globules, buccal tablets and suppositories.

The term “active agent” refers to the substance in a pharmaceutical composition or formulation that is biologically active, i.e. that provides pharmaceutical value. According to the present invention, a pharmaceutical composition comprises at least the binding molecule according to the invention as active agent. However, a pharmaceutical composition may comprise more than one active agent which may act in conjunction with or independently of each other. The active agent can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as but not limited to those derived from sodium, potassium, ammonium, calcium, ferric hydroxides, isopropylamine, triethylamine, 2-ethylamino ethanol, histidine, procaine, and the like.

The terms used herein are defined as described in “A multilingual glossary of biotechnological terms: (IUPAC Recommendations)”, H. G. W. Leuenberger, B. Nagel, and H. Kölbl, Eds., (1995) Helvetica Chimica Acta, CH-4010 Basel, Switzerland.

The practice of the present invention will employ, unless otherwise indicated, conventional methods of biochemistry, cell biology, immunology, and recombinant DNA techniques which are explained in the literature in the field (cf., e.g., Molecular Cloning: A Laboratory Manual, 2n^{d} Edition, J. Sambrook et al. eds., Cold Spring Harbor Laboratory Press, Cold Spring Harbor 1989).

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

### Description of Embodiments

In the following different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

In a first aspect, the present invention provides a binding molecule comprising a first polypeptide chain comprising a first binding domain (BD1) and a first modified EHD2 domain (EHD2-1), and a second polypeptide chain comprising a second binding domain (BD2) and a second modified EHD2 domain (EHD2-2). The amino acid sequences of EHD2-1 and EHD2-2 are different from each other. One is an amino acid sequence with at least 70% amino acid identity to SEQ ID NO:1 and does not have a Cys at position 14, and the other is an amino acid sequence with at least 70% amino acid identity to SEQ ID NO:1 and does not have a Cys at position 102. The Cys is preferably substituted by a different amino acid, preferably selected form the group consisting of Ser, Gly, Ala, Thr, Gln, Asn, and Tyr, most preferably by Ser. Substitution of Cys at position 14 or 102 of SEQ ID NO:1 prevents formation of a disulfide bridge between the amino acid at position 14 on the amino acid sequence of the first modified EHD2 domain and the amino acid at position 102 of the second modified EHD2 domain. Nevertheless, the modified EHD2 domains are still capable of forming a covalent bond and preferably one disulfide bridge between each other, thereby serving as dimerization domains for the binding domains. In the binding molecule of the present invention, binding domains BD1 and BD2 together form an antigen binding site, giving rise to the binding properties and characteristics of the binding molecule.

Thus, according to the present invention, EHD2-1 and EHD2-2 are covalently bound to each other, preferably by way of a disulfide bond. Both modified EHD2 domains (EHD2-1 and EHD2-2) preferably have the same or at least substantially the same length, i.e. the same or substantially the same number of amino acids.

Without wishing to be bound by any theory, a substitution of Cys at position 14 in one modified EHD2 domain and substitution of Cys at position 102 in the other modified EHD2 domain prevents formation of a disulfide bridge between these two positions. In natural EHD2 domains, the disulfide bonds confer a symmetry, allowing formation of homodimers. Depletion of one disulfide bond between the modified EHD2 domains gives rise to an asymmetry, allowing formation of heterodimers only. Thus, by substituting Cys at position 14 in a first modified EHD2 domain by e.g. Ser and substituting Cys at position 102 in the other modified EHD2 domain by e.g. Ser, it was surprisingly found that this heterodimeric domains can be applied to generate heterodimeric binding molecules by fusing a first binding domain to the first modified EHD2 domain (carrying the C14 substitution) in a first polypeptide chain, and a second binding domain to the second modified EHD2 domain (carrying the C102 substitution) in a second polypeptide chain. By further fusing one of the modified EHD2 domains to an Fc region it is possible to generate bi-, tri- or tetravalent molecules with varying specificities. The single amino acid substitutions in the two EHD2 domains keep the number of mutations at a minimum, which further maintains the natural interface between the two domains while forcing heterodimerization by directed or guided disulfide bond formation.

Compared to state of the art, binding molecules of the present invention using alternative EHD2 modifications (hetEHD2) have one or more of the following advantages:

The thermal stability of the heterodimers is not or at least to a lesser extend reduced, e.g. as determined by dynamic light scattering or differential scanning calorimetry.

The sensitivity against proteolytic degradation of the heterodimers is not or at least to a lesser extend reduced.

The immunogenicity of the heterodimers is not or at least to a lesser extend increased. Use of the modified EHD2 domains results in an increased fraction of heterodimers over homodimers.

The modified EHD2 domains do not interfere with cysteines of the hinge region.

It is noted that the intra-domain disulfide bond between positions C28 and C86 of SEQ ID NO: 1, giving rise to a disulfide bond within one EHD2 domain is preferably maintained in the modified EHD2 domains (EHD2-1 and EHD2-2).

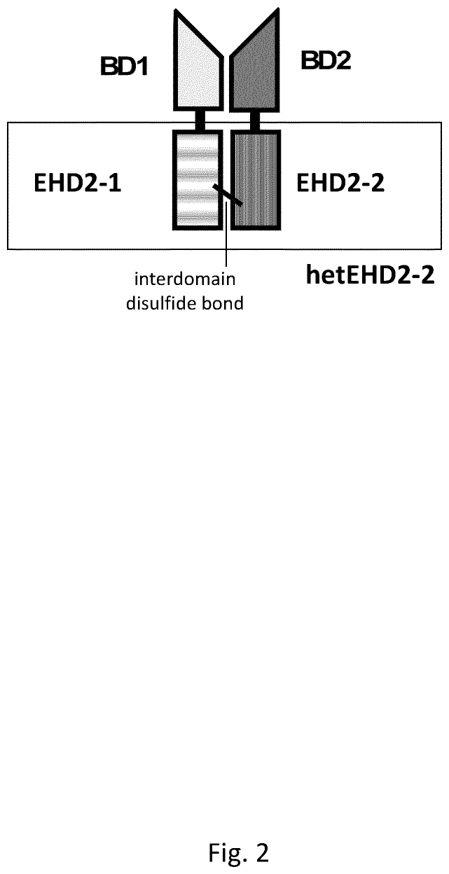

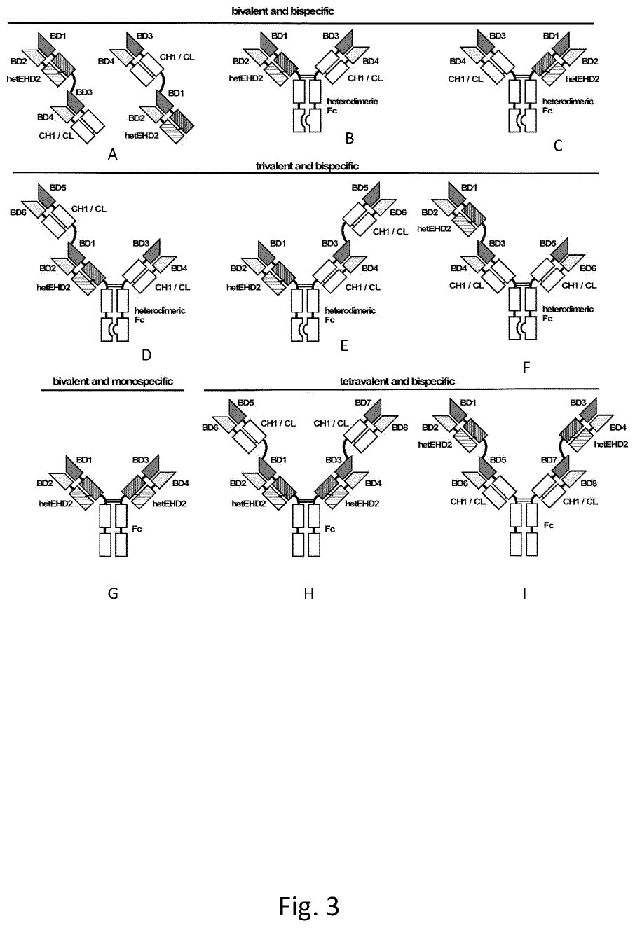

The basic structure of the binding molecule according to the present invention is schematically depicted in FIG. 2 with binding domain BD1 being connected to EHD2-1 and binding domain BD2 being connected to EHD2-2. Both modified EHD2 domains together form the heterodimeric EHD2 denoted as hetEHD2.

According to a preferred embodiment, the Cys at position 14 is preferably substituted by an amino acid selected from the group consisting of Ser, Gly, Ala, Thr, Gln, Asn, and Tyr (C14S, C14G, C14A, C14T, C14Q, C14N, and C14Y). In a most preferred embodiment of the invention, the Cys at position 14 of SEQ ID NO: 1 is substituted by Ser (C145).

According to a further preferred embodiment, the Cys at position 102 is preferably substituted by an amino acid selected from the group consisting of Ser, Gly, Ala, Thr, Gln, Asn, and Tyr (C102S, C102G, C102A, C102T, C102Q, C102N, C102Y). In a most preferred embodiment of the invention, the Cys at position 102 of SEQ ID NO: 1 is substituted by Ser (C102S). Although the substitutions recited above can be combined in any way, the most preferred set of mutations is C14S in one modified EHD2 domain and C102S in the other modified EHD2 domain. Thus, according to one particularly preferred embodiment, one of the modified EHD2 domains (EHD2-1 and EHD2-2) contains the substitution C14S and the other one of the modified EHD2 domains contains the substitution C102S.

It is noted that although each of the modified EHD2 domains (EHD2-1 and EHD2-2) may have at least 70% amino acid identity to SEQ ID NO: 1, it is preferred that the amino acid sequences of both modified EHD2 domains are substantially the same with respect to the amino acid sequence disregarding the specified substitutions at positions 14 and 102, respectively.

It is further noted that a sequence at least 70% identical to SEQ ID NO: 1 in all cases contains the specified substitution, such as substitutions at positions C14 and C102, respectively. It is further preferred that the cysteine residues at positions 28 and 86 are maintained and are not subject to any mutations such as single amino acid substitutions.

According to a preferred embodiment of the invention, one or both of the modified EHD2 domains further comprises a single amino acid substitution at position N39. A preferred substitute for the Asn at this position is Gln (N39Q).

According to a further preferred embodiment, the binding modules BD1 and BD2 are different from each other and each is selected from a V_{H} and a V_{L}. Alternatively, BD1 and BD2 are different from each other and each is selected from a TCR α-chain and a TCR β-chain.

The binding molecule of the present invention may further comprise a first Fc chain. The Fc chain may have increased or reduced effector function. This first Fc chain is preferably connected to one of the first or second polypeptide.

According to a preferred embodiment of the present invention, the binding molecule of the invention further comprises a third binding domain (BD3) and a fourth binding domain (BD4). Both binding domains BD3 and BD4 together form an antigen binding site. This antigen binding site may be the same or may be different from the binding site formed by BD1 and BD2.

According to one embodiment, BD3 and BD4 are different from each other and each is selected from a V_{H} and a V_{L}. Alternatively, BD3 and BD4 are different from each other and each is selected from a TCR α-chain and a TCR β-chain.

The third binding domain (BD3) may be located on the same polypeptide as the first or the second binding domain (BD1, BD2). Likewise, the fourth biding domain (BD4) may be located on the same polypeptide as the first or the second binding domain (BD1, BD2), preferably on a different polypeptide than the third binding domain (BD3).

According to a further preferred embodiment, the binding molecule of the invention further comprises a second Fc chain. This second Fc chain is preferably connected to one of the third or fourth polypeptide. The Fc chains in the binding molecule of the invention allow dimerization of BD1 and BD2 with BD3 and BD4, giving rise to a bivalent binding molecule. The Fc chains may be homodimerizing Fc chains or heterodimerizing Fc chains. According to a preferred embodiment, the first and the second Fc chains are different from each other and form a heterodimeric Fc.

According to a preferred embodiment, the third polypeptide chain of the binding molecule of the invention comprising BD3 further comprises a dimerization domain selected from the group consisting of a C_{H}1 domain, a C_{L} domain, a first modified EHD2 domain (EHD2-1), and a second modified EHD2 domain (EHD2-2). Accordingly, the fourth polypeptide chain of the binding molecule of the invention comprises the respective counter binding domain selected from the group consisting of a C_{L} domain, a C_{H}1 domain, a second modified EHD2 domain (EHD2-2), and a first modified EHD2 domain (EHD2-1). In other words, in case BD3 comprises a C_{H}1 domain, BD4 comprises a C_{L} domain. In case BD3 comprises a C_{L} domain, BD4 comprises a C_{H}1 domain. In case BD3 comprises a first modified EHD2 domain (EHD2-1), BD4 comprises a second modified EHD2 domain (EHD2-2). In case BD3 comprises a second modified EHD2 domain (EHD2-2), BD4 comprises a first modified EHD2 domain (EHD2-1). The modified EHD2 domains are as defined above, i.e. the amino acid sequences of EHD2-1 and EHD2-2 are different from each other. The amino acid sequences of one of said modified EHD2 domains (EHD2-1 and EHD2-2) is an amino acid sequence with at least 70% amino acid identity to SEQ ID NO: 1 and which does not have a Cys at position 14, and the amino acid sequences of the other one of said modified EHD2 domains (EHD2-1 and EHD2-2) is an amino acid sequence with at least 70% amino acid identity to SEQ ID NO: 1 and which does not have a Cys at position 102. As defined above, the Cys at the positions indicated is preferably substituted by a different amino acid selected from the group consisting of Ser, Gly, Ala, Thr, Gln, Asn, and Tyr, most preferably by Ser.

The present invention thus encompasses bivalent binding molecules being either monospecific or bispecific.

These binding molecules comprise the first and second polypeptide chains comprising BD1 and BD2 and the modified EHD2 domains EHD2-1 and EHD2-2. In addition, these binding molecules may comprise a third and fourth polypeptide chain comprising:

(i) BD3-C_{H}1 and BD4-C_{L};

(ii) BD3-C_{L} and BD4-C_{H}1;

(iii) BD3-EHD2-1 and BD4-EHD2-2; or

(iv) BD3-EHD2-2 and BD4-EHD2-1.

Non-limiting examples for embodiment (i) and (ii) are shown in FIG. 3B and 3C. A non-limiting example for embodiments (iii) and (iv) is shown in FIG. 3G, exemplified as bivalent monospecific binding molecule. According to one exemplarily embodiment the binding molecule has the sequence as denoted in SEQ ID NO: 22+23.

For the production of bispecific binding molecules, the Fc region is preferably heterodimeric, meaning that one of the first and second polypeptide chains comprises a first Fc chain, and one of the third and fourth polypeptide chains comprises a second, different Fc chain, which form a heterodimeric Fc region in the binding molecule. Non-limiting examples of such bivalent bispecific binding molecules comprising a heterodimeric Fc region are shown in the top row of FIG. 3B and 3C.

According to one embodiment, if the third and the fourth polypeptide chains comprise modified EHD2 domains, one or both of the modified EHD2 domains further comprises a single amino acid substitution at position N39. A preferred substitute for the asparagine at this position is glutamine (N39Q).

According to the present invention, where four different polypeptide chains are expressed within one cell, using heterodimerizing polypeptide chains with one of the chains having a C_{H}1 domain and the other chain comprising a first modified EHD2 domain (EHD2-1), co-expressed with a polypeptide chain comprising a C_{L} domain and a polypeptide chain comprising the second modified EHD2 domain (EHD2-2), correct assembly of the cognate heavy and light chains is observed, resulting in bivalent, bispecific antibodies which retain full antigen binding specificity for the respective antigens.

Alternatively, the binding molecule of the present invention may comprise four binding domains on three polypeptide chains. According to a respective embodiment, two binding domains are located on a first polypeptide chain and the other two binding domains on a second and a third polypeptide chain, respectively.

Exemplary embodiments of such bivalent binding modules having three polypeptide chains are shown in FIG. 3A and may have the following polypeptide chains:

(i) BD1—(optional linker)—EHD2-2—linker—BD3—(optional linker)—C_{H}1

(ii) BD2—(optional linker)—EHD2-1

(iii) BD4—(optional linker)—C_{L}

As illustrated in FIG. 3A left, upon co-expression of all three polypeptide chains above in a cell, in the resulting binding molecule according to the invention, polypeptide chain (ii) pairs with the EHD2-1 domain of (i), and polypeptide chain (iii) pairs with C_{H}1 domain of (i). According to one exemplarily embodiment, the binding molecule has the sequence as denoted in SEQ ID NO: 9+13+17, as schematically depicted in FIG. 3A.

In an alternative embodiment, a binding molecule according to the present invention comprising three polypeptide chains may have the following polypeptide chains:

(i) BD3—(optional linker)—C_{H}1—linker—BD1—(optional linker)—EHD2-2

(ii) BD2—(optional linker)—EHD2-1

(iii) BD4—(optional linker)—C_{L}

The linker is preferably a peptide linker as defined above.

As illustrated in FIG. 3A right, upon co-expression of all three polypeptide chains above in a cell, in the resulting binding molecule according to the invention, polypeptide chain (ii) pairs with the EHD2-1 domain of (i), and polypeptide chain (iii) pairs with C_{H}1 domain of (i). According to one exemplarily embodiment the binding molecule has the sequence as denoted in SEQ ID NO: 9+13+18.

It is to be understood that the order or arrangement and selection of the dimerizing domains and of the binding domains within the tetravalent binding molecules may be changed and adapted to individual needs, as long as there is at least one pair of modified EHD2 domains, as defined herein, giving rise to a heterodimeric EHD2 domain.

According to a further embodiment of the present invention, the binding molecule is bivalent and comprises four polypeptide chains. For illustration only, exemplary embodiments of such trivalent binding molecule comprising four polypeptide chains are depicted in FIG. 3B and 3C. This binding molecule may have the following polypeptide chains:

(i) BD1—(optional linker)—EHD2-1—Fc

(ii) BD2—(optional linker)—EHD2-2

(iii) BD3—(optional linker)—C_{H}1—Fc

(iv) BD4—(optional linker)—C_{L.}

As illustrated in FIG. 3B and 3C, upon co-expression of all four polypeptide chains in a cell, the resulting binding molecule according to the invention comprises polypeptide chains (i) and (iii) as ‘heavy chains’ and polypeptide chains (ii) and (iv) as corresponding ‘light chains’, with (ii) pairing with the EHD2-1 domain of (i), and (iv) pairing with the C_{H}1 domain of (iii). According to one exemplarily embodiment the binding molecule has the sequence as denoted in SEQ ID NO: 9+10+13+14, as is schematically depicted in FIG. 3B.

According to a further embodiment of the present invention, the binding molecule is trivalent and thus further comprises a fifth binding domain (BD5) and a sixth binding domain (BD6). Both, binding domains BD5 and BD6 together form an antigen binding site.

According to a preferred embodiment of the present invention, the binding molecule further comprises a C_{H}1 domain, a C_{L} domain, a first modified EHD2 domain (EHD2-1), or a second modified EHD2 domain (EHD2-2) connected to BD5. In this embodiment of the invention, BD6 is connected to the respective counterpart selected from a C_{L} domain, a C_{H}1 domain, a second modified EHD2 domain (EHD2-2), and a first modified EHD2 domain (EHD2-1). In other words, in case BD5 is connected to a C_{H}1 domain, BD6 is connected to a C_{L} domain. In case BD5 is connected to a C_{L} domain, BD6 is connected to a C_{H}1 domain. In case BD5 is connected to a first modified EHD2 domain (EHD2-1), BD6 is connected to a second modified EHD2 domain (EHD2-2). In case BD5 is connected to a second modified EHD2 domain (EHD2-2), BD6 is connected to a first modified EHD2 domain (EHD2-1). The modified EHD2 domains are as defined above, i.e. the amino acid sequences of EHD2-1 and EHD2-2 are different from each other. The amino acid sequences of one of said modified EHD2 domains (EHD2-1 and EHD2-2) is an amino acid sequence with at least 70% amino acid identity to SEQ ID NO: 1 and which does not have a Cys at position 14, and the amino acid sequences of the other one of said modified EHD2 domains (EHD2-1 and EHD2-2) is an amino acid sequence with at least 70% amino acid identity to SEQ ID NO: 1 and which does not have a Cys at position 102. As defined above, the Cys at the positions indicated is preferably substituted by a different amino acid selected from the group consisting of Ser, Gly, Ala, Thr, Gln, Asn, and Tyr, most preferably by Ser.

As used herein, the term “connected to” means a connection between two polypeptides either by means of a direct peptide bond or via a peptide linker as defined above. Hence, according to one embodiment, the C_{H}1 domain, the C_{L} domain, the EHD2-1 or the EHD2-2 connected to BD5 or BD6 is connected with one of BD1, BD2, BD3 or BD4 via a linker.

According to one embodiment, one or both of the modified EHD2 domains connected to BD5 or BD6 further comprises a single amino acid substitution at position N39. A preferred substitute for the asparagine at this position is glutamine (N39Q).

According to a preferred embodiment, BD5 and BD6 are different from each other and each is selected from a V_{H} and a V_{L}. Alternatively, BD5 and BD6 are different from each other and each is selected from a TCR α-chain and a TCR β-chain.

Binding molecules according to the present invention and comprising three binding sites, i.e. being trivalent, may bind to the same or different targets, giving rise to trivalent monospecific, bispecific or even trispecific binding molecules. Preferably, in these trivalent binding molecules according to the present invention, the C_{H}1 domain, the C_{L} domain, the EHD2-1 or the EHD2-2 connected to BD5 or BD6 is connected to one of BD1, BD2, BD3 or BD4, preferably via a linker. It is emphasized that in these trivalent molecules, one or two sets of the modified EHD2 domains can be used for providing the spatial arrangement of the binding domains for forming the antigen binding sites.

The trivalent binding molecules according to the invention may thus comprise essentially three units, each one forming an antigen binding site:

(i) the first and second polypeptide chains comprising BD1 and BD2, and the modified EHD2 domains EHD2-1 and EHD2-2;

(ii) the third and the fourth polypeptide chains comprising BD3 and BD4, and either the modified EHD2 domains EHD2-1 and EHD2-2, or domains C_{H}1 and C_{L}; and

(iii) BD5 and BD6 connected to either the modified EHD2 domains EHD2-1 and EHD2-2, or to domains C_{H}1 and C_{L}.

According to a preferred embodiment of the trivalent binding molecule, one of BD5 and BD6 in combination with one of the dimerization domains EHD2-1, EHD2-2, C_{H}1 and C_{L} forms part of a further, fifth polypeptide chain. Correspondingly, the other one of BD5 and BD6 together with the corresponding dimerization domain (the other one of EHD2-1, EHD2-2, CH1, and C_{L}) connected thereto is coupled to either one of BD1, BD2, BD3 or BD4.

For illustration only, an exemplary embodiment of such trivalent binding molecule comprising five polypeptide chains is depicted in FIG. 3D. This binding molecule has the following polypeptide chains:

(i) BD5—(optional linker)—CH1—linker—BD1—(optional linker)—EHD2-1—(optional linker)—Fc

(ii) BD2—(optional linker)—EHD2-2

(iii) BD3—(optional linker)—C_{H}1—Fc

(iv) BD4—(optional linker)—C_{L}

(v) BD6—(optional linker)—C_{L}.

As illustrated in FIG. 3D, upon co-expression of all five polypeptide chains in a cell, the resulting binding molecule according to the invention comprises polypeptide chains (i) and (iii) as ‘heavy chains’ and polypeptide chains (ii), (iv) and (v) as corresponding ‘light chains’, with (ii) pairing with the EHD2-1 domain of (i), (iii) pairing with (iv), and (v) pairing with the C_{H}1 domain of (i). According to one exemplarily embodiment the binding molecule has the sequence as denoted in SEQ ID NO: 9+13+14+19, as schematically depicted in FIG. 3D.

Likewise, other constructs can be made, according to individual demands. A further non-limiting embodiment is shown in FIG. 3E. Such binding molecule according to one embodiment of the present invention also comprises five polypeptide chains:

(i) BD1—(optional linker)—EHD2-1—(optional linker)—Fc

(ii) BD2—(optional linker)—EHD2-2

(iii) BD5—(optional linker)—C_{H}1—linker—BD3—(optional linker)—C_{H}1—(optional linker)—Fc

(iv) BD4—(optional linker)—C_{L}

(v) BD6—(optional linker)—C_{L}.

As illustrated in FIG. 3E, upon co-expression of all the five polypeptide chains above in a cell, the resulting binding molecule according to the invention comprises polypeptide chains (i) and (iii) as ‘heavy chains’ and polypeptide chains (ii), (iv) and (v) as corresponding ‘light chains’, with (ii) pairing with the EHD2-1 domain of (i), (iv) pairing with the C_{H}1 domain of (iii), and (v) pairing with the C_{H}1 domain of (iii). According to one exemplarily embodiment, the binding molecule has the sequence as denoted in SEQ ID NO: 9+10+13+20, as schematically depicted in FIG. 3E.

A further non-limiting example is shown in FIG. 3F. Such binding molecule according to one embodiment of the present invention also comprises five polypeptide chains:

(i) BD1—(optional linker)—EHD2-1—linker—BD3—(optional linker)—C_{H}1—(optional linker)—Fc

(ii) BD2—(optional linker)—EHD2-2

(iii) BD5—(optional linker)—C_{H}1—(optional linker)—Fe

(iv) BD4—(optional linker)—C_{L}

(v) BD6—(optional linker)—C_{L}.

As illustrated in FIG. 3F, upon co-expression of all the five polypeptide chains above in a cell, the resulting binding molecule according to the invention comprises polypeptide chains (i) and (iii) as ‘heavy chains’ and polypeptide chains (ii), (iv) and (v) as corresponding ‘light chains’, with (ii) pairing with the EHD2-1 domain of (i), (iv) pairing with the C_{H}1 domain of (i), and (v) pairing with the C_{H}1 domain of (iii). According to one exemplarily embodiment, the binding molecule has the sequence as denoted in SEQ ID NO: 9+13+14+21, as schematically depicted in FIG. 3F.

It is emphasized that the order or arrangement and selection of the dimerizing domains and of the binding domains within the constructs may be changed and adapted to individual needs, as long as there is at least one pair of modified EHD2 domains, i.e. EHD2-1 and EHD2-2 as defined herein. Binding molecules according to the present invention may thus also have two pairs of modified EHD2 domains. Examples of such binding molecules according to the invention are not limited to trivalent binding molecules but also encompass tetravalent binding molecules.

According to a further embodiment of the present invention, the binding molecule is trivalent and thus further comprises a seventh binding domain (BD7) and an eighth binding domain (BD8). Both, binding domains BD7 and BD8 together form an antigen binding site.

According to a preferred embodiment of the present invention, the binding molecule further comprises a C_{H}1 domain, a C_{L} domain, a first modified EHD2 domain (EHD2-1), or a second modified EHD2 domain (EHD2-2) connected to BD7. In this embodiment of the invention, BD8 is connected to the respective counterpart selected from a C_{L} domain, a C_{H}1 domain, a second modified EHD2 domain (EHD2-2), and a first modified EHD2 domain (EHD2-1). In other words, in case BD7 is connected to a C_{H}1 domain, BD8 is connected to a C_{L} domain. In case BD7 is connected to a C_{L} domain, BD8 is connected to a C_{H}1 domain. In case BD7 is connected to a first modified EHD2 domain (EHD2-1), BD8 is connected to a second modified EHD2 domain (EHD2-2). In case BD7 is connected to a second modified EHD2 domain (EHD2-2), BD8 is connected to a first modified EHD2 domain (EHD2-1). The modified EHD2 domains are as defined above, i.e. the amino acid sequences of EHD2-1 and EHD2-2 are different from each other. The amino acid sequences of one of said modified EHD2 domains (EHD2-1 and EHD2-2) is an amino acid sequence with at least 70% amino acid identity to SEQ ID NO: 1 and which does not have a Cys at position 14, and the amino acid sequences of the other one of said modified EHD2 domains (EHD2-1 and EHD2-2) is an amino acid sequence with at least 70% amino acid identity to SEQ ID NO: 1 and which does not have a Cys at position 102. As defined above, the Cys at the positions indicated is preferably substituted by a different amino acid selected from the group consisting of Ser, Gly, Ala, Thr, Gln, Asn, and Tyr, most preferably by Ser.

As used herein, the term “connected to” means a connection between two polypeptides either by means of a direct peptide bond or via a peptide linker as defined above. Hence, according to one embodiment, the C_{H}1 domain, the C_{L} domain, the EHD2-1 or the EHD2-2 connected to BD7 or BD8 is connected with one of BD1, BD2, BD3, BD4, BD5, or BD6 via a linker.

According to one embodiment, one or both of the modified EHD2 domains connected to BD7 or BD8 further comprises a single amino acid substitution at position N39. A preferred substitute for the asparagine at this position is glutamine (N39Q).

According to a preferred embodiment, BD7 and BD8 are different from each other and each is selected from a VH and a VL. Alternatively, BD7 and BD8 are different from each other and each is selected from a TCR α-chain and a TCR β-chain.

Binding molecules according to the present invention and comprising four binding sites, i.e. being tetravalent, may bind to the same or different targets, giving rise to tetravalent monospecific, bispecific, trispecific or even tetraspecific binding molecules. Preferably, in these tetravalent binding molecules according to the present invention, the C_{H}1 domain, the C_{L} domain, the EHD2-1 or the EHD2-2 connected to BD7 or BD8 is connected to one of BD1, BD2, BD3, BD4, BD5 or BD6 preferably via a linker. It is emphasized that in these tetravalent molecules, one or two sets of the modified EHD2 domains can be used for providing the spatial arrangement of the binding domains for forming the antigen binding sites.

The tetravalent binding molecules according to the invention may thus comprise essentially four units, each one forming an antigen binding site:

(i) the first and second polypeptide chains comprising BD1 and BD2, and the modified EHD2 domains EHD2-1 and EHD2-2;

(ii) the third and the fourth polypeptide chains comprising BD3 and BD4, and either the modified EHD2 domains EHD2-1 and EHD2-2, or domains C_{H}1 and C_{L};

(iii) BD5 and BD6 connected to either the modified EHD2 domains EHD2-1 and EHD2-2, or to domains C_{H}1 and C_{L}; and

(iv) BD7 and BD8 connected to either the modified EHD2 domains EHD2-1 and EHD2-2, or to domains C_{H}1 and C_{L}.

According to a preferred embodiment of the tetravalent binding molecule, one of BD7 and BD8 in combination with one of the dimerization domains EHD2-1, EHD2-2, C_{H}1 and C_{L} forms part of a further, sixth polypeptide chain. Correspondingly, the other one of BD7 and BD8 together with the corresponding dimerization domain (the other one of EHD2-1, EHD2-2, C_{H}1, and C_{L}) connected thereto is coupled to either one of BD1, BD2, BD3, BD4, BD5 or BD6.

Non-limiting examples of tetravalent binding molecules according to the present invention are shown in FIG. 3H and 3I. A non-limiting embodiment of a tetravalent binding molecule according to the present invention may comprise six polypeptide chains. A non-limiting embodiment of such binding molecule may have the following polypeptide chains:

(i) BD5—(optional linker)—C_{H}1 —linker—BD1—(optional linker)—EHD2-1—(optional linker)—Fc

(ii) BD2—(optional linker)—EHD2-2

(iii) BD7—(optional linker)—C_{H}1 —linker—BD3—(optional linker)—EHD2-1—(optional linker)—Fc

(iv) BD4—(optional linker)—EHD2-2

(v) BD6—(optional linker)—C_{L}

(vi) BD8—(optional linker)—C_{L}.

As illustrated in FIG. 3H, upon co-expression of all the six polypeptide chains above in a cell, the resulting binding molecule according to the invention comprises polypeptide chains (i) and (iii) as ‘heavy chains’ and polypeptide chains (ii), (iv), (v) and (vi) as corresponding ‘light chains’, with (ii) pairing with the EHD2-1 domain of (i), (iv) pairing with the EHD2-1 domain of (iii), (v) pairing with the C_{H}1 domain of (i), and (vi) pairing with the C_{H}1 domain of (iii). According to one exemplarily embodiment, the binding molecule has the sequence as denoted in SEQ ID NO: 23+24+25, as schematically depicted in FIG. 3H.

A further non-limiting example of a tetravalent binding molecule according to the present invention is shown in FIG. 31. The non-limiting embodiment of such binding molecule may have the following six polypeptide chains:

(i) BD1—(optional linker)—EHD2-1—linker—BD5—(optional linker)—C_{H}1—(optional linker)—Fc

(ii) BD2—(optional linker)—EHD2-2

(iii) BD3—(optional linker)—EHD2-1—linker—BD7—(optional linker)—C_{H}1—(optional linker)—Fc

(iv) BD4—(optional linker)—EHD2-2

(v) BD6—(optional linker)—C_{L}

(vi) BD8—(optional linker)—C_{L}.

As illustrated in FIG. 31, upon co-expression of all the six polypeptide chains above in a cell, the resulting binding molecule according to the invention comprises polypeptide chains (i) and (iii) as ‘heavy chains’ and polypeptide chains (ii), (iv), (v) and (vi) as corresponding ‘light chains’, with (ii) pairing with the EHD2-1 domain of (i), (iv) pairing with the EHD2-1 domain of (iii), (v) pairing with the C_{H}1 domain of (i), and (vi) pairing with the C_{H}1 domain of (iii). According to one exemplarily embodiment, the binding molecule has the sequences as denoted in SEQ ID NO: 23+25+26, as schematically depicted in FIG. 31.

It will be appreciated that—as laid out above for the trivalent binding molecules according to the present invention—the order or arrangement and selection of the dimerizing domains and the binding domains within the tetravalent binding molecules may be changed and adapted to individual needs, as long as there is at least two pairs of modified EHD2 domains, as defined herein.

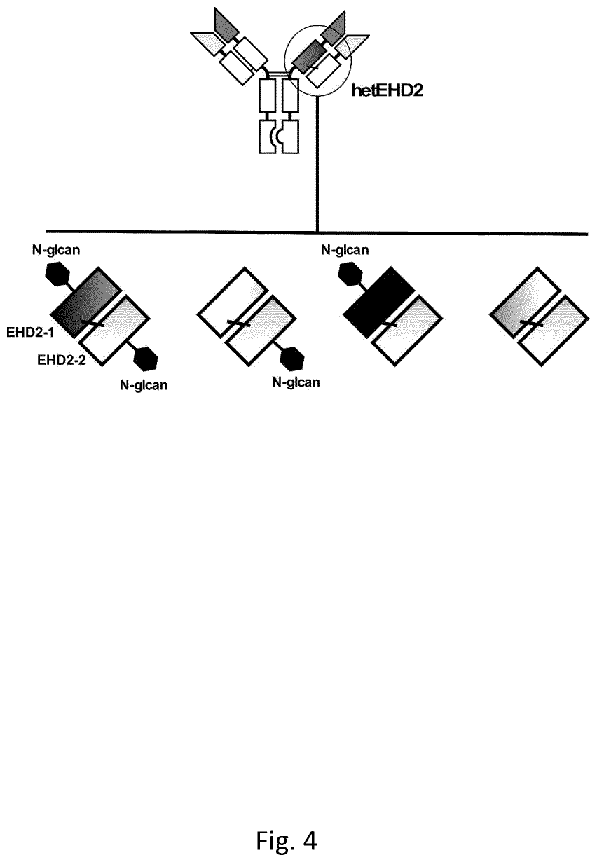

According to one embodiment of the present invention, in the binding molecule of the invention, one or more of the modified EHD2 domains carries one N-glycan. This can be achieved for example by substituting a further amino acid preferably at position N39. A preferred amino acid substitution is substituting glutamine for asparagine at this position (N39Q). FIG. 4 schematically and exemplarily shows the modified EHD2 domains (EHD2-1, EHD2-2) to carry N-glycans in both domains (EHD2-1 and EHD2-2), in only one of the domains (EHD2-1 or EHD2-2), or to completely lack N-glycans.

It is again emphasized that the Cys substitution required in the modified EHD2 domain is either at position 14 or at position 102 of SEQ ID NO: 1. The Cys at the respective position 14 and 102 can be substituted by any amino acid. The substitute amino acid can be the same or can be different for both positions 14 and 102. According to a preferred embodiment, a first modified EHD2 domain comprises a substitution selected from the group consisting of C14S, C14G, C14A, C14T, C14Q, C14N, C14Y, preferably C14S. According to this embodiment, the second modified EHD2 domain comprises a substitution selected from the group consisting of C102S, C102G, C102A, C102T, C102Q, C102N, C102Y, preferably C102S.

According to the present invention, the one or more antigens to which the binding molecule of the present invention may bind can be selected form the group consisting of but not limited to: ABCF1; ACVR1; ACVR1B; ACVR2; ACVR2B; ACVRL1; ADORA2A; Aggrecan; AGR2; AICDA; AIF1; AIG1; AKAP1; AKAP2; ALK; AMH; AMHR2; ANGPT1; ANGPT2; ANGPTL3; ANGPTL4; ANPEP; APC; APOC1; AR; AXL; AZGP1(zinc-a-glycoprotein); B7.1; B7.2; BAD; BAFF; BAFF-R; BAG1; BAIl; BCL2; BCL6; BCMA; BDNF; BLNK; BLR1 (MDR15); BlyS; BMP1; BMP2; BMP3B (GDF10); BMP4; BMP6; BMP8; BMPR1A;BMPR1BBMPR2; BPAG1 (plectin); BRCA1; B7-H3; Cl9orf10(IL27w); C1s; C3; C4A; C5; C5R1; CA-125; CANT1; CASP1; CASP4; CAV1; CCBP2 (D6/JAB61); CCL1 (1-309); CCL11 (eotaxin); CCL13 (MCP-4); CCL15 (MIP-1d); CCL16 (HCC-4); CCL17 (TARC); CCL18 (PARC); CCL19 (MIP-3b); CCL2 (MCP -1); MCAF; CCL20 CMJP-3a); CCL21 (MIP-2); SLC; exodus-2; CCL22 (MDC/STC-1); CCL23 (MPIF-1); CCL24 (MPIF-2/eotaxin-2); CCL25 (TECK); CCL26 (eotaxin-3); CCL27 (CTACK/ILC); CCL28; CCL3 (MIP-1a); CCL4 (MIP-1b); CCL5 (RANTES); CCL7 (MCP-3); CCL8 (mcp-2); CCNA1; CCNA2; CCND1; CCNE1; CCNE2; CCR1 (CKR1/HM145); CCR2 (mcp-1RB/RA); CCR3 (CKR3 CMKBR3); CCR4; CCRS (CMKBR5/ChemR13); CCR6 (CMKBR6/CKR-L3 STRL22/DRY6); CCR7 (CKR7 EB11); CCR8 (CMKBR81/TER1/CKR-L1); CCR9 (GPR-9-6); CCRL1 (VSHK1); CCRL2 (L-CCR); CD164; CDS; CD7; CD15; CD19; CD1G; CD11a; CD20; CD200; CD22; CD23; CD24; CD25; CD27; CD28; CD30; CD33; CD37; CD38; CD3E; CD3G; CD3Z; CD4; CD40; CD4OL; CD41; CD4SRB; CD51; CD52; CD56; CD6; CD62L; CD70; CD72; CD73; CD74; CD79A; CD79B; CDB; CD80; CD81; CD83; CD86; CD105; CD117; CD123; CD125; CD137L; CD137; CD147; CD152; CD154; CD221; CD276; CD279; CD319; CDH1 (Ecadherin); CDH10; CDH12; CDH13; CDH18; CDH19, CDH20; CDHS; CDH7; CDH8; CDH9; CDK2; CDK3; CDK4; CDKS; CDK6; CDK7; CDK9; CDKN1A (p21Wap1/Cip1); CDKNIB (p27Kip1); CDKN1C; CDKN2A (p16INK4a); CDKN2B; CDKN2C; CDKN3; CEA; CEACAMS; CEBPB; CER1; CFD; CHGA; CHGB; Chitinase; CHST10; CKLFSF2; CKLFSF3; CKLFSF4; CKLFSFS; CKLFSF6; CKLFSF7; CKLFSF8; CLDN3; CLDN7 (claudin-7); CLDN18.2; CLN3; CLU (clusterin); cMET; CMKLR1; CMKOR1 (RDC1); CNR1; COL18A1; COL1A1; COL4A3; COL6A1; CR2; CRP; CSF1 (M-CSF); CSFR1; CSF2 (GM-CSF); CSF3 (GCSF); CTLA4; CTNNB1 (b-catenin); CTSB (cathepsin B); CX3CL1 (SCYD1); CX3CR1 (V28); CXCL1 (GRO1); CXCL10 (IP-10); CXCL11 (I-TAC/IP-9); CXCL12 (SDF1); CXCL13; CXCL14; CXCL16; CXCL2 (GRO2); CXCL3 (GRO3); CXCLS (ENA-78/LIX); CXCL6 (GCP-2); CXCL9 (MIG); CXCR3 (GPR9/CKR-L2); CXCR4; CXCR6 (TYMSTR/STRL33/Bonzo); CYBS; CYC1; CYSLTR1; DAB2IP; DES; DKFZp451J0118; DNCL1; DLL3; DPP4; DR3; DR4; DRS; DR6; E2F1; ECGF1; EDA1; EDA2; EDAR; EDA2R; EDG1; EpCAMEFNA1; EFNA3; EFNB2; EGF; EGFL7; EGFR; ELAC2; ENG; ENO1; ENO2; ENO3; EPHA3; EPHB4; EPO; ERBB2 (Her-2); EREG; ERK8; ESR1; ESR2; F3 (TF); F9 or F9a; F10 or F10a; FADD; FAP; FasL; FASN; FCER1A; FCER2; FCGR3A; FGF; FGF1 (aFGF); FGF10; FGF11; FGF12; FGF12B; FGF13; FGF14; FGF16; FGF17; FGF18; FGF19; FGF2 (bFGF); FGF20; FGF21; FGF22; FGF23; FGF3 (int-2); FGF4 (HST); FGF5; FGF6 (HST-2); FGF7 (KGF); FGF8; FGF9; FGFR1; FGFR2; FGFR3; FGFR4; FIGF (VEGFD); FIL1 (EPSILON); FIL1 (ZETA); FLJ12584; FLJ25530; FLRT1 (fibronectin); FLT1; folate receptor 1; FOS; FOSL1 (FRA-1); FY (DARC); GABRP (GABAa); GAGEB1; GAGEC1; GALNAC4S-GST; GATA3; gelatinase B; GD2; GD3; GDF5; GDF8; GFI1; GGT1; GITR; GITRL; GM-CSF; GNAS1; GNRH1; GPNMB; GPR2 (CCR10); GPR31; GPR44; GPR81 (FKSG80); GRCC10 (C10); GRP; GSN (Gelsolin); GSTP1; HAVCR2; HDAC4; HDAC5; HDAC7A; HDAC9; HER2; HER3; HER4; HGF; H1F1A; HIP1; histamine and histamine receptors; HLA-A; HLA-DRA; HM74; HMOX1; HMW-MAA Hsp-90; HVEM; TNF-RHUMCYT2A; ICAM-1; ICEBERG; ICOSL; ID2; IFN-a; IFNA1; IFNA2; IFNA4; IFNA5; IFNA6; IFNA7; IFNB 1; IFNgamma; IFNW1; IGBP1; IGF1; IGF1R; IGP1R; IGF2; IGFBP2; IGFBP3; IGFBP6; IGHE; IL-1; IL10; IL10RA; IL10RB; IL11; IL11RA; IL-12; IL12A; IL12B; IL12RB1; IL12RB2; IL13; IL13RA1; IL13RA2; IL14; IL15; IL15RA; IL16; IL17; IL17B; IL17C; IL17R; IL18; IL18BP; IL18R1; IL18RAP; IL19; IL1A; IL1B; IL1F10; IL1F5; IL1F6; IL1F7; IL1F8; TL1F9; IL1HY1; IL1R1; IL1R2; ILLRAP; IL1RAPL1; IL1RAPL2; IL1RL1; IL1RL2 IL1RN; IL2; IL24; IL20RA; IL21R; IL22; IL22R; IL22RA2; IL23; IL24; IL25; IL26; IL27; IL28A; IL28B; IL29; IL2RA; IL2RB; IL2RG; IL3; IL30; IL3RA; IL4; IL4R; ILS; IL5RA; IL6; IL6R; IL6ST (glycoprotein 130); IL7; IL7R; IL8; IL8RA; IL8RB; IL8RB; IL9; IL9R; ILK; INHA; INHBA; INSL3; INSL4; integrin αᵥβ₃; integrin β₇; IRAK1; IRAK2; TGA1; ITGA2; ITGA3; ITGA6 (a6 integrin); ITGAV; JTGB3; ITGB4 (b 4 integrin); JAG1; JAK1; JAK3; JUN; K6HF; KAI1; KDR; KIR2D; KITLG; KLFS (GC Box BP); KLF6; KLK10; KLK12; KLK13; KLK14;

KLK15; KLK3; KLK4; KLKS; KLK6; KLK9; KRT1; KRT19 (Keratin 19); KRT2A; KRTHB6 (hair-specific type II keratin); LAMAS; LEP (leptin); LEY; LIGHT; Lingo-p75; Lingo-Troy; LIV-1; LPS; LTA (TNF-b); LTB; LTB4R (GPR16); LTB4R2; LTBR; MACMARCKS; MAG or Omgp; MAP2K7 (c-Jun); MCAM; MCSP; MDK; MET; MER; MIB1; midkine; MIF; MIP-2; MKI67 (Ki-67); MMP2; MMP9; MSLN; MS4A1; MSMB; MT3(metallothionectin-III); MTSS1; MUC1(mucin); MUC2; MYC; MYD88; myostatin; NCA-2; NCK2; nectin-4; neurocan; NFKB1; NFKB2; NGFB (NGF); NGFR; NgRLingo; NOGO-A; NgR-Nogo66 (Nogo); NgR-p75; NgR-Troy; NME1 (NM23A); NOTCH-1; NOX5; NPPB; NROB1; NROB2; NR1D1; NR1D2; NR1H2; NR1H3; NR1H4; NR1I2; NR1I3; NR2C1; NR2C2; NR2E1; NR2E3; NR2F1; NR2F2; NR2F6; NR3C1; NR3C2; NR4A1; NR4A2; NR4A3; NR5A1; NR5A2; NR6A1; NRP1; NRP2; NT3; NT4; NT5E; NTN4; ODZ1; OPRD1; OX40; OX40L; P2RX7; PAP; PART1; PATE; PAWR; PCA3; PCDC1; PCNA; PCSK9; PD1; PD-L1; PDGFA; PDGFB; PDGR; igfPECAM1; PF4 (CXCL4); PGF; PGR; phosphacan; PIAS2; PIK3CG; PLAU (uPA); uPAR; PLG; PLXDC1; PPBP (CXCL7); PPID; PR1; PRKCQ; PRKD1; PRL; PROC; PROK2; PSAP; PSCA; PSMA; PTAFR; PTEN; PTGS2 (COX-2); PTN; PTK7; VEGFR1; VEGFR2; VEGFR3; RAC2 (p21Rac2); RANK; RANKL; RARB; RELT; RET; RGS1; RGS13; RGS3; RNF110 (ZNF144); ROBO2; RON; ROR1; ROR2; RYK; S100A2; SCGB1D2 (lipophilin B); SCGB2A1 (mammaglobin 2); SCGB2A2 (mammaglobin 1); SCYE1 (endothelial Monocyte-activating cytokine); SDF2; SERPINA1; SERPINA3; SERPINB5 (maspin); SERPINE1 (PAI-1); SERPINF1; SHBG; SLA2; SLC2A2; SLC33A1; SLC43A1; SLIT2; SPAK; SPP1; SPRR1B (Spr1); SOST; ST6GAL1; STAB1; STATE; STEAP; STEAP2; TACl; TAG-72; tau protein; TB4R2; TBX21; TCP10; TDGF1; TEK; TGFA; TGFB1; TGPB1I1; TGFB2; TGFB3; TGFBI; TGFBR1; TGFBR2; TGFBR3; TH1L; THBS1 (throrttbospondin-1); THBS2; THBS4; THPO; TIE (Tie-1); TIE-1; TIE-2; TIMP3; tissue factor; TLR10; TLR2; TLR3; TLR4; TLRS; TLR6; TLR7; TLR8; TLR9; TNF; TNF-a; TNF-b; TNFAIP2 (B94); TNFAIP3; TNFRSF11A; TNFRSF 1A; TNFRSF 1B; TNFRSP21; TNFRSF5; TNFRSF6 (Fas); TNFRSF7; TNFRSF8; TNFRSF9; TNFSF10 (TRAIL); TNFSF11 (TRANCE); TNFSF12 (APO3L); TNFSF13 (April); TNPSF13B; TNFSF14 (HVEM-L); TNFSF15 (VEGI); TNFSF18; TNFSF4 (OX40 ligand); TNFSF5 (CD40 ligand); TNFSF6 (FasL); TNFSF7 (CD27 ligand); TNFSF8 (CD30 ligand); TNFSF9 (4-1BB ligand); TNF-R1; TNF-R2; TOLLIP; Toll-like receptors; TOP2A (topoisomerase Iia); TP53; TPM1; TPM2; TRADD; TRAF1; TRAF2; TRAF3; TRAF4; TRAF5; TRAF6; TRAIL-R1; TRAIL-R2; TRAIL-R3; TRAIL-R4; TREM1; TREM2; TRPC6; TROY; TSLP; TWEAK; TYRO3; TYRP1; VAP-1; VEGF; VEGFB; VEGFC; versican; VHL C5; vimentin; VLA-4; VWF; XCL1 (lymphotactin); XCL2 (SCM-1b); XCR1 (GPR5/CCXCR1); YY1; and ZFPM2.

According to a preferred embodiment, the binding molecule of the invention binds to a trigger molecule on an immune effector cell, such as CD3 on T-cells as part of the T-cell receptor (TCR) (Brinkmann & Kontermann, 2017, mAbs 9:182-212). According to the present invention, the trigger molecule of the immune effector cell is selected from the group consisting of but not limited to CD2, CD3, CD16, CD44, CD64, CD69, CD89, Me114, Ly-6.2C, TCR-complex, Vy9V52 TCR, or NKG2D.

According to a further embodiment, the binding molecule of the invention binds to MET (5D5) (Jin et al., 2008, Cancer Res. 68, 4360-4368).

A most preferred embodiment of the bivalent, trivalent or tetravalent binding molecule according to the present invention binds with one, two or three binding sites to HER3, and with the respective remaining one, two or three binding sites to CD3. Example 2 provides an example of a preferred bispecific and bivalent binding molecule binding to HER3 and CD3.

A further most preferred embodiment of the bivalent, trivalent or tetravalent binding molecule according to the present invention binds with one, two or three binding sites to HER3, and with the remaining one, two or three binding sites to MET. Even more preferably, the binding molecule of the invention is bispecific and bivalent binding to MET and HER3, as exemplified in Example 1 herein below.

The binding molecule of the present invention may further comprise a peptide leader sequence; one or more molecules that aid in purification, preferably a hexahistidyl-tag or FLAG-tag; and/or one or more co-stimulatory molecules.

In embodiments in which the binding molecule of the invention is used for cell-cell recruitment, e.g. an immune effector cell like a T cell or macrophage is recruited to a tumor cell, it is preferred that two valencies of the binding molecule bind to the tumor cell, and the one or two remaining valencies binds to the immune effector cell. This approach provides high avidity binding to the tumor cell on one hand and prevents immune effector cell activation which may result from bivalent binding of a target on the immune effector cell.

According to the present invention, the modified EHD2 domain has an amino acid sequence with at least 70% amino acid identity to SEQ ID NO: 1. Preferably, the deviations from SEQ ID NO: 1 are by conservative substitutions. More preferably, these substitutions do not include substitutions of one or more amino acid residues to cysteine. The mutations introduced preferably do not give rise to formation of inter-chain disulfide bonds, i.e. additional disulfide bonds between a first modified EHD2 domain and a second modified EHD2 domain. “Conservative substitutions” may be made, for instance, on the basis of similarity in polarity, charge, size, solubility, hydrophobicity, hydrophilicity, and/or the amphipathic nature of the amino acid residues involved. Amino acids can be grouped into the following six standard amino acid groups:
(1) hydrophobic: Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

As used herein, “conservative substitutions” are defined as exchanges of an amino acid by another amino acid listed within the same group of the six standard amino acid groups shown above. For example, the exchange of Asp by Glu retains one negative charge in the so modified polypeptide. In addition, glycine and proline may be substituted for one another based on their ability to disrupt a-helices. Some preferred conservative substitutions within the above six groups are exchanges within the following sub-groups: (i) Ala, Val, Leu and Ile; (ii) Ser and Thr; (ii) Asn and Gln; (iv) Lys and Arg; and (v) Tyr and Phe. Given the known genetic code, and recombinant and synthetic DNA techniques, the skilled scientist readily can construct DNAs encoding the conservative amino acid variants.

As used herein, “non-conservative substitutions” or “non-conservative amino acid exchanges” are defined as exchanges of an amino acid by another amino acid listed in a different group of the six standard amino acid groups (1) to (6) shown above.

Particularly preferred molecules according to the invention comprise:

(1) constant part with light chain V_{L}3-43-C_{L}λ (SEQ ID NO: 13) and heavy chain V_{H}3-43-C_{H}1-Fc_{knob} (SEQ ID NO: 14), in combination with either (i) HC1-LC2 resulting in eIgG1 (heavy chain: V_{H}5D5-EHD2-1(N39Q)-Fcₕₒₗₑ (SEQ ID NO: 12), light chain: V_{L}5D5-EHD2-2 (SEQ ID NO: 11)), or (ii) HC2-LC1 resulting in eIgG2 (heavy chain: V_{H}5D5-EHD2-2-Fcₕₒₗₑ (SEQ ID NO: 10), light chain: V_{L}5D5-EHD2-1(N39Q) (SEQ ID NO: 9));

(2) light chain V_{L}3-43-C_{L}λ (SEQ ID NO: 13) and heavy chain V_{H}3-43-C_{H}1-Fc_{knob} (SEQ ID NO: 14) with eIgG (heavy chain: V_{H}huU3-EHD2-2-Fcₕₒₗₑ (SEQ ID NO: 16), light chain: V_{L}huU3-EHD2-1(N39Q) (SEQ ID NO: 15));

(3) light chain as specified in SEQ ID NO: 13 or 15, and heavy chain as specified in SEQ ID NO: 14, 16, 20, 39, 40 or 41;

(4) light chain V_{L}3-43-C_{L}λ (SEQ ID NO: 13) paired with heavy chain V_{H}3-43-C_{H}1-Fc_{knob} (SEQ ID NO: 14), and light chain V_{L}hu36-EHD2-1(N39Q) (SEQ ID NO: 43) paired with heavy chain V_{H}hu36-EHD2-2-Fcₕₒₗₑ (SEQ ID NO: 42);

(5) light chain V_{L}3-43-EHD2-1(N39Q) (SEQ ID NO: 23) and heavy chain V_{H}343-EHD2-2-Fc (SEQ ID NO: 22);

(6) light chains V_{L}3-43-EHD2-1(N39Q) (SEQ ID NO: 23) and V_{L}hu225-CLκ (SEQ ID NO: 25), and heavy chain V_{H}hu225-C_{H}1-V_{H}3-43-EHD2-2-Fc (SEQ ID NO: 24);

(7) light chains V_{L}3-43-EHD2-1(N39Q) (SEQ ID NO: 23) and V_{L}hu225-CLκ (SEQ ID NO: 25), heavy chain V_{H}hu225-C_{H}1-V_{H}3-43-EHD2-2 (SEQ ID NO: 44);

(8) a first molecule comprising light chain V_{L}hu36-CLκ (SEQ ID NO: 45) and heavy chain V_{H}hu36-C_{H}1-Fc(hole) (SEQ ID NO: 46), with CD3-targeting arm with light chain V_{H}2C11-EHD2-1(N39Q) (SEQ ID NO: 47) and heavy chain V_{L}2C11-EHD2-2-Fc(knob) (SEQ ID NO: 48), and a second molecule comprising light chain V_{L}2C11-EHD2-1(N39Q) (SEQ ID NO: 49) and heavy chain V_{H}2C11-EHD2-2-Fc(knob) (SEQ ID NO: 50);

(9) light chains V_{L}S309-CLκ (SEQ ID NO: 51) and V_{L}P2B-2F6-EHD2-1(N39Q) (SEQ ID NO: 52), and heavy chain V_{H}S309-C_{H}1-V_{H}P2B-2F6-EHD2-2-Fc (SEQ ID NO: 53).

Most preferred molecules according to the invention comprise:

(i) SEQ ID NO: 13, 14, 15 and 16;

(ii) SEQ ID NO: 13, 15, 16 and 20;

(iii) SEQ ID NO: 13, 15, 39 and 40;

(iv) SEQ ID NO: 13, 14, 15 and 41;

(v) SEQ ID NO: 23, 24 and 25.

Further preferred molecules according to the invention comprise the molecules identified above as (1) to (9) and (i) to (v) having at least 90% sequence identity to the sequences identified above under (1) to (9) and (i) to (v), preferably 91%, 92%, 93%, 94%, 95%, 96%, 97%, 98% or 99% sequence identity to the sequences identified above under (1) to (9) and (i) to (v). It is to be understood that this sequence identity may refer to one, two, three or four of the individual sequences identified respectively under each item (1) to (9) and (i) to (v) above. Preferably, these molecules with a sequence identity of at least 90% or more to the sequences identified above under (1) to (9) and (i) to (v) further maintain essentially the same or maintain the same biological function as the respective molecule from which it is derived comprising the sequences identified above under (1) to (9) and (i) to (v). Preferably, the term “biological function” as used herein refers to binding specificity and/or affinity. Maintaining essentially the same biological function means a binding specificity and/or affinity of at least 50% of that of the respective molecule comprising the sequences identified above under (1) to (9) and (i) to (v) from which it is derived, preferably a binding specificity and/or affinity of at least 60%, 70%, 80%, 85%, 90%, 91%, 92%, 93%, 94%, 95%, 96%, 97%, 98%, 99%. Determining binding and/or affinity is well known to the skilled person and can be performed e.g. by surface plasmon resonance measurements and/or by in vitro release assays.

It will be understood by the skilled person that the molecules according to the invention may or may not carry a histidine-tag (His-tag, 6xHis tag etc.) or a similar addition or tag as exemplarily described herein at one or more of the polypeptides in order to facilitate easy purification thereof. The skilled person readily understands that such additional structure does not have a specific influence on the molecule's functional characteristics as described herein.

According to a further aspect, the present invention provides a nucleic acid or set of nucleic acids encoding the binding molecule according to the invention. Nucleic acids may be degraded by endonucleases or exonucleases, in particular by DNases and RNases which can be found in the cell. It may, therefore, be advantageous to modify the nucleic acids of the invention in order to stabilize them against degradation, thereby ensuring that a high concentration of the nucleic acid is maintained in the cell over a long period of time. Typically, such stabilization can be obtained by introducing one or more internucleotide phosphorus groups or by introducing one or more non-phosphorus internucleotides. Accordingly, nucleic acids can be composed of non-naturally occurring nucleotides and/or modifications to naturally occurring nucleotides, and/or changes to the backbone of the molecule. Modified internucleotide phosphate radicals and/or non-phosphorus bridges in a nucleic acid include but are not limited to methyl phosphonate, phosphorothioate, phosphoramidate, phosphorodithioate and/or phosphate esters, whereas non-phosphorus internucleotide analogues include but are not limited to, siloxane bridges, carbonate bridges, carboxymethyl esters, acetamidate bridges and/or thioether bridges. Further examples of nucleotide modifications include but are not limited to: phosphorylation of 5′ or 3′ nucleotides to allow for ligation or prevention of exonuclease degradation/polymerase extension, respectively; amino, thiol, alkyne, or biotinyl modifications for covalent and near covalent attachments; fluorphores and quenchers; and modified bases such as deoxyInosine (dI), 5-Bromo-deoxyuridine (5-Bromo-dU), deoxyUridine, 2-Aminopurine, 2,6-Diaminopurine, inverted dT, inverted Dideoxy-T, dideoxyCytidine (ddC 5-Methyl deoxyCytidine (5-Methyl dC), locked nucleic acids (LNA's), 5-Nitroindole, Iso-dC and —dG bases, 2′-O-Methyl RNA bases, Hydroxmethyl dC, 5-hydroxybutynl-2′-deoxyuridine, 8-aza-7-deazaguanosineand Fluorine Modified Bases. Thus, the nucleic acid can also be an artificial nucleic acid which includes but is not limited to polyamide or peptide nucleic acid (PNA), morpholino and locked nucleic acid (LNA), as well as glycol nucleic acid (GNA) and threose nucleic acid (TNA).

It should be understood that in any of above outlined basic arrangements and embodiments of the binding molecule according to the present invention, there are free N-terminal and/or C-terminal ends of the polypeptide chains which are available for the attachment of further functional groups. Alternatively, or additionally functional groups, in particular pharmaceutical active moieties and/or an imaging molecules may be coupled to side chains of amino acids within the polypeptide chains such as Lys, Arg, Glu or Asp.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL1662, hereinafter referred to as “YB2/0 cell”), HEK293 cells, and the like. Preferred host cells are HEK293-6E cells (Durocher et al., 2002, Nucl. Acids Res. 30(2)e9) or CHO cells.

Hence, according to a further aspect, the present invention provides a vector comprising the nucleic acid or set of nucleic acids of the invention. Suitable vectors for expressing nucleic acids in host cells are well known in the art.

The present invention also relates to a method of producing a recombinant host cell expressing a binding molecule according to the invention, said method comprising the steps consisting of: (i) introducing in vitro or ex vivo the nucleic acid or set of nucleic acids or the vector as described above into a competent host cell, (ii) culturing in vitro or ex vivo the recombinant host cell obtained and (iii), optionally, selecting the cells which express and/or secrete said binding molecule.

Thus, the present invention also provides a host cell comprising the vector according to the present invention.

According to a further aspect, the present invention provides a pharmaceutical composition comprising the binding molecule, the nucleic acid or set of nucleic acids, the vector, or the host cell according to the present invention as active agent, and a pharmaceutically acceptable carrier and/or suitable excipients. The pharmaceutical composition may further comprise one or more further active agents. The pharmaceutical composition is preferably selected from the group consisting of solid, liquid, semi-solid or transdermal therapeutic systems. It is envisioned that the pharmaceutical compositions of the invention comprise one or more complexes of the first aspect of the invention.

In a further aspect, the present invention relates to the binding molecule, the nucleic acid or set of nucleic acids, the vector, the host cell, or the pharmaceutical composition of the present invention for use in medicine.

The binding molecule, the nucleic acid or set of nucleic acids, the vector, the host cell, or the pharmaceutical composition of the present invention is particularly suitable for use in treating cancer.

The present invention particularly pertains to the following items:

Item 1. A binding molecule comprising:

a first polypeptide chain comprising a first binding domain (BD1) and a first modified EHD2 domain (EHD2-1), and

a second polypeptide chain comprising a second binding domain (BD2) and a second modified EHD2 domain (EHD2-2),

wherein the amino acid sequences of EHD2-1 and EHD2-2 are different from each other and each is selected from (i) an amino acid sequence with at least 70% amino acid identity to SEQ ID NO: 1 and which does not have a Cys at position 14, or (ii) an amino acid sequence with at least 70% amino acid identity to SEQ ID NO: 1 and which does not have a Cys at position 102,

wherein BD1 and BD2 together form an antigen binding site, and wherein EHD2-1 and EHD2-2 are covalently bound to each other.

Item 2. The binding molecule according to item 1, wherein one or both of the modified EHD2 domains further comprises a single amino acid substitution at position N39.
Item 3. The binding molecule according to any one of items 1 to 2, wherein BD1 and BD2 are different from each other and each is selected from a V_{H} and a V_{L} or from a variable region of a TCR α-chain and a variable region of a TCR β-chain.
Item 4. The binding molecule according to any one of items 1 to 3, further comprising a first Fc chain.
Item 5. The binding molecule according to any one of items 1 to 4, further comprising a third binding domain (BD3) and a fourth binding domain (BD4), wherein BD3 and BD4 together form an antigen binding site.
Item 6. The binding molecule according to item 5, wherein BD3 and BD4 are different from each other and each is selected from a V_{H} and a V_{L}, or from a variable region of a TCR α-chain and a variable region of a TCR β-chain.
Item 7. The binding molecule according to items 5 or 6, wherein the binding molecule further comprises a third polypeptide chain, preferably wherein the binding molecule further comprises a third and a fourth polypeptide chain.
Item 8. The binding molecule according to any one of items 5 to 7, further comprising a second Fc chain.
Item 9. The binding molecule according to items 8, wherein the first and the second Fc chains are different from each other and form a heterodimeric Fc.
Item 10. The binding molecule according to any one of items 5 to 9, being monospecific or bispecific.
Item 11. The binding molecule according to any one of items 5 to 10, wherein the third polypeptide chain comprising BD3 further comprises one of

(i) a C_{H}1 domain,

(ii) a C_{L} domain,

(iii) a first modified EHD2 domain (EHD2-1), and

(iv) a second modified EHD2 domain (EHD2-2),

and wherein the fourth polypeptide chain comprising BD4 further comprises

in case of (i) a C_{L} domain,

in case of (ii) a C_{H}1 domain,

in case of (iii) a second modified EHD2 domain (EHD2-2), and

in case of (iv) a first modified EHD2 domain (EHD2-1),

wherein the amino acid sequences of EHD2-1 and EHD2-2 are different from each other and each is selected from an amino acid sequence with at least 70% amino acid identity to SEQ ID NO: 1 and which does not have a Cys at position 14, or an amino acid sequence with at least 70% amino acid identity to SEQ ID NO: 1 and which does not have a Cys at position 102.

Item 12. The binding molecule according to item 11, wherein one or both of the modified EHD2 domains of the third or the fourth polypeptide chain further comprises a single amino acid substitution at position N39.
Item 13. The binding molecule according to any one of items 5 to 12, further comprising a fifth binding domain (BD5) and a sixth binding domain (BD6), wherein BD5 and BD6 together form an antigen binding site.
Item 14. The binding molecule according to item 13, further comprising

(i) a C_{H}1 domain,

(ii) a C_{L} domain,

(iii) a first modified EHD2 domain (EHD2-1), or

(iv) a second modified EHD2 domain (EHD2-2)

connected to BD5, and

in case of (i) a C_{L} domain,

in case of (ii) a C_{H}1 domain,

in case of (iii) a second modified EHD2 domain (EHD2-2), and

in case of (iv) a first modified EHD2 domain (EHD2-1) connected to BD6, wherein the amino acid sequences of EHD2-1 and EHD2-2 are different from each other and each is selected from an amino acid sequence with at least 70% amino acid identity to SEQ ID NO: 1 and which does not have a Cys at position 14, or an amino acid sequence with at least 70% amino acid identity to SEQ ID NO: 1 and which does not have a Cys at position 102.

Item 15. The binding molecule according to item 14, wherein one or both of the modified EHD2 domains connected to BD5 or BD6 further comprises a single amino acid substitution at position N39.
Item 16. The binding molecule according to item 14 or 15, wherein BD5 and BD6 are different from each other and each is selected from a V_{H} and a V_{L}, or from a variable region of a TCR α-chain and a variable region of a TCR β-chain.
Item 17. The binding molecule of any one of items 14 to 16, wherein the C_{H}1 domain, the C_{L} domain, the EHD2-1 or the EHD2-2 connected to BD5 or BD6 is connected with one of BD1, BD2, BD3 or BD4 via a linker.
Item 18. The binding molecule according to any one of items 11 to 17, being monospecific, bispecific, or trispecific.
Item 19. The binding molecule according to any one of items 11 to 18, further comprising a seventh binding domain (BD7) and an eighth binding domain (BD8), wherein BD7 and BD8 together form an antigen binding site.
Item 20. The binding molecule according to item 19, further comprising

(i) a C_{H}1 domain,

(ii) a C_{L} domain,

(iii) a first modified EHD2 domain (EHD2-1), or

(iv) a second modified EHD2 domain (EHD2-2)

connected to BD7, and

in case of (i) a C_{L} domain,

in case of (ii) a C_{H}1 domain,

in case of (iii) a second modified EHD2 domain (EHD2-2), and

in case of (iv) a first modified EHD2 domain (EHD2-1) connected to BD8, wherein the amino acid sequences of EHD2-1 and EHD2-2 are different from each other and each is selected from an amino acid sequence with at least 70% amino acid identity to SEQ ID NO: 1 and which does not have a Cys at position 14, or an amino acid sequence with at least 70% amino acid identity to SEQ ID NO: 1 and which does not have a Cys at position 102.

Item 21. The binding molecule according to item 20, wherein one or both of the modified EHD2 domains connected to BD7 or BD8 further comprises a single amino acid substitution at position N39.
Item 22. The binding molecule according to item 21, wherein BD7 and BD8 are different from each other and each is selected from a V_{H} and a V_{L}, or from a variable region of a TCR α-chain and a variable region of a TCR β-chain.
Item 23. The binding molecule of any one of items 20 to 22, wherein the C_{H}1 domain, the C_{L} domain, the EHD2-1 or the EHD2-2 connected to BD7 or BD8 is connected with one of BD1, BD2, BD3 or BD4 not connected with BD5 or BD6 via a linker.
Item 24. The binding molecule according to any one of items 19 to 23, being monospecific, bispecific, trispecific or tetraspecific.
Item 25. The binding molecule according to any one of the preceding items, wherein none, one or more of the modified EHD2 domains carries one N-glycan.
Item 26. The binding molecule according to any of the preceding items, wherein the Cys at position 14 of SEQ ID NO: 1 is substituted by an amino acid selected from the group consisting of Ser, Gly, Ala, Thr, Gln, Asn, and Tyr, preferably Ser.
Item 27. The binding molecule according to any of the preceding items, wherein the Cys at position 102 of SEQ ID NO: 1 is substituted by an amino acid selected from the group consisting of Ser, Gly, Ala, Thr, Gln, Asn, and Tyr, preferably Ser.
Item 28. The binding molecule according to any of the preceding items, wherein the single amino acid substitution at position N39 is N39Q.
Item 29. A nucleic acid or set of nucleic acids encoding the binding molecule according to any of items 1 to 28.
Item 30. A vector comprising the nucleic acid or set of nucleic acids of item 29.
Item 31. A host cell comprising the vector according to item 30.
Item 32. A pharmaceutical composition comprising the binding molecule according to any of items 1 to 28, the nucleic acid or set of nucleic acids according to item 29, the vector according to item 30, or the host cell according to item 31, and a pharmaceutically acceptable carrier.
Item 33. A binding molecule comprising SEQ ID NO: 13, SEQ ID NO: 14, SEQ ID NO: 15 and SEQ ID NO: 16.
Item 34. A binding molecule comprising SEQ ID NO: 13, SEQ ID NO: 15, SEQ ID NO: 16 and SEQ ID NO: 20.
Item 35. A binding molecule comprising SEQ ID NO: 13, SEQ ID NO: 15, SEQ ID NO: 39 and SEQ ID NO: 40.
Item 36. A binding molecule comprising SEQ ID NO: 13, SEQ ID NO: 14, SEQ ID NO: 15 and SEQ ID NO: 41.
Item 37. A binding molecule comprising SEQ ID NO: 23, SEQ ID NO: 24 and SEQ ID NO: 25.
Item 38. A nucleic acid or set of nucleic acids encoding the binding molecule according to any of items 33 to 37.
Item 39. A vector comprising the nucleic acid or set of nucleic acids of item 38.
Item 40. A host cell comprising the vector according to item 39.
Item 41. A pharmaceutical composition comprising the binding molecule according to any of items 33 to 37, the nucleic acid or set of nucleic acids according to item 38, the vector according to item 39, or the host cell according to item 40, and a pharmaceutically acceptable carrier.
Item 42. The binding molecule according to any of items 33 to 37, the nucleic acid or set of nucleic acids according to item 38, the vector according to item 39, the host cell according to item 40, or the pharmaceutical composition according to item 41 for use in medicine, preferably for use in the treatment of cancer.
Item 43. A method of treatment, comprising administering to a patient in need thereof a therapeutically effective amount of the binding molecule according to any of items 33 to 37, the nucleic acid or set of nucleic acids according to item 38, the vector according to item 39, the host cell according to item 40, or the pharmaceutical composition according to item 41.
Item 44. A method of treating cancer, comprising administering to a patient in need thereof a therapeutically effective amount of the binding molecule according to any of items 33 to 37, the nucleic acid or set of nucleic acids according to item 38, the vector according to item 39, the host cell according to item 40, or the pharmaceutical composition according to item 41.
Item 45. The binding molecule according to any of items 1 to 28, the nucleic acid or set of nucleic acids according to item 29, the vector according to item 30, the host cell according to item 31, or the pharmaceutical composition according to item 32 for use in medicine, preferably for use in the treatment of cancer.
Item 46. A method of treatment, comprising administering to a patient in need thereof a therapeutically effective amount of the binding molecule according to any of items 1 to 28, the nucleic acid or set of nucleic acids according to item 29, the vector according to item 30, the host cell according to item 31, or the pharmaceutical composition according to item 32.
Item 44. A method of treating cancer, comprising administering to a patient in need thereof a therapeutically effective amount of the binding molecule according to any of items 1 to 28, the nucleic acid or set of nucleic acids according to item 29, the vector according to item 30, the host cell according to item 31, or the pharmaceutical composition according to item 32.

### Examples

### Example 1: Bispecific Bivalent anti-cMET x anti-HER3 eIgGs

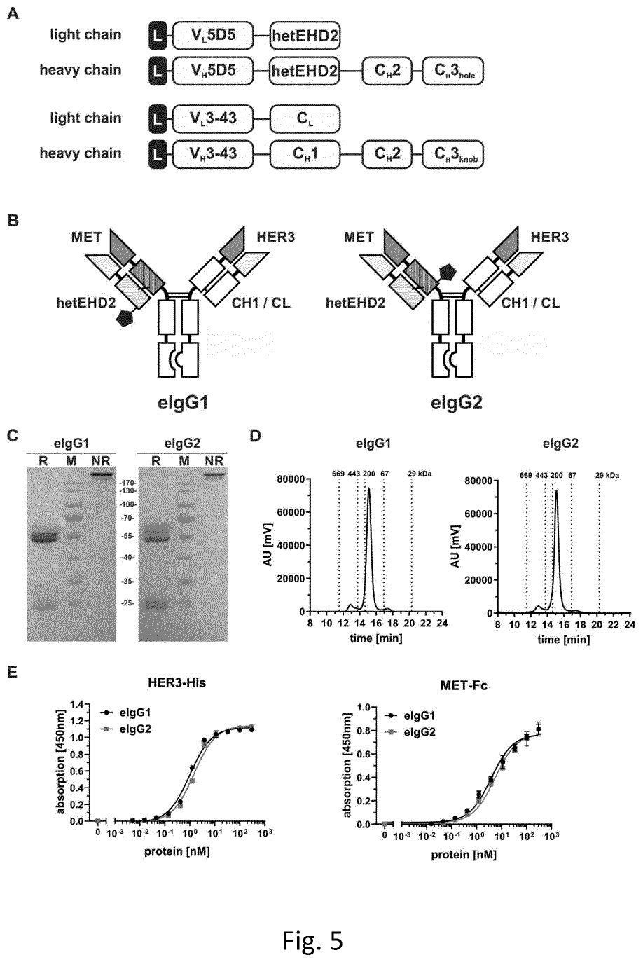

Bispecific bivalent eIgG molecules were generated by combining a Fv domain, specific for HER3 (3-43) (Schmitt et al., 2017, mAbs 9, 831-843), with the C_{H}1/C_{L} heterodimer of IgG1, a Fv domain, specific for MET (5D5) (Jin et al., 2008, Cancer Res. 68, 4360-4368), with the modified C_{H}2 domain of IgE (hetEHD2), and a heterodimeric Fc part (knob-into-hole technology). Thus, the bispecific molecules consist of four different polypeptide chains. The constant part of the different antibodies consists of the light chain V_{L}3-43-C_{L}λ (SEQ ID NO: 13) and the heavy chain V_{H}3-43-C_{H}1-Fc_{knob} (SEQ ID NO: 14). The second part consist of the EHD2 configuration either in HC1-LC2 resulting in eIgG1 (heavy chain: V_{H}5D5-EHD2-1(N39Q)-Fcₕₒₗₑ (SEQ ID NO: 12); light chain: V_{H}5D5-EHD2-2 (SEQ ID NO: 11)), or in HC2-LC1 resulting in eIgG2 (heavy chain: V_{H}5D5-EHD2-2-Fcₕₒₗₑ (SEQ ID NO: 10); light chain: V_{L}5D5-EHD2-1(N39Q) (SEQ ID NO: 9)). The bispecific, bivalent eIgG molecules exhibit one antigen binding site for HER3 and one binding site for MET. The respective polypeptide chains are exemplarily and schematically shown in FIG. 5A, the resulting binding molecules are schematically shown in FIG. 5B.

The bispecific, bivalent eIgG molecules were expressed in transiently transfected HEK293-6E cells after co-administration of the four plasmids encoding for both heavy chains and both light chains, using polyethylenimine as transfection reagent. Protein secreted into cell culture supernatant was purified using Protein A affinity chromatography. SDS-PAGE analysis revealed 4 major bands under reducing conditions: two bands at approximately 55 kDa corresponding most likely to the two heavy chains, and two bands at approximately 25 kDa corresponding most likely to the two light chains of the antibody. In addition, one slight band at approximately 30 kDa was also observed for eIgG1corresponding most likely to the glycosylated version of the light chain containing EHD2-2 (V_{L}5D5-EHD2-2), and one slight band at approximately 65 kDa for eIgG2 corresponding most likely to the glycosylated form of the heavy chain containing EHD2-2 (V_{H}5D5-EHD2-2-Fcₕₒₗₑ). Under non-reducing conditions, one major band at approximately 200 kDa was observed corresponding most likely to the intact antibody composed of the four different polypeptide chains (FIG. 5C). Purity, integrity and homogeneity of both bispecific, bivalent eIgG molecules were confirmed by size exclusion chromatography (FIG. 5D). Binding of both eIgG molecules to the extracellular domain (ECD) of HER3 (aa 21-643) and MET (aa 25-787) was determined by ELISA. The His-tagged HER3 protein and the MET Fc fusion protein were coated onto polystyrene microtiter plates at a concentration of 2 μg/ml diluted in PBS. Remaining binding sites were blocked with PBS, 2% skimmed milk (MPBS). Plates were then incubated with serial dilution of the bispecific eIgG antibodies. After washing, bound antibodies were detected with an HRP-conjugated anti-human Fc antibody using HER3-His antigen and an HRP-conjugated anti-human Fab antibody using MET-Fc antigen and TMB, H₂O₂ as substrate. Both bispecific, bivalent eIgG antibodies showed concentration-dependent binding to HER3-His and MET-Fc with EC₅₀ values in the nanomolar range (1.0 and 1.4 nM for HER3; 3.8 and 5.4 nM for MET, for eIgG1 and eIgG2, respectively) (FIG. 5E). These experiments confirmed binding of both bispecific, bivalent eIgGs antibodies to both antigens, HER3 and MET, in the expected range (Schmitt et al., 2017, mAbs 9, 831-843).

### Example 2: A Bispecific and Bivalent Anti-CD3x Anti-HER3 eIgG

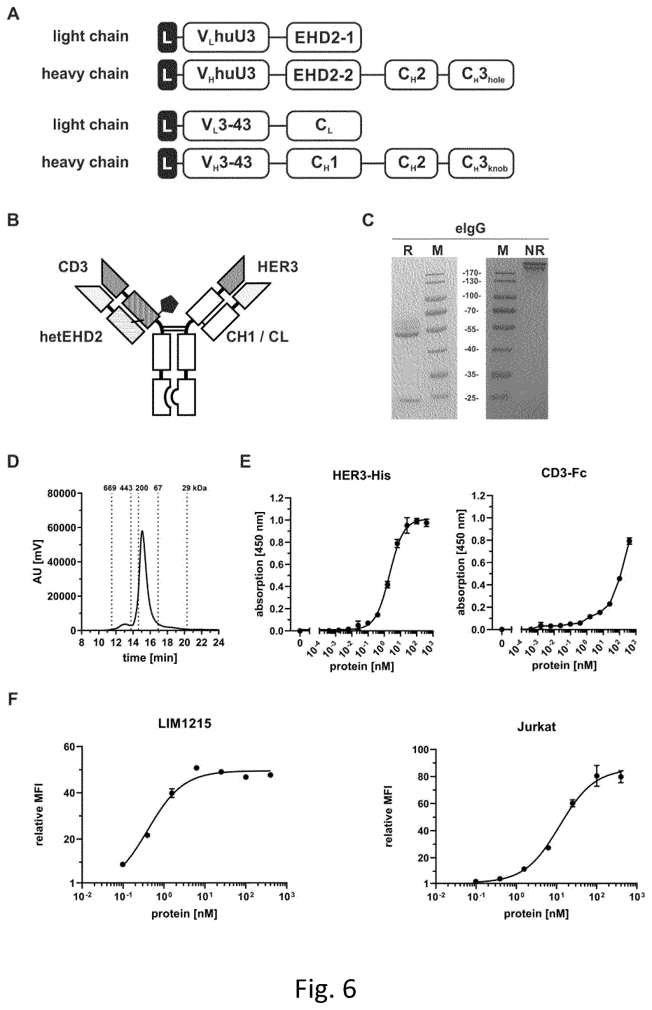

Bispecific bivalent eIgG molecules were generated by combining a Fv domain, specific for HER3 (3-43) (Schmitt et al., 2017, mAbs 9, 831-843), with the CH1/C_{L} heterodimer of IgG1, a Fv domain, specific for CD3 (huU3), with the modified C_{H}2 domain of IgE (hetEHD2), and a heterodimeric Fc part (knob-into-hole technology). The CD3 binding site consists of a humanized version of the anti-CD3 mAb UCHT1. Thus, the bispecific molecules consist of four different polypeptide chains. The first part of the different antibodies consists of the light chain V_{L}3-43-C_{L}λ (SEQ ID NO: 13) and the heavy chain V_{H}3-43-C_{H}1-Fc_{knob} (SEQ ID NO: 14). The second part consists of the EHD2 configuration in HC2-LC1 resulting in eIgG (heavy chain: V_{H}huU3-EHD2-2-Fcₕₒₗₑ (SEQ ID NO: 16); light chain: V_{L}huU3-EHD2-1(N39Q) (SEQ ID NO: 15)). The bispecific, bivalent eIgG molecules exhibit one antigen binding site for HER3 and one binding site for CD3. The respective polypeptide chains are exemplarily and schematically shown in FIG. 6A, the resulting binding molecule is schematically shown in FIG. 6B.

The bispecific, bivalent eIgG molecule was expressed in transiently transfected HEK293-6E cells after co-administration of the four plasmids encoding for both heavy chains and both light chains, using polyethylenimine as transfection reagent. Protein secreted into cell culture supernatant was purified using Protein A affinity chromatography. SDS-PAGE analysis revealed 2 major bands under reducing conditions: one band at approximately 50 kDa corresponding to the V_{H}3-43-C_{H}1-Fcknob heavy chains, and one band at approximately 25 kDa corresponding to the two light chains of the antibody. In addition, two slight bands were observed at approximately 60 and 65 kDa corresponding to (the non-glycosylated and glycosylated form of) the heavy chain containing EHD2-2 (V_{H}huU3-EHD2-2-Fcₕₒₗₑ). Under non-reducing conditions, one major band at approximately 200 kDa was observed corresponding to the intact antibody composed of the four different polypeptide chains (FIG. 6C). Purity, integrity and homogeneity of the bispecific, bivalent eIgG molecule were confirmed by size exclusion chromatography (FIG. 6D). Binding of the eIgG molecule to the extracellular domain (ECD) of HER3 (aa 21-643) and CD3 was determined by ELISA. The His-tagged HER3 protein and the CD3-Fc fusion protein were coated onto polystyrene microtiter plates at a concentration of 2 μg/ml diluted in PBS. Remaining binding sites were blocked with PBS, 2% skimmed milk (MPBS). Plates were then incubated with serial dilution of the bispecific eIgG antibody. After washing, bound antibodies were detected with an HRP-conjugated anti-human Fc antibody using HER3-His antigen and an HRP-conjugated anti-human Fab antibody using CD3-Fc antigen and TMB, H₂O₂ as substrate. The bispecific, bivalent eIgG antibody showed concentration-dependent binding to HER3-His and CD3-Fc with EC₅₀ values of 2.1 nM for HER3 (FIG. 6E). These experiments confirmed binding of the bispecific, bivalent eIgG antibody to both antigens, HER3 and CD3. Binding studies of bispecific eIgG antibody to HER3-expressing (LIM1215) cells and CD3-expressing (Jurkat) cells was analyzed via flow cytometry. Adherent LIM1215 cells were washed with PBS and shortly trypsinized at 37° C. Trypsin was quenched with FCS containing medium and removed by centrifugation (500 xg, 5 minutes). The suspension Jurkat cells were used without the usage of trypsin. 100,000 cells per well were seeded and incubated with a serial dilution of the bispecific eIgG antibody diluted in PBA (PBS containing 2% (v/v) FCS, 0.02% (w/v) NaN₃) for one hour at 4° C. Cells were washed twice using PBA. Bound antibodies were detected using PE-labeled anti-human Fc secondary antibody, which was incubated for another hour at 4° C. After washing, median fluorescence intensity (MFI) was measured with a Milltenyi MACSQuant® Analyzer 10. Relative MFI (to unstained cells) were calculated by MACSQuant® software and Excel. The eIgG antibody bound to the cells in a concentration-dependent manner with EC₅₀ values of 0.4 nM using LIM1215 and 11.9 nM using Jurkat cells (FIG. 6F).

### Example 3: Protein Engineering for the Generation of Heterodimeric hetEHD2 Domains

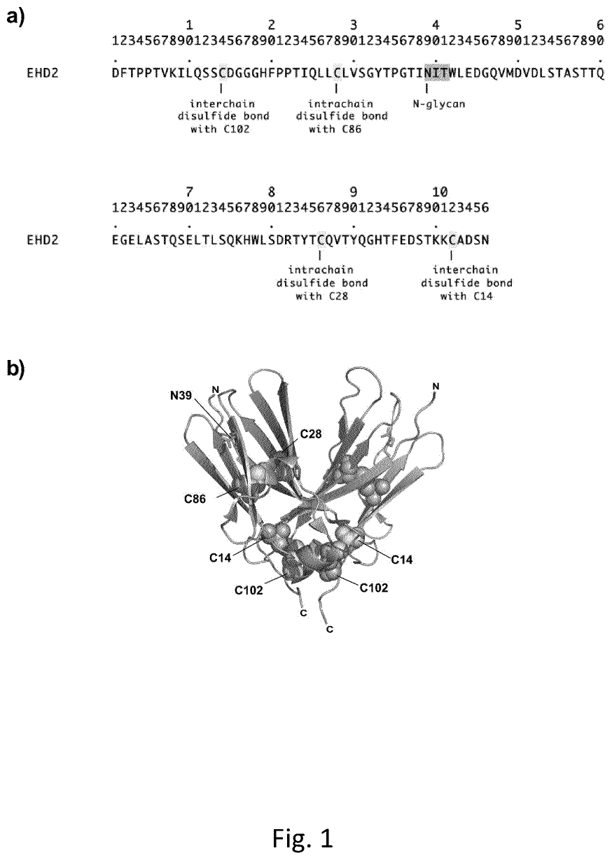

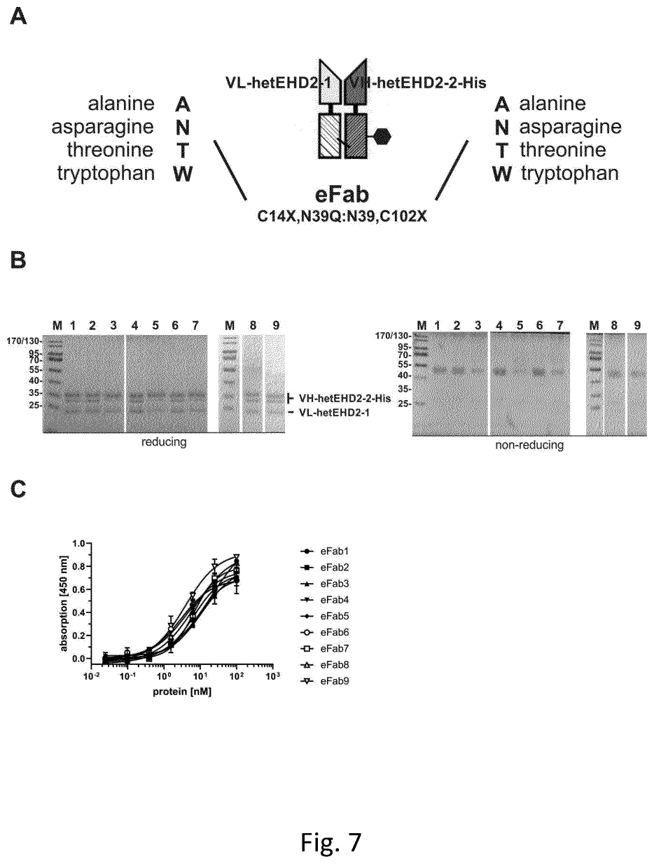

The position C14 on chain A and the position C102 on chain B were substituted with different residues (A, T, N, S, W) to evaluate their effect on the formation of functional heterodimeric eFab molecules (hetE-Fab). As a model, a hetE-Fab was used consisting of the variable domains of the IgG 3-43 (anti-HER3 targeting) fused to the two hetEHD2 domains (FIG. 1A). Molecules were generated by site directed mutagenesis using the Q5® Site-Directed Mutagenesis Kit (NEB). After confirming the correct substitution of the different residues, HEK293-6E cells were co-transfected with different combinations of two plasmids (FIG. 7A and Table 1) and proteins were purified from cell culture supernatant by affinity chromatography using Ni-NTA resins, as all molecules contained the His-tag on the VH-hetEHD2 chain. Concentration of the molecules was determined photometrically at 280 nM using the NanoDrop ND-1000 and yields were calculated for the used volume of supernatant.

TABLE 1
List of different Fv3-43-hetEHD2 (eFab) molecules with different residues in the
position C14 of chain A and C102 in chain B. Molecules bearing different combinations
were produced, analyzed concerning yield and binding to HER3 (EC₅₀ values)
used as immobilized antigen in ELISA. SEQ IDs NO are listed in the table.

| eFab | Fd (V_{H}3-43-hetEHD2-2-His) | | | | light chain (V_{L}3-43-hetEHD2-1) | | | | yield | HER3 binding |
|---|---|---|---|---|---|---|---|---|---|---|
| molecule | C14 | N39 | C102 | SEQ ID NO | C14 | N39 | C102 | SEQ ID NO | [mg/L] | [nM] |
| 1 | C | N | A | 27 | A | Q | C | 32 | 1.6 | 12.1 |
| 2 | C | N | W | 28 | T | Q | C | 33 | 9.5 | 7.3 |
| 3 | C | N | N | 29 | A | Q | C | 32 | 8.3 | 4.8 |
| 4 | C | N | T | 30 | N | Q | C | 34 | 12.1 | 3.0 |
| 5 | C | N | A | 27 | W | Q | C | 35 | 0.9 | 11.0 |
| 6 | C | N | N | 29 | T | Q | C | 33 | 12.7 | 4.1 |
| 7 | C | N | T | 30 | W | Q | C | 35 | 12.3 | 7.1 |
| 8 | C | N | T | 30 | T | Q | C | 33 | 6.8 | 6.2 |
| 9 | C | N | S | 31 | S | Q | C | 36 | 1.6 | 3.7 |

All eFab combinations could be produced, although with varying yields (Table 1). Proteins were further analyzed by SDS-PAGE under reducing and non-reducing conditions (FIG. 7B). Reducing conditions confirmed presence of two different chains with the expected size of approximately 27 kDa for VH-hetEHD2-2 fragment (carrying an N-glycan in the hetEHD2 domain) and 23 kDa for the non-glycosylated light chain. Non-reducing conditions confirmed dimeric assembly and disulfide linkage of the molecules. Finally, the different eFab molecules were used to determine binding to the immobilized extracellular domain (ECD) of HER3 (aa 21-643) fused to an Fc part in ELISA (FIG. 7C). The HER3-Fc fusion protein was immobilized onto polystyrene microtiter plates at a concentration of 2 μg/ml diluted in PBS. Residual binding sites were blocked with PBS, 2% skimmed milk (MPBS). Plates were then incubated with serial dilutions of the different eFab molecules. After washing, bound eFab molecules were detected with an HRP-conjugated anti-His antibody. TMB and H₂O₂ were used as substrate. All eFab molecules showed concentration-dependent binding to HER3-Fc fusion protein with EC₅₀ values in the nanomolar range (Table 1).

In summary, the data show that functional eFab molecules can be generated by introducing different combinations of substitutions into the hetEHD2 domains (position Cys14 in chain A and the position Cys102 in chain B), e.g. using alanine, asparagine, threonine, or tryptophan, thereby removing one of the original covalent disulfide bonds between C14 and C102. The finding that different combinations of residues can be introduced without affecting heterodimer formation and antigen binding indicates an unexpected degree of plasticity at these positions. Interestingly, some of the substitution, e.g. asparagine (N) in one chain and alanine (A) or threonine (T) in the other chain, resulted in eFabs which were slightly better than others regarding productivity and antigen-binding.

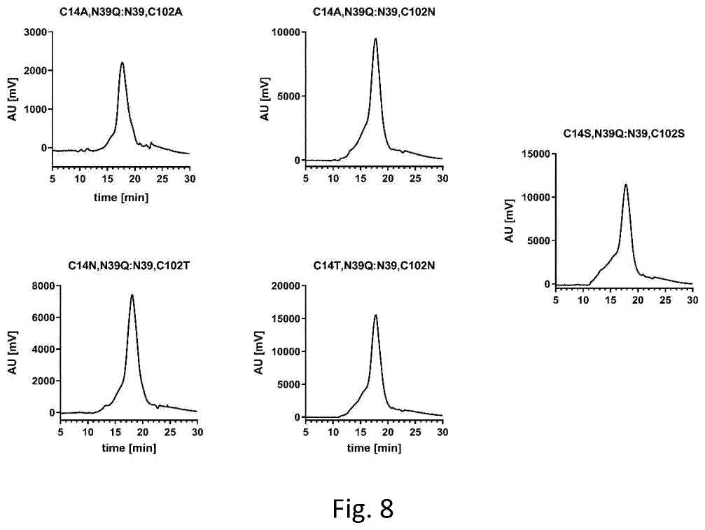

In a second experiment, four of the new eFab molecules (C14A,N39Q:N39,C102A; C14A,N39Q:N39,C102N; C14N,N39Q:N39,C102T; C14T,N39Q:N39,C102N) and the originally used eFab (C14S,N39Q:N39,C102S) were analyzed. Again, molecules were produced in co-transfected HEK293-6E suspension cells and purified using Ni-NTA affinity purification. Integrity of molecules were analyzed by size-exclusion chromatography. Here, a single peak of the correct size was observed for all molecules, demonstrating correct formation of heterodimers for all variants (FIG. 8).

### Example 4: Different hetEHD2 Molecules with Different Glycosylation

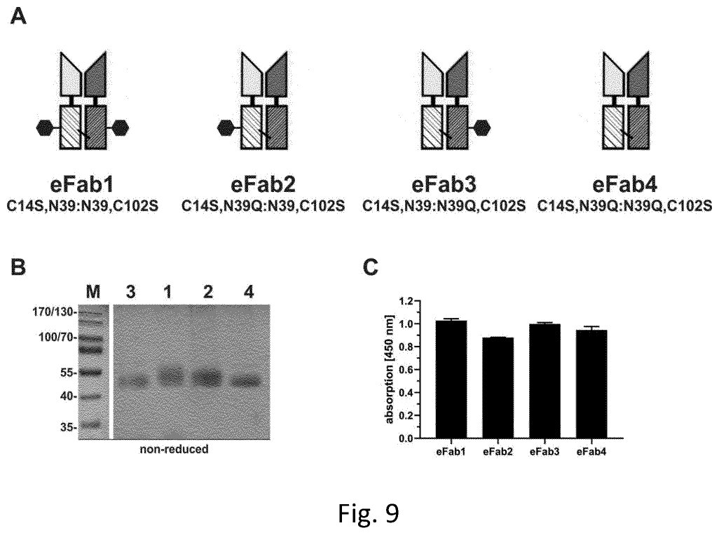

Based on the novel design for the formation of EHD2 heterodimers, the serine substitution on both chains (chain A: C14S; chain B: C102S) was used to study the effects of varying N-glycosylation of the hetEHD2 domains on expression and formation of functional heterodimers. Naturally, the EHD2 domains carry an N-glycan at position N39. Using the eFab targeting HER3, an eFab (eFab l) with N-glycans on both EHD2 domains (C14S,N39 (SEQ ID NO: 37):N39,C102S (SEQ ID NO: 31)), two eFabs with only one glycosylated EHD2 moiety (eFab2: C14S,N39Q (SEQ ID NO: 36):N39,C102S (SEQ ID NO: 31); eFab3: C14S,N39 (SEQ ID NO: 37):N39Q,C102S (SEQ ID NO: 38)), and one molecule (eFab4) without any N-glycosylation in EHD2 (C14S,N39Q (SEQ ID NO: 36):N39Q,C102S (SEQ ID NO: 38)) were generated. The resulting molecules are schematically shown in FIG. 9A. The V_{H}-hetEHD2 chains further contained a His-tag for purification and detection. The monovalent eFab molecules were expressed in HEK293-6E cells after transient co-transfection of the two plasmids encoding for V_{H}-hetEHD2-2 chain and V_{L}-hetEHD2-1 chain using polyethylenimine as transfection reagent. Proteins secreted into cell culture supernatant were purified using Ni-NTA affinity chromatography. Under non-reducing conditions in SDS-PAGE analysis, one major band at approximately 50 kDa was observed corresponding to the intact eFab molecule composed of the two different polypeptide chains (FIG. 9B).

Binding of the different Fab molecules to the extracellular domain (ECD) of HER3 (aa 21-643) was determined by ELISA (FIG. 9C). The HER3-Fc fusion protein was coated onto polystyrene microtiter plates at a concentration of 2 μg/ml diluted in PBS. Residual binding sites were blocked with PBS, 2% skimmed milk (MPBS). Plates were then incubated with 100 nM of the different Fab molecules. After washing, bound molecules were detected with an HRP-conjugated anti-His antibody using HER3-Fc as immobilized antigen. TMB and H₂O₂ were used as substrate. All four different Fab molecules showed similar binding to HER3-Fc (FIG. 9C). These experiments confirmed the formation of functional heterodimers using different glycosylated derivatives of the hetEHD2 moieties.

### Example 5: Bispecific, Trivalent eIg-Fab Molecules Targeting HER3 and CD3

For the generation of bispecific, trivalent eIg-Fab molecules, i.e. fusion of a Fab fragment to an eIg molecule, different combinations of i) two Fab domains derived from antibody 3-43 (anti-HER3; Schmitt et al., 2017, mAbs, 9:831-843), i.e. Fv domains specific for HER3 fused to the C_{H}1/C_{L} heterodimer of IgG1, ii) one Fab domain specific for CD3 fused to modified C_{H}2 domain of IgE (hetEHD2), and iii) a heterodimeric Fc part (knob-into-hole technology) were used (FIG. 4). The CD3 binding site was derived from a humanized version of the anti-CD3 mAb UCHT1. The bispecific, trivalent eIg-Fab molecules consist of four different polypeptide chains with different arrangement of the HER3 and CD3 binding sites (see FIG. 4 for details). Thus, the bispecific, trivalent eIg-Fab molecules exhibit two antigen-binding sites for HER3 and one binding site for CD3. The molecular structure of the bispecific, bivalent eIg molecule (eIgl) with one binding site for HER3 and one for CD3 is described in example 2. The sequences of the light and heavy chain are described in Table 2.

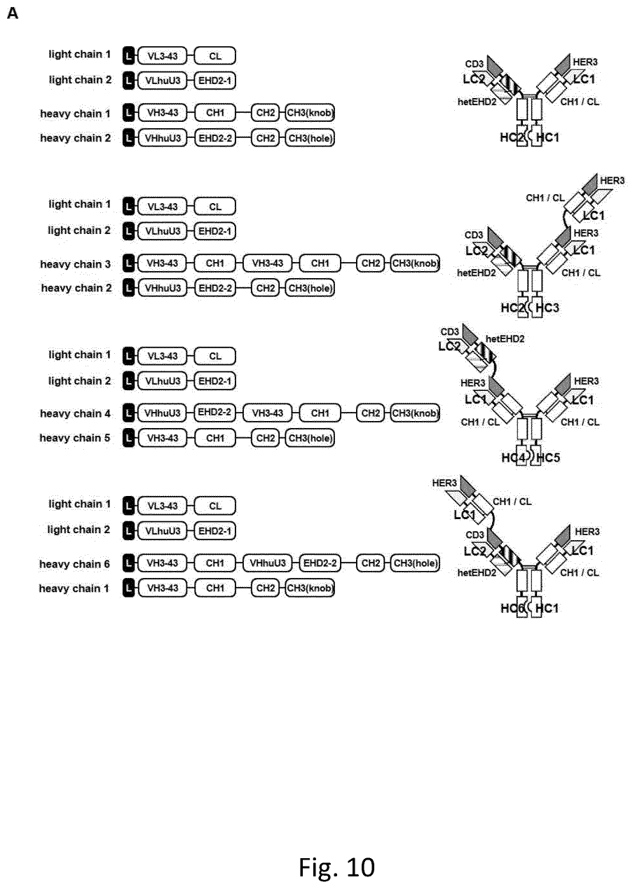

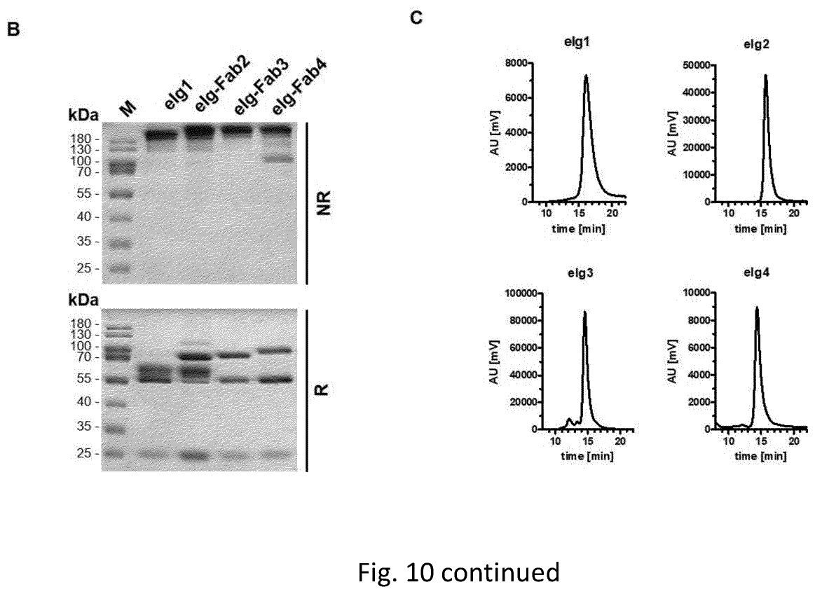

TABLE 2
Different polypeptide chains for the generation
of eIg molecules. The different bispecific eIg and
eIg-Fab molecules were generated by using the two
light chains and two of the heavy chains. FIG. 10
shows the usage of the different molecules.

| Chain of the eIg molecules | SEQ ID NO |
|---|---|
| Light chain 1-LC1 | 13 |
| Light chain 2-LC2 | 15 |
| Heavy chain 1-HC1 | 14 |
| Heavy chain 2-HC2 | 16 |
| Heavy chain 3-HC3 | 20 |
| Heavy chain 4-HC4 | 39 |
| Heavy chain 5-HC5 | 40 |
| Heavy chain 6-HC6 | 41 |

The bispecific, bi- or trivalent eIg molecules were produced in transiently transfected HEK293-6E cells using polyethylenimine (PEI; linear, 25 kDa, Sigma-Aldrich, 764604) as transfection reagent. Supernatants were harvested 96 hours post transfection and proteins were purified by protein A affinity chromatography. Protein purity was confirmed in SDS-PAGE analysis (FIG. 10B), where all bispecific, trivalent eIg-Fab molecules revealed three major bands under reducing conditions: one band at approximately 77 kDa corresponding to the heavy chains V_{H}3-43-C_{H}1-linker-V_{H}huU3-EHD2-Fcₕₒₗₑ, V_{H}huU3-EHD2-linker-V_{H}3-43-C_{H}1-Fc_{knob} or V_{H}3-43-C_{H}1-linker-V_{H}3-43-C_{H}1-Fc_{knob}, respectively, a second band at approximately 55 kDa representing the V_{H}3-43-C_{H}1-Fc_{knob}, V_{H}3-43-C_{H}1-Fcₕₒₗₑ or the V_{L}huU3-EHD2-1(N39Q), and a third band at approximately 26 kDa corresponding to the two different light chains (V_{L}3-43-C_{L}λ and V_{L}huU3-EHD2-1(N39Q)). For the bivalent, bispecific eIg molecule two bands at approximately 55 kDa and 26 kDa, corresponding to the heavy chains V_{H}3-43-C_{H}1⁻Fc_{knob} and V_{H}huU3-EHD2-Fcₕₒₗₑ and the light chains V_{L}huU3-EHD2-1(N39Q) and V_{L}3-43-C_{L}λ, respectively, were observed. One major band above 180 kDa was observed for all eIg-Fab molecules under non-reducing conditions, corresponding to the intact antibodies. Purity, integrity and homogeneity of the bispecific, bi- and trivalent eIg molecules was determined by size-exclusion chromatography using a Waters 2695 HPLC and a TSKgel SuperSW mAb HR column (Tosoh Bioscience) at a flow rate of 0.5 ml/min with 0.1 M Na₂HPO₄/NaH₂PO₄, 0.1 M Na₂SO₄, pH 6.7 as mobile phase. Here, all proteins eluted as one major peak corresponding to the correctly assembled molecules (FIG. 10C).

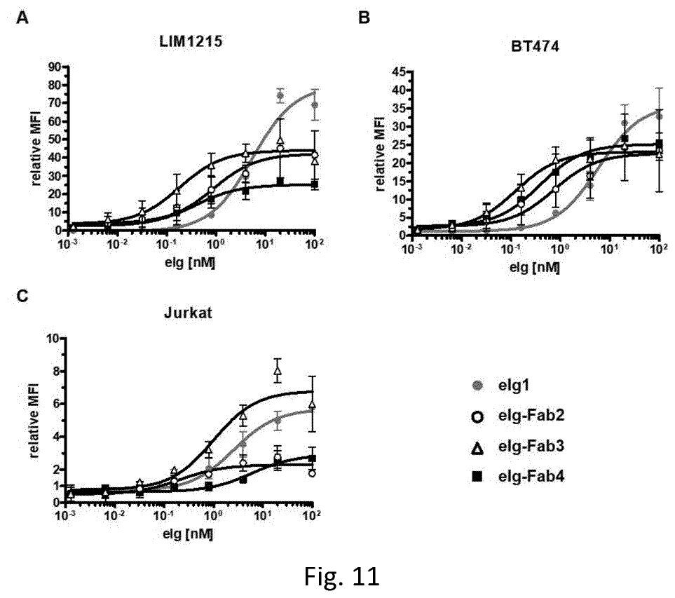

Binding of the eIg-Fab molecules to cancer cell lines exhibiting different HER3 levels (LIM1215: 19,877 HER3/cell; BT474: 11,244 HER/cell) and a CD3-expressing cell line (Jurkat) was determined by flow cytometry (FIG. 11). Adherent cells were washed with PBS and shortly trypsinized at 37° C. Trypsin was quenched with FCS-containing medium and removed by centrifugation (500×g, 5 min.). 1×10⁵ target cells were incubated with serial dilutions of eIg or eIg-Fab molecules diluted in PBA (PBS containing 2% (v/v) FCS, 0.02% (w/v) NaN₃) for one hour at 4° C. Bound proteins were detected using a PE-conjugated anti-human Fc antibody (Jackson ImmunoResearch Laboratories Inc.). Fluorescence was measured by MACSQuant® Analyzer 10 (Miltenyi Biotec) and data were analyzed using FlowJo (Tree Star). Relative mean fluorescence intensities (MFI) were calculated as followed: relative MFI=((MFIₛₐₘₚₗₑ−(MFI_{detection}−-MFI_{cells}))/MFI_{cells}). On the two HER3-expressing cancer cell lines, the bispecific, trivalent eIg-Fab molecules showed superior binding compared to the bispecific, bivalent eIg1. EC₅₀ values for the bispecific, trivalent eIg-Fab molecules were in the low nanomolar range, whereas the bispecific, bivalent eIg1 molecule showed up to factor 50 weaker binding (FIG. 11A, B) (Table 3). On the CD3-expressing human cell line Jurkat, eIg1 (SEQ ID NOs: 13, 14, 15, 16) and eIg-Fab4 (SEQ ID NOs: 13, 14, 15, 41) showed similar binding with EC₅₀ values of 5.1±2.2 nM and 5.9±0.5 nM, respectively, while eIg-Fab2 (SEQ ID NOs: 13, 15, 16, 20) and eIg-Fab3 (SEQ ID NO: 13, 15, 39, 40) showed a stronger binding with EC₅₀ values of 0.2±0.08 nM and 1.1±0.5 nM, respectively (Table 3). Thus, the higher valency for HER3 of the trivalent bispecific eIgs led to a superior binding capacity on HER3-expressing cancer cells compared to the bivalent bispecific eIg. In contrast, different binding to CD3-expressing Jurkat cells of the trivalent bispecific eIg molecules indicates an influence of the position of the CD3 binding site within the eIg-Fab molecules on CD3 binding.

TABLE 3
Cell binding of eIg molecules analyzed by flow cytometry.
EC₅₀ values (nM) were determined using HER3-expressing cells
(LIM1215 and BT474) and the CD3-expressing Jurkat cells in
combination with a serial dilution of the eIg molecules.
Mean ± SD, n = 3.

| | HER3 | EC₅₀ [nM] | | | |
|---|---|---|---|---|---|
| cell line | expression | eIgl | eIg-Fab2 | eIg-Fab3 | eIg-Fab4 |
| LIM1215 | 19,877 | 4.0 ± 2.2 | 1.3 ± 0.9 | 0.4 ± 0.3 | 0.21 ± 0.2 |
| | HER3/cell | | | | |
| BT474 | 11,244 | 4.8 ± 2.9 | 1.7 ± 0.7 | 0.2 ± 0.4 | 0.6 ± 0.5 |
| | HER3/cell | | | | |
| Jurkat | — | 5.1 ± 2.2 | 0.2 ± 0.08 | 1.1 ± 0.5 | 5.9 ± 0.5 |

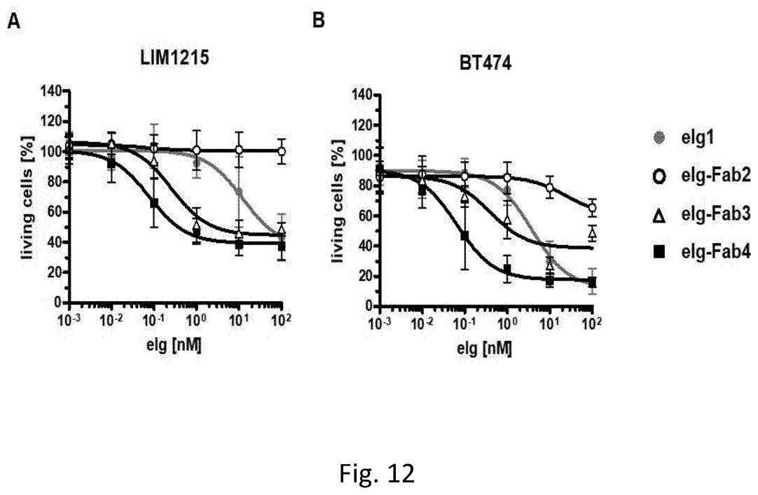

Cytotoxic effects of PBMCs on target cells mediated by bispecific, bi- and trivalent eIg molecules were determined using HER3-positive cell lines with different antigen expression (LIM1215: 19,877 HER3/cell; BT474: 11,244 HER/cell). Target cells (2×10⁴ cells/well) were incubated with bispecific, bi- or trivalent eIg or eIg-Fab antibodies for 15 min at RT prior to addition of PBMCs (E:T ratio of 10:1). After incubation for 3 days at 37° C., supernatants were discarded and viable target cells were stained using crystal violet. After washing and drying, remaining dye was solved in methanol (50 μl/well) and optical density was measured at 550 nM using the Tecan spark (Tecan). The eIg molecules were able to redirect unstimulated PBMCs to lyse HER3-expressing cancer cells in a concentration-dependent manner (FIG. 12). The cytotoxic activity of the eIg antibodies was evaluated by potency (EC₅₀ value in cell killing) (Table 4).

On the two cell lines LIM1215 and BT474, highest potency was observed for the bispecific, trivalent eIg-Fab4 with EC₅₀ values of 0.1±0.09 nM and 0.2±0.1 nM, respectively. An approximately 4-fold weaker potency was observed for the bispecific, trivalent molecule eIg-Fab3. Further reduced potencies were observed for the bispecific, bivalent eIg molecule eIg1 with EC₅₀ values of 5.8±2.9 nM (LIM1215) and 3.8±0.6 nM (BT474). Very low cell killing potency was seen for eIg-Fab2. In summary, these experiments show that the trivalent bispecific eIg-Fab can mediated increased cytotoxicity through T-cell retargeting compared to the bivalent bispecific eIg, i.e. due to the additional target-binding site. However, the experiments also show that potency is affected by the molecular composition, i.e. position of the HER3 and CD3 binding sites within the eIg-Fab molecules.

TABLE 4
Cytotoxic activity of eIg and eIg-Fab molecule on different tumor
cell lines. EC₅₀ values (nM) were determined using HER3-expressing
tumor cells and a serial dilution of eIg and eIg-Fab molecules.
Mean ± SD, n = 3, n.d. = not determined.

| | | EC₅₀ [nM] | | | |
|---|---|---|---|---|---|
| cell line | HER3 expression | eIg1 | eIg-Fab2 | eIg-Fab3 | eIg-Fab4 |
| LIM1215 | 19,877 HER3/cell | 5.8 ± 2.9 | n.d. | 0.4 ± 0.3 | 0.1 ± 0.1 |
| BT-474 | 11,244 HER3/cell | 3.8 ± 0.6 | n.d. | 0.5 ± 0.5 | 0.2 ± 0.1 |

### Example 6: Bivalent and Bispecific eIg Molecule Targeting HER3 and FAP

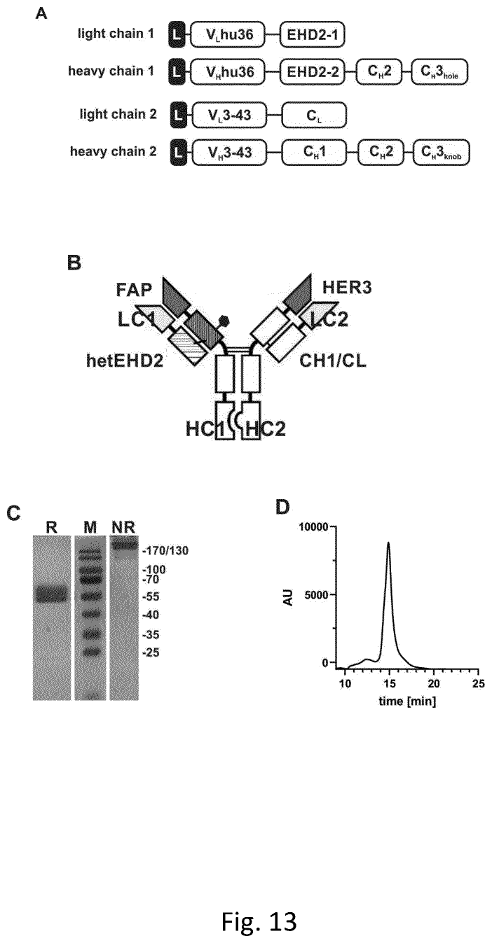

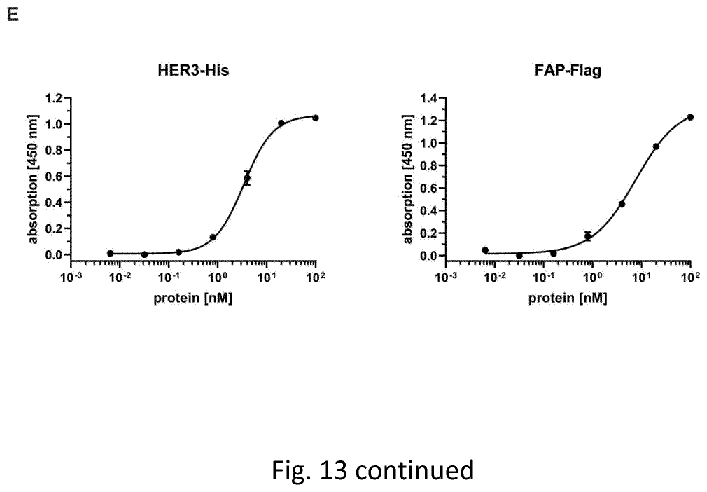

Based on the different variable domains from therapeutic antibodies, a bispecific and bivalent eIg molecule was generated by combining i) an Fv domain specific for HER3 (antibody 3-43; Schmitt et al., 2017, mAbs 9, 831-843), with the C_{H}1/C_{L} heterodimer of IgG1, ii) an Fv domain specific for fibroblast activation protein (FAP; hu36; Fabre et al., 2020, Clin. Cancer Res. 26, 3420-3430) with the modified C_{H}2 domain of IgE (hetEHD2), fused to iii) a heterodimeric Fc part (knob-into-hole technology). Thus, the bispecific molecule consists of four different polypeptide chains: the light chain V_{L}3-43-C_{L}λ (SEQ ID NO: 13) pairing with the heavy chain V_{H}3-43-C_{H}1-Fc_{knob} (SEQ ID NO: 14), and the light chain V_{L}hu36-EHD2-1(N39Q) (SEQ ID NO: 43) pairing with the heavy chain V_{H}hu36-EHD2-2-Fcₕₒₗₑ (SEQ ID NO: 42). The bispecific, bivalent eIg molecule exhibit one antigen binding site for HER3 and one binding site for FAP. The respective polypeptide chains are exemplarily shown in FIG. 13A, the resulting antibody molecule is schematically shown in FIG. 13B.

The bispecific, bivalent eIg molecule was expressed in transiently transfected HEK293-6E cells after co-administration of the four plasmids encoding for both heavy chains and both light chains, using polyethylenimine as transfection reagent. Protein secreted into cell culture supernatant was purified using Protein A affinity chromatography. SDS-PAGE analysis revealed 4 major bands under reducing conditions: two bands at approximately 55 kDa corresponding to the two heavy chains, and two bands at approximately 23 kDa corresponding to the two light chains of the antibody. Under non-reducing conditions, one major band at approximately 200 kDa was observed corresponding to the intact antibody composed of the four different polypeptide chains (FIG. 13C). Purity, integrity and homogeneity of the eIg molecule was confirmed by size-exclusion chromatography (FIG. 13D). Binding of the eIg molecule to the extracellular domain (ECD) of HER3 (aa 21-643) and FAP (aa 32-728) was determined by ELISA. The His-tagged HER3 protein and the Flag-tagged FAP protein were coated onto polystyrene microtiter plates at a concentration of 2 μg/ml diluted in PBS. Remaining binding sites were blocked with PBS, 2% skimmed milk (MPBS). Plates were then incubated with serial dilution of the bispecific eIg antibody. After washing, bound antibodies were detected with an HRP-conjugated anti-human Fc antibody using HER3-His or FAP-Flag as immobilized antigen. TMB and H₂O₂ were used as substrate. The eIg antibody showed concentration-dependent binding to HER3-His and FAP-Flag with EC₅₀ values in the nanomolar range (3.4 nM for HER3; 7.6 nM for FAP) (FIG. 13E). These experiments confirmed binding of the eIg antibody to both antigens, HER3 and FAP.

### Example 7: Bivalent and Monospecific eIg Molecule Targeting HER3

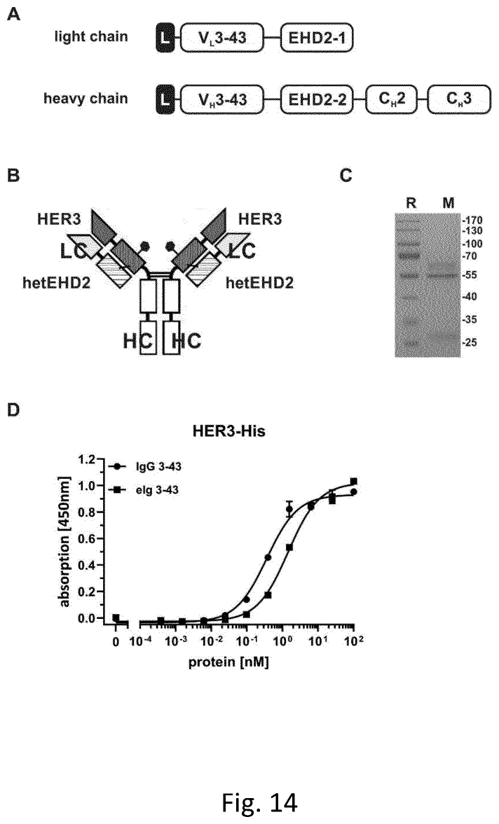

An anti-HER3 bivalent eIg molecule was generated by combining variable domains of an anti-HER3 antibody (3-43) (Schmitt et al., 2017, mAbs 9, 831-843) with the modified C_{H}2 domains of IgE (hetEHD2) fused to further homodimerizing Fc regions. Thus, this molecule consists of two different polypeptide chains: the light chain V_{L}3-43-EHD2-1 (SEQ ID NO: 23) and the heavy chain V_{H}3-43-EHD2-2-Fc (SEQ ID NO: 22). The monospecific, bivalent eIg molecule exhibits two identical antigen-binding sites for HER3. The respective polypeptide chains are exemplarily shown in FIG. 14A, the resulting antibody molecule is schematically shown in FIG. 14B.

The monospecific, bivalent eIg molecule was expressed in transiently transfected HEK293-6E cells after co-administration of the two plasmids encoding for the heavy chain and the light chain, using polyethylenimine as transfection reagent. Protein secreted into cell culture supernatant was purified using Protein A affinity chromatography. SDS-PAGE analysis revealed 2 major bands under reducing conditions: one band at approximately 55 kDa corresponding to the heavy chain, and one band at approximately 25 kDa corresponding to the light chain of the antibody (FIG. 14C). Binding of the eIg molecule to the extracellular domain (ECD) of HER3 (aa 21-643) was determined by ELISA. The His-tagged HER3 protein was coated onto polystyrene microtiter plates at a concentration of 2 μg/ml diluted in PBS. Remaining binding sites were blocked with PBS, 2% skimmed milk (MPBS). Plates were then incubated with serial dilution of the monospecific eIg antibody. After washing, bound antibodies were detected with an HRP-conjugated anti-human Fc antibody. TMB and H₂O₂ were used as substrate. The monospecific, bivalent eIg antibody showed concentration-dependent binding to HER3-His with EC₅₀ values in the nanomolar range (1.4 nM for HER3) (FIG. 14D). The parental antibody IgG 3-43 showed similar binding to immobilized HER3. This experiment confirmed binding of the monospecific, bivalent eIgs antibody to HER3 as immobilized antigen. In summary, monospecific and bivalent eIg molecules with two antigen-binding sites against the same epitope were successfully generated.

### Example 8: Tetravalent and Bispecific eIg Molecule Targeting HER3 and EGFR

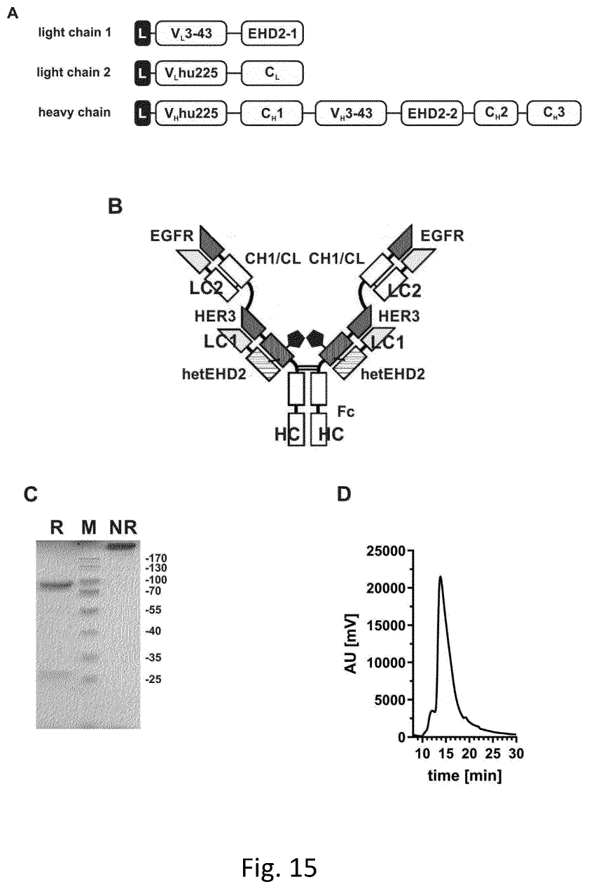

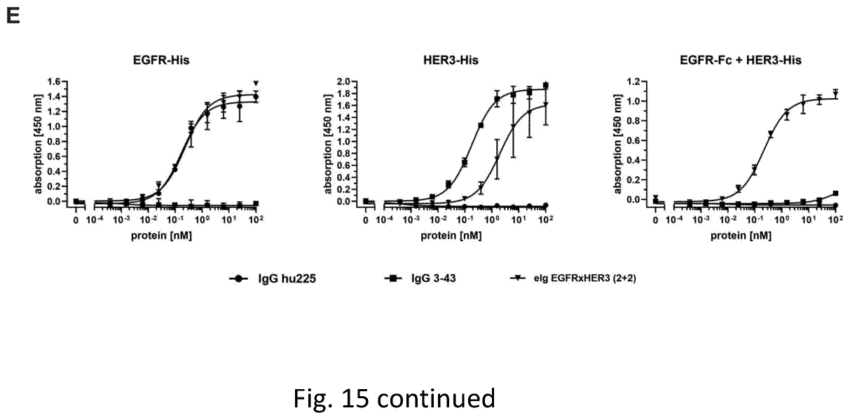

A bispecific tetravalent eIg-Fab molecule was generated by combining the variable domains of an anti-EGFR antibody (hu225; Seifert et al., 2014, Mol Cancer Ther 13, 101-111), with the C_{H}1/C_{L} heterodimer of IgG1 and the variable domains of an anti-HER3 (3-43; Schmitt et al., 2017, mAbs 9, 831-843) with the modified C_{H}2 domains of IgE (hetEHD2) combined further with a homodimerizing Fc part. Thus, the tetravalent, bispecific molecules consist of three different polypeptide chains: the light chains V_{L}3-43-EHD2-1 (SEQ ID NO: 23) and V_{L}hu225-C_{L}κ (SEQ ID NO: 25), and the heavy chain V_{H}hu225-C_{H}1-V_{H}3-43-EHD2-2-Fc (SEQ ID NO: 24). The two Fab moieties in the heavy chain were separated by a linker containing 10 amino acids (GGSGG)₂. The bispecific, tetravalent eIg molecule exhibits two antigen binding sites for EGFR and two binding sites for HER3. The respective polypeptide chains are exemplarily shown in FIG. 15A, the resulting binding molecule is schematically shown in FIG. 15B.

The bispecific, tetravalent eIg-Fab molecule was expressed in transiently transfected HEK293-6E cells after co-administration of the three plasmids encoding for the heavy chain and both light chains, using polyethylenimine as transfection reagent. Protein secreted into cell culture supernatant was purified using Protein A affinity chromatography. SDS-PAGE analysis revealed 3 major bands under reducing conditions: one band at approximately 80 kDa corresponding to the heavy chains, and two bands at approximately 26 kDa corresponding to the two light chains of the antibody (FIG. 15C). Under non-reducing conditions, one major band at approximately 250 kDa was observed corresponding to the intact antibody composed of the three different polypeptide chains. Purity, integrity and homogeneity of the bispecific, tetravalent eIg-Fab molecule was confirmed by size-exclusion chromatography (FIG. 15D).

Binding of this tetravalent eIg-Fab molecule to the extracellular domains of EGFR (aa 20-643) and HER3 (aa 21-643) was determined by ELISA (FIG. 15E). The His-tagged EGFR or HER3 proteins were immobilized to polystyrene microtiter plates at a concentration of 2 μg/ml diluted in PBS. Remaining binding sites were blocked with PBS, 2% skimmed milk (MPBS). Plates were then incubated with serial dilutions of the bispecific eIg-Fab antibody. After washing, bound antibodies were detected with an HRP-conjugated anti-human Fc antibody.TMB and H₂O₂ were used as substrate. The bispecific, tetravalent antibody showed a concentration-dependent binding to EGFR-His and HER3-His with EC₅₀ values in the nanomolar range (0.39±0.25 nM for EGFR; 3.3±2.7 nM for HER3) (FIG. 15E) (Table 5). Simultaneous binding of both antigens was demonstrated with immobilized EGFR-Fc incubated with a serial dilution of the bispecific eIg-Fab molecule and subsequently with the HER3-His protein. Bound HER3-His was detected via HRP-conjugated anti-His detection antibody. A concentration-dependent binding was observed with EC₅₀ values of approximately 0.23±0.01 nM. In summary, the data demonstrates the generation of bispecific and tetravalent eIg molecules with 2+2 binding sites using the eIg platform technology.

TABLE 5
Binding of antibodies to EGFR and HER3 analyzed by ELISA.
EC₅₀ values (nM) were determined using EGFR-His, HER3-His,
or EGFR-Fc and HER3-His as antigens and the serial dilution
of the antibodies. Mean ± SD, n = 3, n.d. = not determined.

| | | | EGFR-Fc + |
|---|---|---|---|
| | EGFR-His | HER3-His | HER3-His |
| IgG hu225 | 0.17 ± 0.005 nM | n.d. | n.d. |
| IgG 3-43 | n.d. | 0.18 ± 0.05 nM | n.d. |
| eIg EGFR × HER3 | 0.39 ± 0.25 nM | 3.3 ± 2.7 nM | 0.23 ± 0.01 nM |

### Example 9: Bivalent and Bispecific Fab-eFab Molecule Targeting HER3 and EGFR

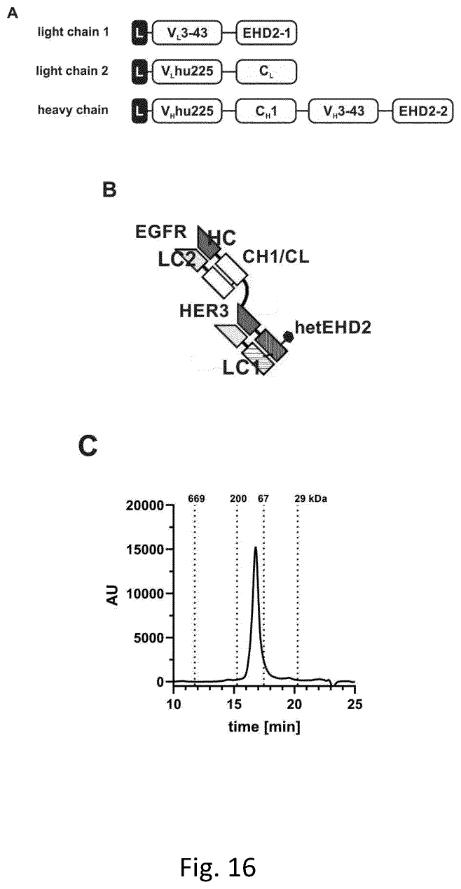

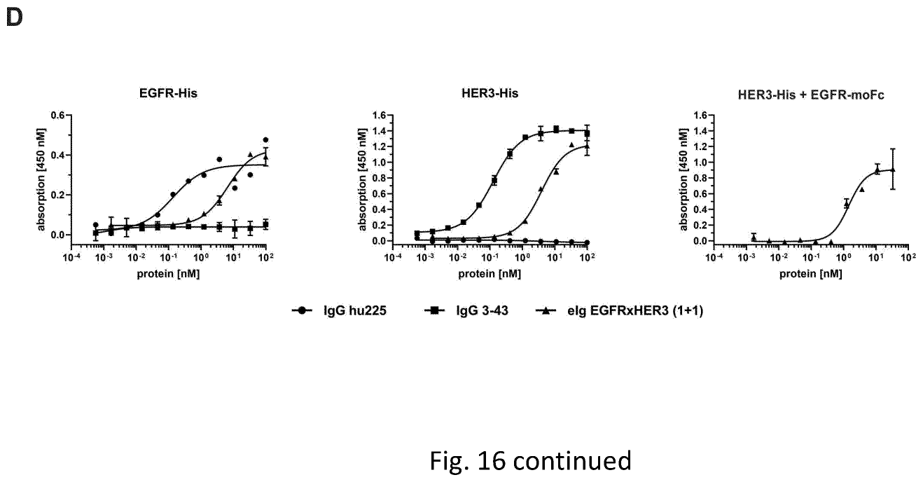

A bispecific and bivalent Fab-eFab molecule, i.e. lacking an Fc region, was generated by combining the variable domains of an anti-EGFR (hu225; Seifert et al., 2014, Mol Cancer Ther 13, 101-111) with the C_{H}1/C_{L} heterodimer of IgG1, the variable domains of an anti-HER3 (3-43; Schmitt et al., 2017, mAbs 9, 831-843) with the modified C_{H}2 domain of IgE (hetEHD2). The Fab-eFab molecule consisted of the light chains V_{L}3-43-EHD2-1 (SEQ ID NO: 23) and V_{L}hu225-C_{L}κ (SEQ ID NO: 25), and the “heavy chain” V_{H}hu225-C_{H}1-V_{H}3-43-EHD2-2 (SEQ ID NO: 44). The bispecific, bivalent Fab-eFab molecule exhibited one antigen-binding site for EGFR and one binding site for HER3. The heavy chain contained a His-tag on the C-terminus for purification and detection of the molecule. The two Fab/eFab moieties were connected via a linker containing 10 amino acids (GGSGG)₂. The respective polypeptide chains are exemplarily shown in FIG. 16A, the resulting antibody molecule is schematically shown in FIG. 16B. Purity, integrity and homogeneity of the Fab-eFab molecule was confirmed by size-exclusion chromatography (FIG. 16C).

Binding of this Fab-eFab molecule to the extracellular domains of EGFR (aa 20-643) and HER3 (aa 21-643) was analyzed by ELISA. The His-tagged EGFR and HER3 proteins were coated onto polystyrene microtiter plates at a concentration of 2 μg/ml diluted in PBS. Remaining binding sites were blocked with PBS, 2% skimmed milk (MPBS). Plates were then incubated with serial dilution of the bispecific Fab-eFab antibody or the parental antibodies (IgG hu225, IgG 3-43). After washing, bound antibodies were detected with an HRP-conjugated anti-human Fab antibody using both immobilized antigens. TMB and H₂O₂ were used as substrate. The Fab-eFab antibody showed concentration-dependent binding to EGFR-His and HER3-His with EC₅₀ values in the nanomolar range (6.5 nM for EGFR; 3.9 nM for HER3) (FIG. 16D) (Table 6). The parental antibodies showed a higher binding capacity to the immobilized antigens, due to two antigen-binding sites in each molecule. Simultaneous binding to both antigens was demonstrated using immobilized HER3-His incubated with a serial dilution of the Fab-eFab molecule followed by the EGFR-moFc protein as second soluble antigen. Bound second antigen was detected via HRP-conjugated anti-mouse Fc detection antibody. A concentration-dependent binding was observed with EC₅₀ values of approximately 1.4 nM. These experiments confirmed binding of the bispecific, bivalent Fab-eFab antibody to EGFR and HER3.

TABLE 6
Binding of antibodies to EGFR and HER3 analyzed by ELISA.
EC₅₀ values (nM) were determined using EGFR-His, HER3-His,
or HER3-His and EGFR-moFc as antigens in combination with
the serial dilution of the antibodies. Mean ± SD, n = 1,
n.d. = not determined, — = not performed.

| | | | HER3-His + |
|---|---|---|---|
| | EGFR-His | HER3-His | EGFR-moFc |
| IgG hu225 | 0.14 nM | n.d. | — |
| IgG 3-43 | n.d. | 0.12 nM | — |
| eIg EGFR × HER3 | 6.5 nM | 3.9 nM | 1.4 nM |

### Example 10: Bivalent and Bispecific eIg Molecule Targeting FAP and Mouse CD3—Different Composition of the Variable Domains Binding Mouse CD3

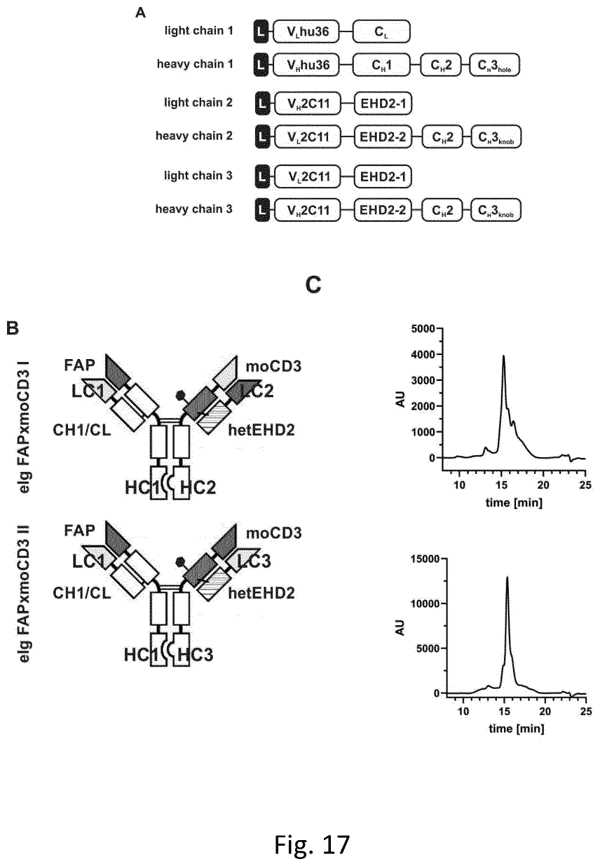

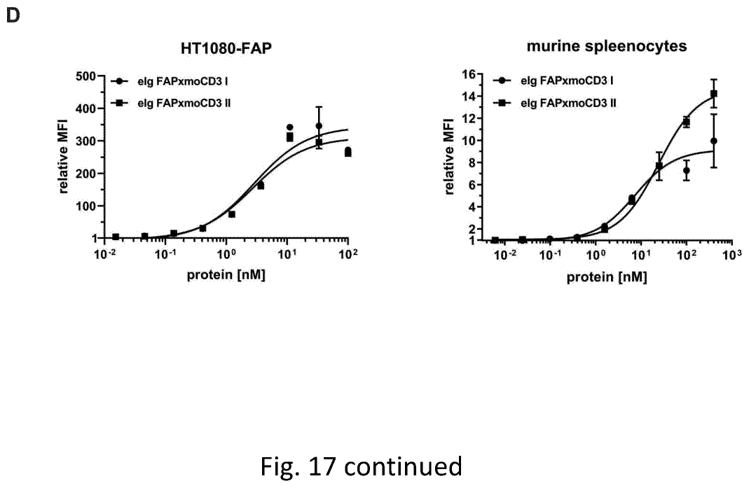

Bispecific bivalent eIg molecules were generated by combining the variable domains of an anti-fibroblast activation protein (FAP) antibody (hu36; Fabre et al., 2020, Clin Cancer Res. 26, 3420-3430) with the C_{H}1/C_{L} heterodimer of IgG1, the variable domains of an anti-mouse CD3 (2C11; Leo et al., 1987, Proc Natl Acad USA 84, 1374-1378) with the modified C_{H}2 domain of IgE (hetEHD2), further fused to a heterodimerizing Fc part (knob-into-hole technology). The compositions of the polypeptide chains are shown in FIG. 17A. Thus, the first part of the eIg molecules consisted of the FAP-targeting arm with the light chain V_{L}hu36-CLκ (SEQ ID NO: 45) and the heavy chain V_{H}hu36-C_{H}1-Fc(hole) (SEQ ID NO: 46). Molecule I consisted additionally of the mouse CD3-targeting arm with the light chain V_{H}2C11-EHD2-1 (SEQ ID NO: 47) and the heavy chain V_{L}2C11-EHD2-2-Fc(knob) (SEQ ID NO: 48). The second molecule consisted of the light chain V_{L}2C11-EHD2-1 (SEQ ID NO: 49) and the heavy chain V_{H}2C11-EHD2-2-Fc(knob) (SEQ ID NO: 50). The resulting antibody molecules are schematically shown in FIG. 17B. Purity, integrity and homogeneity of both eIg molecules was confirmed by size-exclusion chromatography (FIG. 17C).

Binding of both bispecific eIg antibodies to FAP-expressing HT1080-FAP cells and mouse spleenocytes (expressing mouse CD3) was analyzed by flow cytometry. Adherent HT1080-FAP cells were washed with PBS and shortly trypsinized at 37° C. Trypsin was quenched with FCS containing medium and removed by centrifugation (500 xg, 5 minutes). For harvesting murine immune cells from the spleen, a spleen was extracted and a homogeneous cell suspension was prepared by mashing the speen through a cell strainer. After collection of the cells by centrifugation (250 xg, 7 minutes), erythrocytes were removed and spleenocytes were resuspended in medium. 100,000 HT1080-FAP cells or 200,000 murine spleenocytes were incubated with serial dilutions of the bispecific eIg antibodies diluted in PBA (PBS containing 2% (v/v) FCS, 0.02% (w/v) NaN3) for one hour at 4° C. Cells were washed twice using PBA. Bound antibodies were detected using PE-labeled anti-human Fc secondary antibody, which was incubated for another hour at 4° C. After washing, median fluorescence intensity (MFI) was measured with a Milltenyi MACSQuant® Analyzer 10. Relative MFIs (to unstained cells) were calculated by Flow Jo (Tree star) and Excel. The eIg molecules bound to the cells in a concentration-dependent manner. The eIg molecules I and II bound with EC₅₀ values of 2.9 nM and 2.7 nM to HT1080-FAP cells and 7.3 nM and 24.2 nM to murine spleenocytes, respectively (FIG. 17D) (Table 7).

TABLE 7
Cell binding of antibodies analyzed by flow cytometry.
EC₅₀ values (nM) were determined using FAP-expressing
HT1080-FAP or murine CD3-expressing murine spleenocytes
as target cells in combination with the serial dilution of the
antibodies. Mean ± SD, n = 1.

| | | Murine |
|---|---|---|
| | HT1080-FAP | spleenocytes |
| eIg FAP × moCD3 I | 2.9 nM | 7.3 nM |
| eIg FAP × moCD3 II | 2.7 nM | 24.2 nM |

### Example 11: Lack of Binding of eIg molecules to Fcc-receptor I

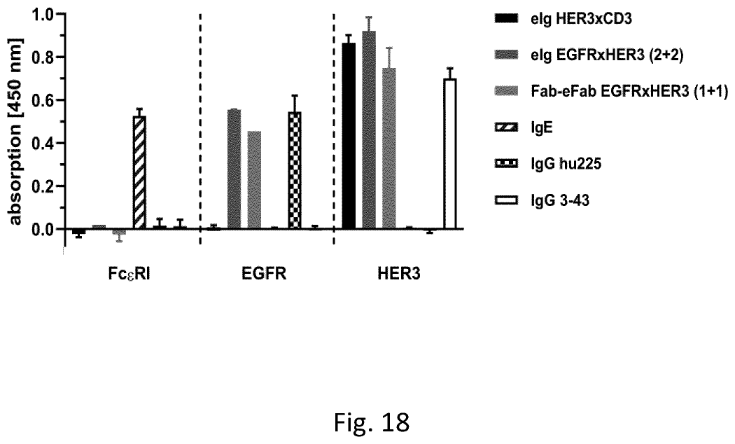

In the eIg technology, a modified C_{H}2 domain derived from human IgE is used as heterodimerization module. It was investigated if hetEHD2 comprising antibody molecules lack binding to the high-affinity IgE receptor FcεRI. FcεRI was generated as an Fc fusion protein comprising the extracellular region of FcεRI. In ELISA experiments (FIG. 18), the purified FcεRI was used as immobilized antigen incubated with different eIg derivatives including an eIg specific for HER3 and CD3 (example 5), a tetravalent, bispecific eIg-Fab specific for EGFR and HER3 (example 10) and a bivalent, bispecific Fab-eFab specific for EGFR and HER3 (example 9). As control, EGFR and HER3 were used as additional immobilized antigens to confirm binding of the antibodies to these receptors. FcεRT, EGFR and HER3 were coated onto polystyrene microtiter plates at a concentration of 2 μg/ml diluted in PBS. Remaining binding sites were blocked with PBS, 2% skimmed milk (MPBS). Plates were then incubated with 50 nM of eIg, or eIg-Fab, and Fab-eFab molecules to study binding to the different immobilized receptors. Additionally, human IgE was included in this experiment to confirm binding to the FCC receptors. After washing, bound antibodies were detected with an anti-human Fab antibody. TMB and H₂O₂ were used as substrate. IgE specifically bound to FcεRT. The three eIg derivatives did not show any binding to FcεRT, but strongly recognized their respective target antigens, i.e. eIg HER3×CD3 bound to HER3, and eIg-Fab EGFRxHER3 and Fab-eFab EGFRxHER3 bound to EGFR and HER3. Control antibody IgG 3-43, from which the HER3-binding site is derived, bound to HER3 and IgG hu225, from which the EGFR binding site is derived bound to EGFR. No binding to FcεRT was seen with these two control IgG antibodies (FIG. 18). Thus, this experiment demonstrates that eIg molecules lack binding to the IgE receptor FcεRT, which is expressed by mast cells and basophiles. We can therefore exclude that EHD2 or hetEHD2 comprising molecules lead to activation of these immune cells by binding to FccRI.

### Example 12: Bispecific eIg Molecules for Targeting of the SARS-CoV-2 Spike Protein

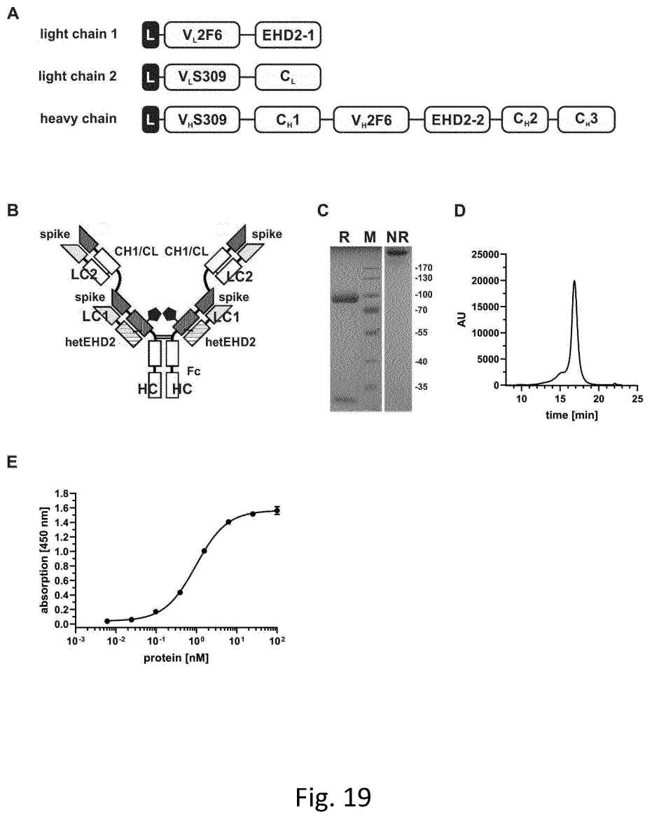

Bispecific eIg-Fab molecules were generated for dual targeting of different epitopes of the spike protein of the SARS-CoV-2 virus. For this approach, published antibodies were used that bind to the RBD domain of the spike protein of the virus: P2B-2F6 (Ju et al., 2020, Nature 584, 115-119) and S309 (Pinto et al., 2020, Nature 583, 290-295). The variable domains of S309 were combined with the CH1/C_{L} heterodimer of IgG1, and the variable domains of P2B-2F6 were combined with hetEHD2 domains, further combined with a homodimerizing Fc part to generate a symmetric tetravalent, bispecific eIg-Fab molecule. The antibody consists of the light chains V_{L}S309-CLκ (SEQ ID NO: 51) and V_{L}P2B-2F6-EHD2-1 (SEQ ID NO: 52), and of the heavy chain V_{H}S309-C_{H}1-V_{H}P2B-2F6-EHD2-2-Fc (SEQ ID NO: 53). The respective polypeptide chains are exemplarily shown in FIG. 19A, the resulting antibody molecule is schematically shown in FIG. 19B.

The bispecific, tetravalent eIg-Fab molecule was expressed in transiently transfected HEK293-6E cells after co-administration of the three plasmids encoding for the heavy chain and both light chains, using polyethylenimine as transfection reagent. Protein secreted into cell culture supernatant was purified using Protein A affinity chromatography. SDS-PAGE analysis revealed 3 major bands under reducing conditions: one band at approximately 85 kDa corresponding to the heavy chains, and two bands at approximately 26 kDa corresponding to the two light chains of the antibody (FIG. 19C). Under non-reducing conditions, one major band at approximately 250 kDa was observed corresponding to the intact antibody composed of the three different polypeptide chains. Purity, integrity and homogeneity of the bispecific, tetravalent eIg-Fab molecule was confirmed by size-exclusion chromatography (FIG. 19D).

Binding of this eIg-Fab molecule to the RBD domain of the spike protein was determined by ELISA. The His-tagged RBD was coated onto polystyrene microtiter plates at a concentration of 2 μg/ml diluted in PBS. Remaining binding sites were blocked with PBS, 2% skimmed milk (MPBS). Plates were then incubated with serial dilution of the bispecific eIg-Fab antibody. After washing, bound antibodies were detected with an HRP-conjugated anti-human Fc antibody. TMB and H₂O₂ were used as substrate. The bispecific, tetravalent eIg-Fab antibody showed a concentration-dependent binding to RBD-His antigen with EC₅₀ values in the nanomolar range (1.0 nM) (FIG. 19E). These experiments confirm binding of the bispecific, tetravalent eIg-Fab antibody to RBD.

1. A binding molecule comprising:
a first polypeptide chain comprising a first binding domain (BD1) and a first modified EHD2 domain (EHD2-1), and
a second polypeptide chain comprising a second binding domain (BD2) and a second modified EHD2 domain (EHD2-2),
wherein the amino acid sequences of EHD2-1 and EHD2-2 are different from each other and each is selected from (i) an amino acid sequence with at least 70% amino acid identity to SEQ ID NO: 1 and which does not have a Cys at position 14, or (ii) an amino acid sequence with at least 70% amino acid identity to SEQ ID NO: 1 and which does not have a Cys at position 102,
wherein BD1 and BD2 together form an antigen binding site, and wherein EHD2-1 and EHD2-2 are covalently bound to each other.

2. The binding molecule according to claim 1, wherein BD1 and BD2 are different from each other and each is selected from a V_{H} and a V_{L} or from a variable region of a TCR α-chain and a variable region of a TCR β-chain.

3. The binding molecule according to any one of claims 1, further comprising a first Fc chain.

4. The binding molecule according to claim 1, further comprising a third binding domain (BD3) and a fourth binding domain (BD4), wherein BD3 and BD4 together form an antigen binding site, preferably wherein BD3 and BD4 are different from each other and each is selected from a V_{H} and a V_{L} or from a variable region of a TCR α-chain and a variable region of a TCR β-chain.

5. The binding molecule according to claim 4, wherein the binding molecule further comprises a third polypeptide chain, preferably wherein the binding molecule further comprises a third and a fourth polypeptide chain, more preferably further comprising a second Fc chain.

6. The binding molecule according to claim 5, wherein the third polypeptide chain comprising BD3 further comprises one of
(i) a C_{H}1 domain,
(ii) a C_{L} domain,
(iii) a first modified EHD2 domain (EHD2-1), and
(iv) a second modified EHD2 domain (EHD2-2),
and wherein the fourth polypeptide chain comprising BD4 further comprises
in case of (i) a C_{L} domain,
in case of (ii) a C_{H}1 domain,
in case of (iii) a second modified EHD2 domain (EHD2-2), and
in case of (iv) a first modified EHD2 domain (EHD2-1),
wherein the amino acid sequences of EHD2-1 and EHD2-2 are different from each other and each is selected from an amino acid sequence with at least 70% amino acid identity to SEQ ID NO: 1 and which does not have a Cys at position 14, or an amino acid sequence with at least 70% amino acid identity to SEQ ID NO: 1 and which does not have a Cys at position 102.

7. The binding molecule according to claim 4, further comprising a fifth binding domain (BD5) and a sixth binding domain (BD6), wherein BD5 and BD6 together form an antigen binding site, preferably wherein BD5 and BD6 are different from each other and each is selected from a V_{H} and a V_{L} or from a variable region of a TCR α-chain and a variable region of a TCR β-chain, more preferably wherein the binding molecule further comprises
(i) a C_{H}1 domain,
(ii) a C_{L} domain,
(iii) a first modified EHD2 domain (EHD2-1), or
(iv) a second modified EHD2 domain (EHD2-2)
connected to BD5, and
in case of (i) a C_{L} domain,
in case of (ii) a C_{H}1 domain,
in case of (iii) a second modified EHD2 domain (EHD2-2), and
in case of (iv) a first modified EHD2 domain (EHD2-1) connected to BD6, wherein the amino acid sequences of EHD2-1 and EHD2-2 are different from each other and each is selected from an amino acid sequence with at least 70% amino acid identity to SEQ ID NO: 1 and which does not have a Cys at position 14, or an amino acid sequence with at least 70% amino acid identity to SEQ ID NO: 1 and which does not have a Cys at position 102.

8. The binding molecule according to claim 6, further comprising a seventh binding domain (BD7) and an eighth binding domain (BD8), wherein BD7 and BD8 together form an antigen binding site, preferably wherein BD7 and BD8 are different from each other and each is selected from a V_{H} and a V_{L} or from a variable region of a TCR α-chain and a variable region of a TCR β-chain.

9. The binding molecule according to claim 8, further comprising
(i) a C_{H}1 domain,
(ii) a C_{L} domain,
(iii) a first modified EHD2 domain (EHD2-1), or
(iv) a second modified EHD2 domain (EHD2-2)
connected to BD7, and
in case of (i) a C_{L} domain,
in case of (ii) a C_{H}1 domain,
in case of (iii) a second modified EHD2 domain (EHD2-2), and
in case of (iv) a first modified EHD2 domain (EHD2-1) connected to BD8, wherein the amino acid sequences of EHD2-1 and EHD2-2 are different from each other and each is selected from an amino acid sequence with at least 70% amino acid identity to SEQ ID NO: 1 and which does not have a Cys at position 14, or an amino acid sequence with at least 70% amino acid identity to SEQ ID NO: 1 and which does not have a Cys at position 102.

10. The binding molecule according to claim 1, wherein none, one or more of the modified EHD2 domains carries one N-glycan.

11. The binding molecule according to claim 1,
wherein the Cys at position 14 of SEQ ID NO: 1 is substituted by an amino acid selected from the group consisting of Ser, Gly, Ala, Thr, Gln, Asn, and Tyr, preferably Ser, and/or
wherein the Cys at position 102 of SEQ ID NO: 1 is substituted by an amino acid selected from the group consisting of Ser, Gly, Ala, Thr, Gln, Asn, and Tyr, preferably Ser.

12. The binding molecule according to any of the preceding claims claim 1, further comprising a single amino acid substitution at position N39 in one or more of the modified EHD2 domains, preferably wherein the single amino acid substitution is N39Q.

13. A nucleic acid or set of nucleic acids encoding the binding molecule according to claim 1.

14. A vector comprising the nucleic acid or set of nucleic acids of claim 13.

15. A host cell comprising the vector according to claim 14.

16. A pharmaceutical composition comprising the binding molecule according to claim 1, and a pharmaceutically acceptable carrier.

17. A method of treating cancer, comprising administering to a patient in need thereof a therapeutically effective amount to treat the cancer of the binding molecule according to claim 1.


SEQUENCE LISTING (53 sequences)

SEQ ID NO: 1 - protein, 106 aa, Homo sapiens
DFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSN

SEQ ID NO: 2 - protein, 106 aa, Artificial Sequence
  modified EHD2 C14X
  misc_feature (14)..(14): Xaa can be any naturally occurring amino acid
DFTPPTVKILQSSXDGGGHFPPTIQLLCLVSGYTPGTINITWLEDGQVMDVDLSTASTTQEGELASTQSELTLSQKHWLSDRTYTCQVTYQGHTFEDSTKKCADSN

SEQ ID NO: 3 - protein, 106 aa, Artificial Sequence
  modified EHD2 C102X
  misc_feature (102)..(102): Xaa can be any naturally occurring amino acid
DFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKXADSN

SEQ ID NO: 4 - protein, 106 aa, Artificial Sequence
  modified EHD2 C14S
DFTPPTVKILQSSSDGGGHFPPTIQLLCLVSGYTPGTINITWLEDGQVMDVDLSTASTTQEGELASTQSELTLSQKHWLSDRTYTCQVTYQGHTFEDSTKKCADSN

SEQ ID NO: 5 - protein, 106 aa, Artificial Sequence
  modified EHD2 C14S N39Q
DFTPPTVKILQSSSDGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 6 - protein, 106 aa, Artificial Sequence
  modified EHD2 C102S
DFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSN

SEQ ID NO: 7 - protein, 106 aa, Artificial Sequence
  modified EHD2 N39Q C102S
DFTPPTVKILQSSCDGGGHFPPTIQLLCLVSGYTPGTIQITWLEDGQVMDVDLSTASTTQEGELASTQSELTLSQKHWLSDRTYTCQVTYQGHTFEDSTKKSADSN

SEQ ID NO: 8 - protein, 20 aa, Artificial Sequence
  Igkappa leader
METDTLLLWVLLLWVPGSTG

SEQ ID NO: 9 - protein, 221 aa, Artificial Sequence
  VL5D5-EHD2-1 (N39Q)
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTDFTPPTVKILQSSSDGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 10 - protein, 454 aa, Artificial Sequence
  VH5D5-EHD2-2-Fchole
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 11 - protein, 221 aa, Artificial Sequence
  VL5D5-EHD2-2
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSN

SEQ ID NO: 12 - protein, 454 aa, Artificial Sequence
  VH5D5-EHD2-1(N39Q)-Fchole
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSDFTPPTVKILQSSSDGGGHFPPTIQLLCLVSGYTPGTIQITWLEDGQVMDVDLSTASTTQEGELASTQSELTLSQKHWLSDRTYTCQVTYQGHTFEDSTKKCADSNGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 13 - protein, 214 aa, Artificial Sequence
  VL3-43-CLkappa
QAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 14 - protein, 455 aa, Artificial Sequence
  VH3-43-CH1-Fcknob
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 215 aa, Artificial Sequence
  VLhuU3-EHD2-1(N39Q)
DIQMTQSPSSLSASVGDRVTITCRASQDIRNYLNWYQQKPGKAPKLLIYYTSRLHSGVPSRFSGSGSGTDFTFTISSLQPEDIATYYCQQGNTLPWTFGQGTKLEIKRTDFTPPTVKILQSSSDGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 16 - protein, 457 aa, Artificial Sequence
  VHhuU3-EHD2-2-Fchole
QVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 17 - protein, 463 aa, Artificial Sequence
  VH5D5-EHD2-2-Linker-VH3-43-CH1
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGTGGSGGGGSGGQVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 463 aa, Artificial Sequence
  VH3-43-CH1-Linker-VH5D5-EHD2-2
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGTGGSGGGGSGGEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSN

SEQ ID NO: 19 - protein, 692 aa, Artificial Sequence
  VH3-43-CH1-Linker-VH5D5-EHD2-2-Fchole
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGTGGSGGGGSGGEVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 20 - protein, 693 aa, Artificial Sequence
  VH3-43-CH1-Linker-VH3-43-CH1-Fcknob
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGTGGSGGGGSGGQVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 690 aa, Artificial Sequence
  VH5D5-EHD2-2-Linker-VH3-43-CH1-Fchole
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGTGGSGGGGSGGQVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 458 aa, Artificial Sequence
  VH3-43-EHD2-2-Fc
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 216 aa, Artificial Sequence
  VL3-43-EHD2-1
QAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLGTDFTPPTVKILQSSSDGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 24 - protein, 690 aa, Artificial Sequence
  VHhu225-CH1-Linker-VH3-43-EHD2-2-Fc
EVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGSGGGGSGGQVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 214 aa, Artificial Sequence
  VLhu225-CLk
DIQLTQSPSFLSASVGDRVTITCRASQSIGTNIHWYQQKPGKAPKLLIKYASESISGVPSRFSGSGSGTEFTLTISSLQPEDFATYYCQQNNNWPTTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 690 aa, Artificial Sequence
  VH3-43-EHD2-2-Linker-VHhu225-CH1-Fc
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGTGGSGGGGSGGEVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 238 aa, Artificial Sequence
  VH3-43-EHD2-2(C102A)-His
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKAADSNAAAHHHHHH

SEQ ID NO: 28 - protein, 238 aa, Artificial Sequence
  VH3-43-EHD2-2(C102W)-His
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKWADSNAAAHHHHHH

SEQ ID NO: 29 - protein, 238 aa, Artificial Sequence
  VH3-43-EHD2-2(C102N)-His
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKNADSNAAAHHHHHH

SEQ ID NO: 30 - protein, 238 aa, Artificial Sequence
  VH3-43-EHD2-2(C102T)-His
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKTADSNAAAHHHHHH

SEQ ID NO: 31 - protein, 238 aa, Artificial Sequence
  VH3-43-EHD2-2(C102S)-His
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNAAAHHHHHH

SEQ ID NO: 32 - protein, 214 aa, Artificial Sequence
  VL3-43-EHD2-1(C14A, N39Q)
QAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLDFTPPTVKILQSSADGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 33 - protein, 214 aa, Artificial Sequence
  VL3-43-EHD2-1(C14T, N39Q)
QAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLDFTPPTVKILQSSTDGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 34 - protein, 214 aa, Artificial Sequence
  VL3-43-EHD2-1(C14N, N39Q)
QAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLDFTPPTVKILQSSNDGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 35 - protein, 214 aa, Artificial Sequence
  VL3-43-EHD2-1(C14W, N39Q)
QAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLDFTPPTVKILQSSWDGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 36 - protein, 214 aa, Artificial Sequence
  VL3-43-EHD2-1(C14S, N39Q)
QAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLDFTPPTVKILQSSSDGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 37 - protein, 214 aa, Artificial Sequence
  VL3-43-EHD2-1(C14S)
QAGLTQPPAVSVAPGQTASITCGRDNIGSRSVHWYQQKPGQAPVLVVYDDSDRPAGIPERFSGSNYENTATLTISRVEAGDEADYYCQVWGITSDHVVFGGGTKLTVLDFTPPTVKILQSSSDGGGHFPPTIQLLCLVSGYTPGTINITWLEDGQVMDVDLSTASTTQEGELASTQSELTLSQKHWLSDRTYTCQVTYQGHTFEDSTKKCADSN

SEQ ID NO: 38 - protein, 238 aa, Artificial Sequence
  VH3-43-EHD2-2(N39Q, C102S)-His
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSDFTPPTVKILQSSCDGGGHFPPTIQLLCLVSGYTPGTIQITWLEDGQVMDVDLSTASTTQEGELASTQSELTLSQKHWLSDRTYTCQVTYQGHTFEDSTKKSADSNAAAHHHHHH

SEQ ID NO: 39 - protein, 692 aa, Artificial Sequence
  VHhuU3-EHD2-2-Linker-VH3-43-CH1-Fcknob
QVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGGSGGGGSGGQVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGTDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 454 aa, Artificial Sequence
  VH3-43-CH1-Fchole
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGTDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 41 - protein, 692 aa, Artificial Sequence
  VH3-43- CH1-Linker-VHhuU3-EHD2-2- Fchole
QVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGSGGGGSGGQVQLVQSGAEVKKPGSSVKVSCKASGGTFSGYTMNWVRQAPGQGLEWMGLINPYKGVSTYNGKFKDRVTITADKSTSTAYMELSSLRSEDTAVYYCARSGYYGDSDWYFDVWGQGTLVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGTDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 42 - protein, 455 aa, Artificial Sequence
  VHhu36-EHD2-2-Fchole
QVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 218 aa, Artificial Sequence
  VLhu36-EHD2-1
DIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKRDFTPPTVKILQSSSDGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 44 - protein, 470 aa, Artificial Sequence
  VHhu225-CH1-linker-VH3-43-EHD2-2-His
EVQLVESGGGLVQPGGSLRLSCAASGFSLTNYGVHWVRQAPGKGLEWLGVIWSGGNTDYNTPFTSRFTISRDNSKNTLYLQMNSLRAEDTAVYYCARALTYYDYEFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGSGGGGSGGQVQLQQSGPGLVKPSQTLSLTCAISGDSVSSNRAAWNWIRQSPSRGLEWLGRTYYRSKWYNDYAQSLKSRITINPDTPKNQFSLQLNSVTPEDTAVYYCARDGQLGLDALDIWGQGTMVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNAAAHHHHHH

SEQ ID NO: 45 - protein, 219 aa, Artificial Sequence
  VLhu36-CLk
DIQMTQSPSSLSASVGDRVTITCRASKSVSTSAYSYMHWYQQKPGKAPKLLIYLASNLESGVPSRFSGSGSGTDFTLTISSLQPEDFATYYCQHSRELPYTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 451 aa, Artificial Sequence
  VHhu36-CH1-Fchole
QVQLVQSGAEVKKPGASVKVSCKASGYTFTENIIHWVRQAPGQGLEWMGWFHPGSGSIKYNEKFKDRVTMTADTSTSTVYMELSSLRSEDTAVYYCARHGGTGRGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGTDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 47 - protein, 222 aa, Artificial Sequence
  VH2C11-EHD2-1
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSDFTPPTVKILQSSSDGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 48 - protein, 441 aa, Artificial Sequence
  VL2C11-EHD2-2-Fcknob
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGTDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 213 aa, Artificial Sequence
  VL2C11-EHD2-1
DIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKDFTPPTVKILQSSSDGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 50 - protein, 450 aa, Artificial Sequence
  VH2C11-EHD2-2-Fcknob
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNGTDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 214 aa, Artificial Sequence
  VLS309-CLk
EIVLTQSPGTLSLSPGERATLSCRASQTVSSTSLAWYQQKPGQAPRLLIYGASSRATGIPDRFSGSGSGTDFTLTISRLEPEDFAVYYCQQHDTSLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 216 aa, Artificial Sequence
  VLP2B-2F6-EHD2-1
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSNNLVCGGGTKLTVLDFTPPTVKILQSSSDGGGHFPPTIQLLCLVSGYTPGTIQITWLEDGQVMDVDLSTASTTQEGELASTQSELTLSQKHWLSDRTYTCQVTYQGHTFEDSTKKCADSN

SEQ ID NO: 53 - protein, 699 aa, Artificial Sequence
  VHS309-CH1-VHP2B-2F6-EHD2-2-Fc
QVQLVQSGAEVKKPGASVKVSCKASGYPFTSYGISWVRQAPGQGLEWMGWISTYNGNTNYAQKFQGRVTMTTDTSTTTGYMELRRLRSDDTAVYYCARDYTRGAWFGESLIGGFDNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGSGGGGSGGQVQLQESGPGLVKPSETLSLTCTVSGYSISSGYYWGWIRQPPGKGLEWIGSIYHSGSTYYNPSLKTRVTISVDTSKNQFSLKLSSVTAADTAVYYCARAVVGIVVVPAAGRRAFDIWGQGTMVTVSSDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKSADSNDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK